(19) United States
(12) Patent Application Publication
(10) Pub. No.: US 2007/0286806 A1
(43) Pub. Date: Dec. 13, 2007

(54) Methods and Compositions for Modulating a Steroid Receptor

(75) Inventors: Stephen Lye, Totonto (CA); Xuesen Dong, Aurora (CA)

Correspondence Address: Mary E. Bak;Howson and Howson, 501 Office Center Drive, Suite 210, Fort Washington, PA, 19034, US

(73) Assignee: Mount Sinai Hospital, Toronto, ON (CA)

(21) Appl. No.: 10/586,125

(22) PCT Filed: Jan. 14, 2005
(86) PCT No.: PCT/CA05/00042

§ 371 Date: Jun. 28, 2007

Related U.S. Application Data
(60) Provisional application No. 60/536,598, filed on Jan. 15, 2004.

(51) Int. Cl. A61K 31/7088; A61K 38/00; A61P 43/00; C12Q 1/68; G01N 33/50; G01N 33/53

(52) U.S. Cl. 424/9.2; 435/6; 435/7.1; 436/501; 436/86; 436/94; 514/2; 514/44

(57) ABSTRACT

Methods, compositions, and uses are provided for modulating a steroid receptor or process mediated by a steroid receptor in a cell by administering a polypyridimine tract binding protein-associated splicing factor (PSF) polypeptide, a polynucleotide encoding the polypeptide, an isolated complex of a PSF polypeptide and a steroid receptor, and/or an agonist or antagonist thereof, in an effective amount to modulate the steroid receptor or process. Particular aspects of the invention relate to detection, monitoring, modulation, treatment and/or prevention of the onset of labor.

### FIELD OF THE INVENTION

The invention relates to methods and compositions for modulating a steroid receptor, in particular a progesterone receptor. In aspects the invention provides methods and compositions for diagnosing, inducing, and preventing the onset of labor.

### BACKGROUND OF THE INVENTION

Progesterone receptor (PR) modulators have been widely used in regulation of female reproduction systems and in treatment of female hormone dependent diseases. Progesterone receptor (PR) is a member of the steroid receptor superfamily of ligand dependent transcriptional factors. In the human myometrium, PR is transcribed as full-length PR-B and an N-terminally (164 amino acid) truncated PR-A isoform (5). PR-A is generally a weaker transcriptional activator than PR-B (6, 7 and 8) and can also act as a repressor of PR-B as well as of other steroid receptors (9 and 10). Upon ligand binding through the hormone binding domain (HBD), the activated PR undergoes a conformation change enabling it to bind to specific progesterone response elements (PREs) through its DNA binding domain (DBD). This in turn facilitates recruitment of the general transcription machinery, either directly (11) or indirectly via cofactors (12 and 13), which act to positively or negatively modulate the transcription rate of target genes. Two common transcriptional activation domains exist within PRs, a hormone-dependent activation function domain (AF2) in the C-terminal HBD, and a ligand-independent domain (AFI) in the N-terminal region (14). In addition, PRB possesses a third activation domain (AF3) within its unique N-terminal region (15). Interactions between the N-terminus AFs (AF1/AF3) and the AF2 domain (either direct or indirect via co-regulators) elicit maximal hormone dependent activity (16). Nuclear receptor co-regulators have a multifaceted role in regulating gene transcription. Besides the autonomous activation domains in steroid receptor co-activators (SRCs) and repression domains in NcoR and SMRT, many co-regulators possess acetylase (such as SRCs, CBP/p300, pCAF), or deacetylase (such as HDAC-1/-2) activities (17). Moreover, in the case of E6-AP and RPF-1, nuclear receptor activation by these co-regulators can be enhanced through their ubiquitin ligase activity, which is separable from their co-activation functions (18 and 19).

Progesterone is an essential regulator of the reproductive events associated with the establishment and maintenance of pregnancy through its ligand-activated progesterone receptor (PR) (1). Progesterone actions include the suppression of genes encoding contraction-associated proteins (CAPs, e.g. oxytocin receptor, prostaglandin receptor, connexin43) that are required for myometrial activation and the onset of labor. In the human, progesterone levels remain elevated through labor raising a paradox as to how labor can be initiated. Even in species where progesterone levels fall at term, concentrations are likely sufficiently high to inhibit CAP gene expression. This suggests there must be an active mechanism for inducing a functional withdrawal of progesterone at term. A blockade of progesterone receptor signaling in the myometrium could induce a “functional withdrawal” of progesterone that would result in the initiation of labor (2). A number of mechanisms have been proposed to effect such a functional withdrawal including, changes in the expression of PR or of PR isoforms (3) as well as altered transcriptional activity of PR as a result of changes in the expression of essential coregulators (both co-activators and co-repressors) (4).

One of most significant problems in obstetrics is the management of pre-term labor. A significant number of pregnancies progressing past 20 weeks of gestation experience premature labor and delivery, which is a leading cause of infant deaths and long-term neurological handicaps, including cerebral palsy, blindness, deafness, and developmental defects. To date the efforts to reduce the incidence of pre-term labor have been unsuccessful. This is attributed to a number of factors including the difficulties in identifying pregnancies at risk for pre-term labor, the lack of reliable diagnosis of pre-term labor, and the inability to effectively intervene.

### SUMMARY OF THE INVENTION

The invention provides methods, compositions, and uses of polypyridimine tract binding protein-associated splicing factor (PSF) polypeptides, PSF polynucleotides, or complexes of a PSF polypeptide and a steroid receptor including without limitation a PR polypeptide, a glucocorticoid receptor (GR), or an androgen receptor (AR), or agonists or antagonists thereof. A PSF polypeptide, PSF polynucleotide, PR polypeptide, and a complex of a PSF polypeptide and a steroid receptor, used in the invention are referred to, and further defined herein as “PSF Polypeptide”, “PSF Polynucleotide”, “PR Polypeptide”, and “PSF Complex”, respectively.

The invention relates to a method of modulating a steroid receptor such as a PR Polypeptide, GR, or AR, in a cell by administering a PSF polypeptide, a PSF polynucleotide, a PSF Complex, and/or an agonist or antagonist thereof, in an amount that modulates the steroid receptor, in particular modulates, binding of a steroid receptor to a hormone response element in the cell. In an aspect, the invention provides a method of modulating a PR polypeptide in a cell by administering a PSF polypeptide, a PSF polynucleotide, or a PSF-PR Complex, or an agonist or antagonist thereof, in an amount that modulates the PR Polypeptide, in particular modulates binding of a PR polypeptide to a progesterone response element (PRE) in the cell.

The invention also relates to a method of modulating a process mediated by a steroid receptor, in particular a PR Polypeptide, GR, or AR, in a cell comprising administering to the cell an amount of a PSF Polypeptide, a PSF Polynucleotide, a PSF Complex, and/or an agonist or antagonist thereof, effective to modulate the process. In an aspect the steroid receptor is a PR Polypeptide and the process involves suppression of the genes required for myometrial activation and the onset of labor.

The invention also provides a method of modulating a process mediated by a steroid receptor, in particular a PR Polypeptide, GR, or AR, in a cell comprising administering to a patient having a condition mediated by a steroid receptor an effective amount of a PSP Polypeptide, a PSF Polynucleotide, a PSF complex, and/or an agonist or antagonist thereof. In an aspect, a method of modulating a process mediated by a PR Polypeptide in a cell is provided comprising administering to a patient having a condition mediated by a PR Polypeptide an effective amount of a PSF Polypeptide, a PSF Polynucleotide, PSF-PR Complex, or an agonist or antagonist thereof.

The invention provides a method to co-repress steroid receptor transactivation, in particular progesterone receptor transactivation, GR transactivation, or AR transactivation, in a cell comprising administering to the cell a PSF Polypeptide, a PSF Polynucleotide, a PSF Complex, and/or an agonist thereof. In an aspect, a method is provided to co-repress progesterone receptor transactivation in a cell comprising administering to the cell a PSF Polypeptide, a PSF Polynucleotide, a PSF-PR Complex, and/or an agonist thereof.

The invention provides a method of inhibiting transactivation domains of a steroid receptor, in particular, a PR Polypeptide, in a cell comprising administering to the cell a PSF Polypeptide, a PSF Polynucleotide, a PSF Complex, and/or an agonist thereof.

The invention provides a method to stimulate or enhance steroid receptor transactivation, in particular progesterone receptor transactivation, GR transactivation, or AR transactivation, in a cell comprising administering to the cell an antagonist of a PSF Polypeptide, a PSF Polynucleotide, and/or a PSF Complex. In an aspect, a method is provided to stimulate or enhance progesterone receptor transactivation in a cell comprising administering to the cell an antagonist of a PSF Polypeptide, a PSF Polynucleotide, and/or a PSF-PR Complex.

The invention provides a method of stimulating transactivation domains of a steroid receptor, in particular, a PR Polypeptide, in a cell comprising administering to the cell an antagonist of a PSF Polypeptide, a PSF Polynucleotide, and/or a PSF Complex.

The invention further relates to a method of repressing steroid receptor signaling in a cell comprising administering an amount of a PSF Polypeptide, a PSF Polynucleotide, PSF Complex, and/or an agonist or antagonist thereof, effective to inhibit the binding of a DNA binding domain of an activated steroid receptor to a hormone response element. In an aspect, the steroid receptor is progesterone receptor and the hormone response element is PRE.

The invention further relates to a method of stimulating steroid receptor signaling in a cell comprising administering an amount of an antagonist of a PSF Polypeptide, a PSF Polynucleotide, and/or a PSF Complex, effective to stimulate or increase binding of a DNA binding domain of an activated steroid receptor to a hormone response element. In an aspect, the steroid receptor is progesterone and the hormone response element is PRE.

In an aspect the invention relates to a method of modulating (e.g. inhibiting or blocking) a steroid receptor signal transduction pathway, in particular a PR Polypeptide, GR, or AR, comprising administering an effective amount of a PSF Polypeptide, a PSF Polynucleotide, a PSF Complex, and/or an agonist or antagonist thereof, to modulate the pathway. In an aspect the invention provides a method of modulating (e.g. inhibiting or blocking) a progesterone receptor signal transduction pathway in a cell involving a PSF Polypeptide and a PR Polypeptide comprising administering an effective amount of a PSF Polypeptide, a PSF Polynucleotide, PSF-PR Complex, and/or an agonist or antagonist thereof, to modulate the pathway.

The invention still further relates to a method of modulating (e.g. enhance or increase) degradation of a steroid receptor, in particular a PR Polypeptide, GR, or AR, in a cell comprising administering to the cell an amount of a PSF Polypeptide, a PSF Polynucleotide, a PSF complex, and/or an agonist or antagonist thereof that modulates degradation of a steroid receptor. In an aspect, a method of modulating degradation of a PR Polypeptide in a cell is provided comprising administering to the cell an amount of a PSF Polypeptide, a PSF Polynucleotide, PSF-PR Complex, and/or an agonist or antagonist thereof, that modulates degradation of a PR Polypeptide in the cell.

The invention also provides a method of blocking or interfering with steroid receptor binding with DNA in a cell comprising administering an amount of a PSF Polypeptide, a PSF Polynucleotide, a PSF complex, and/or an agonist thereof, to block or interfere with the binding. In an aspect, a method of blocking, decreasing, or interfering with binding of a PR Polypeptide with DNA in a cell is provided comprising administering an amount of a PSF Polypeptide, a PSF Polynucleotide, a PSF-PR complex, and/or an agonist thereof, to block or interfere with the binding.

The invention also provides a method of stimulating or increasing steroid receptor binding with DNA in a cell comprising administering an amount of an antagonist of a PSF Polypeptide, a PSF Polynucleotide, and/or a PSF complex to stimulate or increase the binding. In an aspect, a method of stimulating or increasing binding of a PR Polypeptide with DNA in a cell is provided comprising administering an amount of an antagonist of a PSF Polypeptide, a PSF Polynucleotide, and/or a PSF-PR Complex to stimulate or increase the binding.

The invention also provides a method of modulating (e.g. increasing or enhancing) recruitment of HDAC protein complexes in a cell comprising administering an amount of a PSF Polypeptide, a PSF Polynucleotide, a PSF complex (in particular a PSF-PR complex), and/or an agonist or antagonist thereof to modulate recruitment of HDAC protein complexes.

The disruption or promotion of the interaction between molecules in PSF Complexes can be useful in therapeutic procedures. Therefore, the invention features a method for treating a subject or individual having a condition characterized by an abnormality in a steroid receptor signal transduction pathway, in particular a progesterone receptor signal transduction pathway involving an interaction between a PSF Polypeptide and PR Polypeptide, a glucocorticoid receptor signal transduction pathway involving an interaction between a PSF Polypeptide and a GR; and an androgen receptor signal transduction pathway involving an interaction between a PSF Polypeptide and a GR. The condition may also be characterized by an abnormal level of interaction between a PSF Polypeptide and a steroid receptor such as a PR Polypeptide, glucocorticoid receptor, and androgen receptor. The method includes disrupting or promoting the interaction (or signal) in cells in vitro and in vivo. The method also involves inhibiting or promoting the activity of a PSF Complex, in particular a PSF-PR Complex.

The present invention has particular application in modulating labor in a subject. In an aspect, the present invention relates to methods for detecting, treating and/or preventing labor or pre-term labor, or inducing the onset of labor, by modulating a PSF Polypeptide and/or a PSF-PR Complex in a subject.

In an aspect, the invention contemplates a method for regulating the onset of labor in a subject comprising inhibiting or stimulating a PSF Polypeptide, PSF Polynucleotide, PSF-PR Complex, or interaction between a PSF Polypeptide and a PR Polypeptide. In an embodiment of the invention, a method is provided for delaying or inhibiting the onset of labor in a subject comprising administering to the subject an effective amount of an antagonist or inhibitor of a PSF Polypeptide, PSF Polynucleotide, and/or PSF-PR Complex. In an embodiment, a method is provided for controlling pre-term labor sufficiently to extend pregnancy in a subject to as close to full term as possible comprising administering to the subject an effective amount of an antagonist or inhibitor of a PSF Polypeptide, PSF Polynucleotide, and/or PSF-PR Complex.

The invention provides a method of preventing premature labor in a subject susceptible thereto, comprising administration of a labor preventive amount of an antagonist or inhibitor of a PSF Polypeptide, PSF-PR Complex, and/or PSF Polynucleotide to the subject.

In an embodiment of the invention a method is provided for treating a female suffering from, or who may be susceptible to pre-term labor comprising administering therapeutically effective dosages of an antagonist or inhibitor of a PSF Polypeptide, PSF-PR Complex, and/or PSF Polynucleotide. A therapeutically effective dosage is an amount of an antagonist or inhibitor of a PSF Polypeptide, PSF-PR Complex, and/or PSF Polynucleotide effective to maintain progesterone receptor levels or functional activity thus inhibiting the onset of labor.

The invention also provides a method for reducing the risk of pre-term labor in a subject at risk therefore comprising administration of a labor preventive amount of an antagonist or inhibitor of a PSF Polypeptide, PSF-PR Complex, and/or PSF Polynucleotide to the subject.

The invention relates to a method of inhibiting a PR Polypeptide to thereby remove the suppressive action of the PR Polypeptide on the expression of myometrial genes required for labor comprising administering an effective amount of an antagonist or inhibitor of a PSF Polypeptide, PSF Polynucleotide, and/or PSF-PR Complex.

Methods of the invention may also be used to stop or delay labor preparatory to Cesarean delivery. Thus, the invention relates to a method for stopping labor preparatory to Cesarean delivery in a subject in need of such treatment comprising administration of an effective amount of an antagonist or inhibitor of a PSF Polypeptide, PSF-PR Complex, and/or PSF Polynucleotide to the subject.

The present invention is also directed to a method for controlling the timing of parturition in animals, such as domestic animals, so that delivery of the neonates occurs during the daytime and thus can be readily monitored. An antagonist or inhibitor of a PSF Polypeptide, PSF-PR Complex, and/or PSF Polynucleotide is administered to the mother on the evening before the expected delivery to delay parturition so that the delivery occurs during the daylight hours. Delaying the timing of parturition enables proper monitoring of the delivery and neonates, resulting in increased survival rates of the newborn.

The present invention provides a method for initiation of farrowing of pregnant domestic animals within a predictable number of hours. This method involves administration of an antagonist of a PSF Polypeptide, PSF Polynucleotide, and/or PSF-PR Complex to a pregnant animal. An antagonist can be administered in two or more administrations or in a single administration. In an embodiment, an antagonist is administered to pregnant animals in a single administration of an amount effective to induce farrowing at approximately 20 to 25 hours later and to reduce the number of animals born dead.

In another embodiment of the invention, a method is provided for inducing labor in a subject comprising administering an effective amount of a PSF Polypeptide, PSF Polynucleotide, PSF-PR Complex, and/or agonist thereof. In a particular embodiment, a method is provided for inducing labor in a subject comprising administering therapeutically effective dosages of a PSF Polypeptide, PSF Polynucleotide, PSF Complex, and/or an agonist thereof. An amount can be administered which is effective to up-regulate or stimulate a PSF Polypeptide and/or PSF Polynucleotide in the subject.

The invention further provides a method of, and products for, diagnosing, detecting, and monitoring conditions mediated by a PR Polypeptide comprising determining the presence of PSF Polypeptides, PSF Polynucleotides, and/or PSF-PR Complexes.

In an aspect, the invention provides methods for identifying pre-term labor or the onset of labor in a subject comprising detecting a PSF Polypeptide, PSF Polynucleotide, and/or PSF Complex in a sample from the subject. In an embodiment of a diagnostic method of the invention, a method is provided for diagnosing or detecting increased risk of pre-term labor, or onset of labor, in a subject comprising detecting a PSF Polypeptide, PSF Polynucleotide, and/or PSF Complex in a sample from the subject.

The invention also provides methods for identifying agonists and antagonists of a PSF Polypeptide, PSF Complex, or the interaction of a PSF Polypeptide and a PR Polypeptide.

Thus, the invention provides a method of selecting a substance that modulates a steroid receptor, in particular a PR Polypeptide; a PSF Polypeptide; a PSF Complex, in particular a PSF-PR Complex; a process mediated by a steroid receptor, in particular a PR Polypeptide; PSF Polypeptide mediated degradation of a steroid receptor, in particular a PR Polypeptide; a steroid receptor signaling transduction pathway, in particular a progesterone receptor signal transduction pathway; a condition mediated by a steroid receptor, in particular mediated by a PR Polypeptide; steroid receptor transactivation, in particular PR transactivation, and/or inhibits or potentiates the interaction of a steroid receptor and a PSF Polypeptide, comprising assaying for a substance that inhibits or stimulates (i.e. is an agonist or antagonist of) a PSF Polypeptide, a PSF Polynucleotide, or PSF Complex.

In an aspect, the invention relates to a method of selecting a substance that regulates the onset of labor comprising assaying for a substance that inhibits or stimulates (i.e. is an agonist or antagonist of) a PSF Polypeptide and/or a PSF Complex. The substances may be used in the methods of the invention to regulate the onset of labor.

The invention also contemplates the agents, compounds, and substances identified using a method of the invention, in particular agonists and antagonists of a PSF Polypeptide including molecules derived from a PSF Polypeptide, PSF Polynucleotide, a steroid receptor in particular PR Polypeptide, and/or PSF Complex. The agents, compounds, and substances may be used in methods of the invention to modulate a steroid receptor, in particular a PR Polypeptide, a PSF Complex, a process mediated by a steroid receptor, in particular PR Polypeptide, PSF mediated degradation of a steroid receptor in particular progesterone receptor, a steroid receptor signal transduction pathway in particular a progesterone receptor signal transduction pathway, and/or steroid receptor transactivation in particular PR transactivation, and/or inhibit or potentiate the interaction of a PSF Polypeptide and a steroid receptor in particular a PR. The agents, compounds, and substances may be used in the treatment or prevention of a condition mediated by a steroid receptor, in particular a condition mediated by a progesterone receptor.

Particular antagonists of the invention include antibodies specific for a PSF Polypeptide, PSF Complex, chimeric polypeptide or PSF Polynucleotide of the invention. Antibodies may be labeled with a detectable substance and used to detect proteins or complexes of the invention in biological samples, tissues, and cells. Antibodies may have particular uses in therapeutic applications, and in conjugates and immunotoxins as target selective carriers of various agents which have therapeutic effects including chemotherapeutic drugs, toxins, immunological response modifiers, enzymes, and radioisotopes.

PSF Polypeptides, PSF Polynucleotides, PSF Complexes, and agonists and antagonists thereof, and agents, compounds, and substances identified using a method of the invention may be formulated into compositions for administration to subjects. Therefore the present invention also relates to a pharmaceutical composition comprising an effective amount of a PSF Polypeptide, PSF Complex, and/or PSF Polynucleotide, or an agonist or antagonist thereof, or an agent, compound or substance identified using a method of the invention. The pharmaceutical compositions can be used in the methods of the invention. In particular a pharmaceutical composition of the invention can be adapted for administration to a subject for the prevention or treatment of a condition mediated by a steroid receptor, in particular a condition mediated by a progesterone receptor.

In an aspect, the invention relates to a composition adapted for regulating the onset of labor comprising a substance which modulates (e.g. inhibits or stimulates) a PSF Polypeptide, PSF-PR Complex, and/or PSF Polynucleotide, in an amount effective to inhibit or stimulate the onset of labor, and an appropriate carrier, diluent, or excipient.

In an embodiment of the invention, a composition is provided for treating a woman suffering from, or who may be susceptible to pre-term labor, comprising an effective amount of an antagonist or inhibitor of a PSF Polypeptide, PSF-PR Complex, and/or PSF Polynucleotide, and a carrier, diluent, or excipient.

In another embodiment of the invention, a composition is provided for inducing labor in a subject comprising an effective amount of a PSF Polypeptide, PSF-PR Complex, and/or PSF Polynucleotide, or agonist thereof, and a carrier, diluent, or excipient.

The invention further relates to the use of a PSF Polypeptide, PSF Polynucleotide, and/or PSF Complex, or agonist or antagonist thereof, for the manufacture of, or in the preparation of a medicament. The medicament may be used to modulate a steroid receptor, in particular a PR Polypeptide, a PSF Polypeptide, a PSF Complex, a process mediated by a steroid receptor, in particular PR Polypeptide, PSF mediated degradation of a steroid receptor in particular progesterone receptor, a steroid receptor signal transduction pathway in particular a progesterone receptor signal transduction pathway, and/or steroid receptor transactivation in particular PR transactivation, and/or inhibit or potentiate the interaction of a PSF Polypeptide and a steroid receptor in particular a PR. The medicament can be applied to the prevention or treatment of a condition mediated by a progesterone receptor.

In an aspect, the invention relates to the use of an antagonist or inhibitor of a PSF Polypeptide, PSF Polynucleotide and/or PSF-PR Complex, for the manufacture of a medicament useful in modulating the onset of labor.

The invention still further relates to the use of an antagonist or inhibitor of a PSF Polypeptide, PSF Polynucleotide, and/or PSF-PR Complex, for the manufacture of a medicament useful for preventing pre-term or premature labor, reducing the risk of pre-term or premature labor, stopping labor preparatory to Cesarean delivery, or controlling the timing of parturition in animals, such as domestic animals.

The methods, compositions, and uses described herein may utilize substances that are known agonists or antagonists of a PSF Polypeptide, or an agent, compound, or substance identified or assayed by a method described herein.

The invention also relates to kits for carrying out the methods of the invention.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1. Identification of PSF as a PR interacting protein (A) Nuclear and cytoplasmic extracts from SHM cells were incubated with Sepharose beads containing the indicated GST-PR fusion proteins. After washing, the associated proteins were separated on SDS-PAGE and stained with Coomassie blue. Note: WCL represents the whole cell lysate. (B) A protein band running at 100 Da was excised and processed for MALDI mass spectrometry. Four peptide sequences (SEQ ID NOs. 18, 19, 20, and 16) were identified and matched to PSF (position of sequence within PSF is shown). (C) Human PSF domain structures of PSF-A and PSF-F. PSF-A, a 707aa protein with two predicted RNA binding domains (RRM I and RRM II), a coil region (intimately-associated bundles of long alpha-helices), and other regions enriched for proline (P) or proline and glutamine (P, Q). PSF-F (669 amino acids), a shorter spliced version of PSF-A, is identical up to amino acid 662 of PSF-A but diverges with only seven additional amino acids at the C-terminal end (VRMIDVG) (SEQ ID NO. 22).

FIG. 2. PSF interacts with PR in vivo. (A) 293T cells were transient transfected with an expression vector for PRB and/or His-PSF for 18 hours, followed by lysis in NETN buffer with NaCl at 150 mM. Whole cell lysate (WCL) was incubated with PR antibody (C-20, Santa Cruz) or anti-His tag antibody (H-15, Santa Cruz), using protein A/G-sepharose as an absorbent. Resins were washed with NETN containing 100 mM NaCl, and bound proteins were detected by western blotting with antibodies to PR (C-20) or His-PSF (H-15). (B) Western blots of PSF (left) and PR (right) in immunoprecipitates of control mouse IgG or mouse anti-PR (AB52) or anti-PSF (B92) antibody from extracts of T47D cells. (C) SHM cells were maintained in DMEM phenol red free plus 10% stripped FBS and transiently transfected with pM vector fused with or without PSF cDNA, VP16 vector containing PRA or PRB cDNA together with G5-Luc reporter vector and pCH110. Four hours after transfection, culture medium was replaced by fresh medium with or without 10 nM progesterone and maintained for at least 30 hours. Luciferase activity was measured and normalized by β-galatosidase activity. Data shown are the mean of three separate experiments performed in triplicate ±S.E. Note: WCL represents the whole cell lysate.

FIG. 3. Mapping PSF and PR interaction site. (A) GST or GST-PR fusion proteins bound to glutathione-sepharose beads were incubated with ³⁵S-labeled PSF and binding of PSF was assessed by autoradiography. (B) GST or GST-PSF fusion proteins bound to glutathione-sepharose beads were incubated with ³⁵S-labeled PRB and binding of PRB was assessed by autoradiography. Input represents 10% of the material used for each sample in A and B. (C) GST-PSF fusion proteins used in assay B were separated on SDS gel and stained with commassie blue. The protein marker shows the molecular weight.

FIG. 4. PSF inhibit transactivation of PR but not estrogen receptor (ER). (A) SHM cells were transient cotransfected with PRA or PRB expression vectors with or without increasing dose (0.05, 0.1 and 0.2 μg) of His-PSF, together with 3×PRE (upper) or MMTV (lower) luciferase reporter vector. Four hours after transfection, culture medium was replaced with fresh DMEM containing 10 nM progesterone and the incubation continued for at least 30 hours. (B) SHM cells were transiently cotransfected with ERα or ERβ expression vector with (0.2 μg) or without His-PSF, together with 3×ERE luciferase reporter vector. Four hours after transfection, culture medium was replaced with fresh DMEM with (solid bar) or without (empty bar) 10 nM estrodial and incubation continued for at least 30 hours. Luciferase activity was measured and normalized by β-galatosidase activity. Data shown are the mean of three separate experiments performed in triplicate +S.E. Note: the empty vector pcDNA3 was added to the DNA mixture to ensure that the amounts of CMV promoter in all the transfection assays are equal.

FIG. 5. PSF enhanced degradation of PR through the proteosomal pathway. (A) SHM cells were transient transfected (eighteen hours) with PRA or PRB expression vector with or without increasing doses (0.2, 0.5, 1.0 and 2.0 μg) of His-PSF vector as indicated. Whole cell lysate was analyzed by western blot with PR or His tag antibody. NS=non-specific binding. (B) SHM cells were transient transfected (eighteen hours) with PRA or PRB expression vector together with 2.0 μg His tagged PSF₁₋₇₀₇ or truncated PSF₁₋₆₆₂. Culture media was added with or without 60 nM MG132 (Z-Leu-Leu-Leu-a1) as indicated for another 6 hours. Whole cell lysate was analyzed by western blot with PR or His tag antibody. Gβ protein was also detected as the cell lysate loading control.

FIG. 6 A-F. Inhibition of PR activation domains by PSF involves different mechanisms. Segments containing individual activation domains of PR were fused to Gal4 DNA binding domain in pM vector and cotransfected with or without increasing amounts of His-PSF, together with G5 luciferase reporter vector in SHM cells. Progesterone (10 nM) was added to cells transfected with segments containing PR hormone binding domain (HBD). Luciferase activity was measured 30 hours after transfection and normalized to β-galatosidase acitvity. Data shown are the mean of three separate experiments performed in triplicate±S.E. Whole cell lysate from each transfection was western blotted with anti-Gal4 DNA binding domain (DBD) antibody (Santa Cruz) and anti-His tag antibody H-15 (Santa Cruz). Note: the empty vector pSG5 was added to the DNA mixture to ensure that the amounts of SV40 promoter in all the transfection assays are equal.

FIG. 7. PSF contains two inhibitory domains. Various deletion mutants of PSF were linked to pM vector and cotransfected with G5-luc reporter gene. Luciferase activity was measured 30 hours after transfection and normalized to β-galatosidase activity (A). Protein from the cell lysate was used to normalize β-galatosidase activity and also plotted in (B). Data shown are the mean of three separate experiments performed in triplicate±S.E.

FIG. 8. PSF interferes with binding of PR DNA binding domain to PRE. (A) Electrophoretic mobility shift assay was performed with ³²P-labeled PRE incubated with in vitro translated PR_{DBD} (1, 2, 5μl in lane 3 to 5 and 2 μl in lane 6 to 11) and increasing amount of bacterially expressed GST (lane 6 to 8) or GST PSF (lane 9 to 11). TNT lysate (lane 2) was used as negative control. (B) PR-Nuclear extraction (1 and 2 μg) as described in the method was incubated with PRE oligo in a dose dependent manner (lane 2 and 3). Anti-PR antiserum (AB-52) was added to the incubation mixture (lane 4). Addition of increasing dose (1 and 2 μl) of GST or GST-PSF in the same reaction as lane 3 was shown in lane 6, 7 and 9, 10. In the control experiments (lane 5 and 8), GST or GST-PSF was incubated with PRE without adding PR-nuclear extract. All reactions contain 10 nM progesterone.

FIG. 9. Expression profile of PSF and PR. (A) Tissue distribution of PSF in rat was analyzed by Northern blot. Various rat tissues were collected and total RNA was isolated. PSF transcription was detected by a ³²P-labeled PSF probe (described in methods and material). (B) Myometrial tissue was collected during and after pregnancy from rats (n=5 at each time point). Total RNA was extracted and subjected to Northern blotting to assess PSF expression level. The intensity of PSF mRNA bands was quantified by densitometry, and normalized by 18S mRNA. The bars represent mean ±SEM (n=5). There is a significant change of PSF expression across gestation (P=0.03). (C) Myometrial tissue was also collected during and after pregnancy from rats (n-4 at each time point). Total protein was extracted and subjected to Western blotting using a PR specific antibody to assess PR expression levels. Membranes were then stripped and western blotted with the anti-calponin antibody as a loading control. The intensity of PR protein bands was quantified by densitometry, and normalized by calponin. The bars represent mean ±SEM (n=4).

FIG. 10 is a graph showing effect of PSF-A on DHT-induced AR transactivation in PC-3(AR)2 cells.

FIG. 11 is a graph showing effect of PSF-F on DHT-induced AR transactivation in PC-3(AR)2 cells.

FIG. 12 is a blot showing PSF-A & AR Stability in PC-3(AR)₂ cells.

FIG. 13 is a graph showing PSF inhibits glucocorticoid receptor transactivation in SHM cells

### DETAILED DESCRIPTION OF THE INVENTION

In accordance with the present invention there may be employed conventional biochemistry, enzymology, molecular biology, microbiology, and recombinant DNA techniques within the skill of the art. Such techniques are explained fully in the literature. See for example, Sambrook et al, Molecular Cloning: A Laboratory Manual, Third Edition (2001) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%,5-50%, or 10-40%, preferably 10-20%, more preferably 10% or 15%, of the number to which reference is being made.

A “PSF Polypeptide” refers to a polypeptide comprising a RNA recognition motif II (RRMII) which interacts with a PR Polypeptide. A PSF Polypeptide can be a polypyrimidine tract-binding protein-associated splicing factor [Patton, J. G., et al., J. Genes Dev. 7 (3), 393-406 (1993)] including human PSF (GenBank NP_{—}005057 and P23246), and mouse PSF (GenBank NP 076092, SEQ ID NO. 19). Other polypeptides containing RRMII motif sequences may be identified with a protein homology search, for example by searching available databases such as GenBank or SwissProt and various search algorithms and/or programs may be used including FASTA, BLAST (available as a part of the GCG sequence analysis package, University of Wisconsin, Madison, Wis.), or ENTREZ (National Center for Biotechnology Information, National Library of Medicine, National Institutes of Health, Bethesda, Md.). A “PSF Polypeptide” includes the wild type polypeptide, or part thereof, or isoforms, variants, homologs, or salts of such a polypeptide. The term includes a polypeptide comprising the sequence of Accession Nos. NP 005057, NP 076092, AAH04534, AAH27708, AAH51192, CAA34747, CAA50283, or P23246, or SEQ ID NOs. 1, 2, 3, 4, 5, 6, or 21.

A “RNA recognition motif II” or “RRMII” refers to a polypeptide comprising or consisting essentially of a motif that is involved in RNA binding. A RRMII structure consists of four strands and two helices arranged in an alpha/beta sandwich, with a third helix present during RNA binding in some cases. A RRMII generally comprises a region of about 80 amino acids containing several well conserved sequences some of which cluster into two short submotifs, RNP-1 (octamer) and RNP-2 (hexamer). (See Birney et al, Nucleic Acids Res, 1993, 21(25):5803-5816 and references cited therein). A RRMII of a PSF Polypeptide may comprise or consist of amino acids 370 to 450 or 371 to 452 of SEQ ID NO. 1.

“Steroid receptor” refers to a member of a family of nuclear transcription factors which are receptors for hormones of the steroid family, including without limitation androgen, glucocorticoid, and progesterone. The present invention has particular applications in respect to the progesterone receptor (PR), androgen receptor (AR), and glucocorticoid receptor (GR), preferably the progesterone receptor.

A “progesterone receptor”, “PR”, or “PR Polypeptide” refers to a member of the steroid receptor superfamily that mediates the physiologic effects of progesterone. A PR Polypeptide comprises a DBD domain, AF1 domain, AF2 domain, and/or AF3 domain, preferably a DBD domain and/or an AF3 domain. A PR Polypeptide is capable of interacting with a PSF Polypeptide. The terms include the wild type polypeptide, or part thereof, or isoforms, variants, homologs, or salts of such a polypeptide. A PR Polypeptide includes the A isoform (SEQ ID NO. 10 with amino acids 1 to 164 missing) and 1 isoform (SEQ ID NO .10) of a progesterone receptor, and parts thereof including a domain or motif thereof, in particular an AF3 and/or DNA binding domain [See U.S. Pat. No. 5,439,796, Beato, M., Cell 5:335-344 (1989) Green et al., Nature 328:134-139 (1986); Hollenberg et al., Nature 318:635-641 (1985); Arriza et al., Science 237.:268-275 (1987); Misraki et al., Biochem. Biophys. Res. Comm. 143:740-748 (1987); Lubahn et al., Science 240:327-330 (1988); Chang et al., Science 240:324-326 (1988)]. In particular, the terms include the polypeptides comprising the sequences of Accession Nos. NP 000917, AAS00096, BAB91074, BAC06585, BAC11011, BAC11012, BAC11013, AAD01587, AAQ96833, AAQ96834, AAA60081, CAA36018, or P06401, or SEQ ID NOs. 10, 11, 12, 13, 14, or 15.

“DBD” or “DNA binding domain” refers to a polypeptide comprising or consisting of a consensus sequence of amino acids that recognize specific DNA bases near the start of transcription. The core sequence of a DBD domain is highly conserved among nuclear hormone receptors. It has over 40% amino acid identity over a 67-residue region (Rastinejad et al. 2000, EMBO J 19: 1045-1054.). The overall architecture of known DBD core structures is very similar (Khorasanizadeh & Rastinejad 2001, Trends Biochem Sci 26: 384-390), and is composed of two zinc-finger motifs, each containing four highly conserved cysteine molecules which coordinate the binding of a zinc atom. Zinc atoms and cysteine residues are necessary for maintaining a three dimensional structure whose core is composed of two helices (helix I and II) oriented at approximately right angles to each other. A DBD of a PR Polypeptide may comprise or consist of amino acids 567-587 of SEQ ID NO. 10.

An “activation function” or “AF” refers to a region of 30-100 amino acids in length classified by sequence similarity or the presence of predominant amino acids: acidic, glutamine-, or proline-rich. A PR Polypeptide comprises one or more of a constitutive activation domain AF1 in the N-terminus, a hormone-dependent AF2 in the ligand-binding domain (McKenna et al, 1999 Endocrine Reviews 20:321-344), and a N-terminal transcriptional modulatory domain AF3 (Sartorius et al, Molecular Endocrinology 8:1447-1360).

The term “wild type” refers to a polypeptide having a primary amino acid sequence that is identical with a native polypeptide (for example, the human polypeptide). The term specifically encompasses naturally occurring truncated or secreted forms of a polypeptide, polypeptide variants including naturally occurring variant forms (e.g., alternatively spliced forms or splice variants), and naturally occurring allelic variants. Such wild type or native-sequence polypeptides can be isolated from nature or can be produced by recombinant or synthetic means.

The term “variant” refers to a naturally occurring polypeptide that differs from a wild-type sequence. A variant may be found within the same species (i.e. if there is more than one isoform of the protein) or may be found within a different species. Preferably, the variant has at least 40%, 50%, 60%, 70%, 80%, 90%, 95%, or 99% sequence identity with the wild type sequence. Preferably, the variant has 20 mutations or less over the whole wild-type sequence. More preferably, the variant has 10 mutations or less, most preferably 5 mutations or less over the whole wild-type sequence. Variants include, for instance, polypeptides wherein one or more amino acid residues are added to, or deleted from, the N- or C-terminus of the full-length or mature sequences of SEQ ID NOs 1 to 6 and 10 to 15, and 21 including variants from other species, but excludes a native-sequence polypeptide.

A naturally occurring allelic variant may contain conservative amino acid substitutions from the native polypeptide sequence or it may contain a substitution of an amino acid from a corresponding position in a PSF Polypeptide homolog or PR Polypeptide homolog, for example, a murine PSF Polypeptide or PR Polypeptide.

An allelic variant may also be created by introducing substitutions, additions, or deletions into a nucleic acid encoding a wild type polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

The tern “part” indicates that the polypeptide comprises a fraction of the wild-type amino acid sequence. It may comprise one or more large contiguous sections of sequence or a plurality of small sections. The polypeptide may also comprise other elements of sequence, for example, it may be a chimeric polypeptide or fusion protein with another protein (such as one which aids isolation or crystallization of the polypeptide). Preferably the polypeptide comprises at least 50%, more preferably at least 65%, most preferably at least 80% of the wild-type sequence. A part of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A part of a PSF Polypeptide includes a binding domain of the polypeptide that interacts with a PR Polypeptide including a domain thereof (e.g. DBD domain and/or AF3 domain). For example a part of the PSF Polypeptide includes a RRMII domain, a polypeptide consisting of amino acids 1-150, amino acids 290-370, or amino acids 1-662 of a wild type PSF Polypeptide (e.g. SEQ ID NO. 1).

A part of a PR Polypeptide includes a binding domain of the polypeptide that interacts with a PSF Polypeptide. For example, a part of a PR Polypeptide includes a DBD domain, a AF3 domain, a polypeptide consisting of amino acids 1-164 of wild type progesterone receptor (e.g. SEQ ID NO. 10), 456-650 of wild type progesterone receptor (including the AF1 domain and DBD), amino acids 567-587, or amino acids 556 to 933 of wild type progesterone receptor (e.g. SEQ ID NO. 10) (including a DBD domain and AF2 domain).

The term “homolog” means a polypeptide having a degree of homology with the wild-type amino acid sequence, particularly substantial homology. The term “homology” refers to a degree of complementarity. There may be partial homology or complete homology. In aspects of the invention a PSF Polypeptide or PR Polypeptide has substantial homology to a wild type protein. A sequence that has “substantial homology” refers to a partially complementary sequence that at least partially inhibits an identical sequence from hybridizing to a target nucleic acid. Inhibition of hybridization of a completely complementary sequence to the target sequence may be examined using a hybridization assay (e.g. Southern or northern blot, solution hybridization, etc.) under conditions of reduced stringency. A sequence has substantial homology or a hybridization probe will compete for and inhibit the binding of a completely homologous sequence to the target sequence under conditions of reduced stringency. However, conditions of reduced stringency can be such that non-specific binding is permitted, as reduced stringency conditions require that the binding of two sequences to one another be a specific (i.e., a selective) interaction. The absence of non-specific binding maybe tested using a second target sequence which lacks even a partial degree of complementarity (e.g., less than about 30% homology or identity). A sequence or probe with substantial homology will not hybridize to the second non-complementary target sequence in the absence of non-specific binding.

A sequence of a PSF Polypeptide or PR Polypeptide contemplated by the invention may have at least 30%, 40%, 50%, 60%, 65%,70%, 75%, 80%, 85%, 90%, 95%, or 99% identity. The phrases “percent identity” or “% identity” refer to the percentage comparison of two or more amino acid or nucleic acid sequences. Percent identity can be determined electronically using for example the MegAlign program (DNASTAR, Inc., Madison Wis.). The MegAlign program can create alignments between two or more sequences according to different methods, e.g., the Clustal method. (See, e.g., Higgins, D. G. and P. M. Sharp (1988) Gene 73:237-244.) Percent identity between nucleic acid sequences can also be determined by other methods known in the art, e.g., the Jotun Hein method. (See, e.g., Hein, J. (1990) Methods Enzymol. 183:626-645.) PSF Polypeptides and PR Polypeptides include chimeric or fusion polypeptides. “Chimeric polypeptide” and “fusion polypeptide” are used interchangeably herein and comprise all or part (preferably biologically active) of a PSF polypeptide or PR Polypeptide operably linked to a heterologous polypeptide (i.e., a polypeptide other than a PSF Polypeptide or PR Polypeptide, respectively). Chimeric polypeptides are recombinant in the sense that the various components are from different sources, and as such are not found together in nature (i.e. are heterologous). Within the fusion protein, the term “operably linked” is intended to indicate that a PSF Polypeptide or PR Polypeptide and the heterologous polypeptide are fused in-frame to each other. The heterologous polypeptide can be fused to the N-terminus or C-terminus of a PSF polypeptide or PR Polypeptide. A useful fusion polypeptide is a GST fusion polypeptide in which a PSF polypeptide is fused to the C-terminus of GST sequences. Another example of a fusion polypeptide is an immunoglobulin fusion polypeptide in which all or part of a PSF polypeptide or PR Polypeptide is fused to sequences derived from a member of the immunoglobulin protein family. Chimeric and fusion polypeptides can be produced by standard recombinant DNA techniques.

The terms also include chimeric polypeptides comprising a PSF Polypeptide or PR Polypeptide fused to, or integrated into a targeting domain capable of directing the chimeric polypeptide to a desired cellular component or cell type or tissue. The chimeric polypeptides may also contain additional amino acid sequences or domains. A targeting domain can be a membrane spanning domain, a membrane binding domain, or a sequence directing the PSF Polypeptide or PR Polypeptide to associate with, for example, vesicles or with the nucleus. The targeting domain can target a PSF Polypeptide or PR Polypeptide to a particular cell type or tissue. For example, the targeting domain can be a cell surface ligand or an antibody against cell surface antigens of a target tissue (e.g. tumor antigens). A targeting domain may target a PSF Polypeptide to a cellular component.

Polypeptides and chimeric polypeptides disclosed herein maybe converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids. Pharmaceutical salts may be used in the compositions and methods described herein.

A “PSF Complex” refers to a molecule comprising or consisting essentially of a PSF Polypeptide interacting with a steroid receptor. A PSF complex is preferably an isolated complex and it may be a fusion polypeptide or chimeric polpeptide. A complex may comprise only the binding domains of the interacting molecules and such other flanking sequences as are necessary to maintain the activity of the complex. In particular aspects of the invention a PSF Complex comprises a GR, AR or preferably a PR Polypeptide (PSF-PR Complex).

A “PSF-PR Complex” refers to a molecule comprising or consisting essentially of a PSF Polypeptide interacting with a PR Polypeptide. A PSF-PR complex may be a fusion polypeptide or chimeric polpeptide. A complex may comprise only the binding domains of the interacting molecules and such other flanking sequences as are necessary to maintain the activity of the complex. In an aspect of the invention a complex is provided comprising a PSF Polypeptide of SEQ ID NO.1 or part thereof, interacting with a PR Polypeptide of SEQ ID NO.10, or a part thereof. In particular, a PSF-PR Complex is provided comprising a PSF Polypeptide of SEQ ID NO.1 or RRMII thereof, interacting with a progesterone receptor isoform A (SEQ ID NO. 1 with amino acids 1 to 164 missing), or a DBD thereof. A PSF-PR complex can comprise the RRMII domain of a PSF Polypeptide interacting with a DBD domain of a PR Polypeptide, or a RRMII domain of a PSF Polypeptide interacting with an AF3 domain of a PR Polypeptide.

The terms “interact”, “interaction”, or “interacting” refer to any physical association between molecules including protein-protein interactions. The term preferably refers to a stable association between two molecules due to, for example, electrostatic, hydrophobic, ionic and/or hydrogen-bond interactions under physiological conditions. Certain interacting or associated molecules interact only after one or more of them has been stimulated. An interaction between polypeptides or parts thereof, and other cellular molecules may be either direct or indirect. Various methods known in the art can be used to measure the level of an interaction. For example, the strength of covalent bonds may be measured in terms of the energy required to break a certain number of bonds.

“PSF Polynucleotides” refers to polynucleotides encoding PSF Polypeptides including native-sequence polypeptides, polypeptide variants including a part of a PSF Polypeptide, an isoform, precursor, complex, a chimeric polypeptide, or modified forms and derivatives of the polypeptides. A PSF Polynucleotide can be a polynucleotide comprising or consisting of a sequence of Accession Nos. NM_{—}005066, BC004534, BC027708, BC027717, BC051192, X16850, S56626, or X70944, or SEQ ID NOs. 7, 8, or 9.

PSF Polynucleotides include complementary nucleic acid sequences, and nucleic acids that are at least about 40%, 45%, 50%, 55%, 60%, 65%, 70%, 75%, 80%, 85%, 90%, 95%, 97%, 98%, or 99% identity.

PSF Polynucleotides also include sequences that differ from a native sequence due to degeneracy in the genetic code. As one example, DNA sequence polymorphisms within the nucleotide sequence of a PSF Polynucleotide may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Polynucleotides also include nucleic acids that hybridize under stringent conditions, preferably high stringency conditions to a PSF Polynucleotide. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

PSF Polynucleotides also include truncated nucleic acids or nucleic acid fragments and variant forms of the nucleic acids that arise by alternative splicing of an mRNA corresponding to a DNA.

PSF Polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term refers to antisense polynucleotides (e.g. mRNA or DNA strand in the reverse orientation to sense PSF Polynucleotides).

The terms “sample”, “biological sample”, and the like mean a material known or suspected of expressing or containing a PSF Polypeptide, PSF Polynucleotide, and/or PSF Complex. The test sample can be used directly as obtained from the source or following a pretreatment to modify the character of the sample. The sample can be derived from any biological source, such as tissues (e.g. myometrial tissue), extracts, or cell cultures, including cells, cell lysates, conditioned medium from fetal or maternal cells, and physiological fluids, such as, for example, whole blood, plasma, serum, saliva, ocular lens fluid, cerebral spinal fluid, sweat, urine, milk, ascites fluid, amniotic fluid, vaginal fluid, synovial fluid, peritoneal fluid and the like.

A sample can be treated prior to use, such as preparing plasma from blood, diluting viscous fluids, and the like. Methods of treatment can involve filtration, distillation, extraction, concentration, inactivation of interfering components, the addition of reagents, and the like. Polypeptides and polynucleotides may be isolated from the samples and utilized in the methods of the invention.

In embodiments of the invention the sample is a mammalian sample, preferably human sample. In another embodiment the sample is a physiological fluid. In a particular embodiment, the sample is serum, amniotic fluid or vaginal fluid.

The terms “subject”, “individual”, or “patient” refer to an animal including a warm-blooded animal such as a mammal, which is afflicted with or suspected of having or being pre-disposed to a condition or disease described herein. Mammal includes without limitation any members of the Mammalia. In general, the terms refer to a human. The terms also include animals bred for food, pets, or sports, including domestic animals such as horses, cows, sheep, poultry, fish, pigs, and goats, and cats, dogs, and zoo animals, apes (e.g. gorilla or chimpanzee), and rodents such as rats and mice. The methods herein for use on subjects/individuals/patients contemplate prophylactic as well as curative use. Typical subjects for treatment include persons susceptible to, suffering from or that have suffered a condition or disease described herein.

The term “agonist” of a polypeptide of interest, for example, a PSF Polypeptide, is used in its broadest sense. An agonist can include any agent that results in activation, enhancement, or alteration of the presence of (e.g. an increase in the presence of) a PSF Polypeptide or PSF Complex. An agonist may interact with a polypeptide and maintain or increase the activity of the polypeptide with which it interacts. Agonists may include proteins, peptides, nucleic acids, carbohydrates, or any other molecules that bind to a PSF Polypeptide, a PSF complex, or a PR Polypeptide. Agonists also include molecules derived from, or that bind to, an active site or domain of a PSF Polypeptide or a PR Polypeptide. Peptide mimetics, synthetic molecules with physical structures designed to mimic structural features of particular peptides, may serve as agonists. The stimulation may be direct, or indirect, or by a competitive or non-competitive mechanism. .

In an embodiment, an agonist is a molecule that stimulates, enhances, or increases the activity of a PSF Polypeptide or PSF Complex, or the binding of a PSF Polypeptide and a PR Polypeptide for induction of labor, in particular in over term pregnancy. An agonist may control or alter PSF Polypeptide, PR Polypeptide, or PSF Complex availability or levels useful for induction of labor in over term pregnancy.

The term “antagonist” or “antagonizing” is used in its broadest sense. Antagonism can include any mechanism or treatment that results in inhibition, inactivation, blocking or reduction or alteration of the presence of a PSF Polypeptide or PSF Complex. An antagonist may interact with a PSF Polypeptide or PSF Complex but not maintain the activity of the polypeptide with which it interacts or have an effect that is opposite to that of a PSF Polypeptide or PSF Complex. Antagonists may include proteins, peptides, nucleic acids, carbohydrates, or any other molecules that bind to a PSF Polypeptide, PSF complex, or PR Polypeptide. Antagonists also include molecules derived from, or that bind to, an active site or domain of a PSF Polypeptide or a PR Polypeptide. Peptide mimetics, synthetic molecules with physical structures designed to mimic structural features of particular peptides, and antibodies may serve as antagonists. The inhibition may be direct, or indirect, or by a competitive or non-competitive mechanism. Examples of antagonists are binding agents for a PSF polypeptide such as antibodies specific for a PSF Polypeptide, and inhibitors of polynucleotides encoding a PSF Polypeptide (e.g. antisense).

In an embodiment, an antagonist is a molecule that inhibits the activity of a PSF Polypeptide or a PSF Complex, or the binding of a PSF Polypeptide and PSF Complex, in causing pre-term labor. An antagonist may control or alter PSF Polypeptide, PR Polypeptide or PSF Complex availability or levels useful for control and inhibition of pre-term labor.

An agonist or antagonist may be a peptide derived or optimized from a motif or domain of a PSF Polypeptide or a PR Polypeptide. Peptides include analogs, and/or truncations of the peptides, which may include, but are not limited to the peptides containing one or more amino acid insertions, additions, or deletions, or both. Analogs of a peptide can exhibit the activity characteristic of the peptide, and may further possess additional advantageous features such as increased bioavailability, stability, or reduced host immune recognition.

“Peptide mimetics” or “peptidomimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or agonist or antagonist (i.e. enhancer or inhibitor). Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a motif, peptide, or agonist or antagonist (i.e. enhancer or inhibitor).

The term “isolated” in reference to a polypeptide or complex herein refers to a polypeptide or complex substantially free of cellular material, or culture medium when produced by recombinant DNA techniques, or chemical reactants, or other chemicals when chemically synthesized. An isolated polypeptide is also preferably free of sequences which naturally flank the polypeptide.

“PR Polypeptide degradation”, “degradation of a PR Polypeptide”, or “PSF Polypeptide mediated degradation of a PR Polypeptide” refers to degradation of a PR Polypeptide through cellular processes such as the proteosome pathway. The degradation of a PR Polypeptide is preferably associated with the interaction of a RRMII domain and a DBD domain.

“Steroid receptor signal transduction pathway” or “steroid receptor signaling pathway” refers to the sequence of events that involves the transmission of a message from an extracellular protein (e.g. steroid hormone) to the cytoplasm through the cell membrane. Signal transduction pathways contemplated herein include pathways involving a steroid receptor or parts thereof (e.g. DBD domain and/or AF3 domain), optionally a PSF Polypeptide or parts thereof (e.g. RRMII), or a PSF Complex. The amount and intensity of a given signal in a signal transduction pathway can be measured using conventional methods. For example, the concentration and localization of various proteins and complexes in a signal transduction pathway can be measured, conformational changes that are involved in the transmission of a signal may be observed using circular dichroism and fluorescence studies, and various physiological processes associated with a signal transduction pathway may be detected.

“Progesterone receptor signal transduction pathway” or “progesterone receptor signaling pathway” refers to a steroid receptor signal transduction pathway involving a PR Polypeptide or parts thereof (e.g. DBD domain and/or AF3 domain), optionally a PSF Polypeptide or parts thereof (e.g. RRMII), or a PS-PR complex.

“Condition mediated by a steroid receptor” refers to a condition or disease in which a seriod receptor, a PSF Polypeptide, a PSF Polynucleotide, PSF Complex, and/or an interaction of a steroid receptor and PSF Polypeptide play a role. The term includes conditions characterized by an abnormality in a steroid receptor signal transduction pathway, degradation of a steroid receptor, steroid receptor transactivation, or a process mediated by a PSF Polypeptide, PSF Polynucleotide, PSF Complex and/or a steroid receptor.

“Condition mediated by a progesterone receptor” refers to a condition or disease in which a PSF Polypeptide, PSF Polynucleotide, PR Polypeptide, PSF-PR Complex, or an interaction of a PR Polypeptide and PSF Polypeptide play a role. The term includes conditions characterized by an abnormality in a progesterone receptor signal transduction pathway, degradation of a PR Polypeptide, progesterone receptor transactivation, or a process mediated by a PSF Polypeptide, PSF Polynucleotide, PSF Complex and/or PR Polypeptide.

The invention has particular application for the following: enhancement of bone formation in bone weakening diseases for the treatment or prevention of osteopenia or osteoporosis; enhancement of fracture healing; use as a female contraceptive agent; prevention of endometrial implantation; induction of labor; treatment of luteal deficiency; enhanced recognition and maintanence of pregnancy; counteracting of preeclampsia, eclampsia of pregnancy, and preterm labor; treatment of infertility, including promotion of spermatogenesis, induction of the acrosome reaction, maturation of oocytes, or in vitro fertilization of oocytes; treatment of dysmenorrhea; treatment of dysfunctional uterine bleeding; treatment of ovarian hyperandrogynism; treatment of ovarian hyperaldosteronism; alleviation of premenstrual syndrome and of premenstrual tension; alleviation of perimenstrual behavior disorders; treatment of climeracteric disturbance, including menopause transition, mood changes, sleep disturbance, and vaginal dryness; enhancement of female sexual receptivity and male sexual receptivity; treatment of post menopausal urinary incontinence; improvement of sensory and motor functions; improvement of short term memory; alleviation of postpartum depression; treatment of genital atrophy; prevention of post-surgical adhesion formation; regulation of uterine immune function; prevention of myocardial infarction; hormone replacement; treatment of cancers, including hormone mediated cancers, such as breast cancer, uterine cancer, ovarian cancer, prostate cancer, and endometrial cancer; treatment of endometriosis; treatment of uterine fibroids; treatment of hirsutism; and inhibition of hair growth.

The invention has particular application in modulating the onset of labor, in particular in the treatment and prevention of pre-term labor.

“Pre-term labor” refers to the premature onset of labor resulting in expulsion from the uterus of an infant before the normal end of gestation (i.e. pre-term birth or delivery), or more particularly, onset of labor with effacement and dilation of the cervix before the 37th week of gestation. It may or may not be associated with vaginal bleeding or rupture of membranes. Pre-term labor may be related to factors including without limitation infection (eg, bacterial vaginosis [BV], sexually transmitted diseases [STDs], urinary tract infections, chorioamnionitis), uterine distention (eg, multiple gestation, polyhydramnios), uterine distortion (eg, müllerian duct abnormalities, fibroid uterus), compromised structural support of the cervix (e.g., incompetent cervix, previous cone biopsy or loop electrosurgical excision procedure [LEEP]), abruptio placentae, uteroplacental insufficiency (eg, hypertension, insulin-dependent diabetes, drug abuse, smoking, alcohol consumption), stress either indirectly by associated risk behaviors or by direct mechanisms including fetal stress.

The term “mediate” or “mediated” refers to an affect or influence, frequently indirectly or via some intervening action. Thus, for example, conditions mediated by a PR Polypeptide are those in which a PR Polypeptide plays a role.

The term “modulate” means affect or influence, for example, the amount, degree or proportion. Thus, compounds that “modulate” a PSF Polypeptide affect the activity, either positively or negatively, of that polypeptide. The term may be used to refer to the activity of an agonist, partial agonist or antagonist of a polypeptide. The term also may be used to refer to the effect that a compound has on a physical and/or physiological condition of an individual. For example, certain agonists or antagonists of the present invention may be used to modulate labor in an individual. That is, certain compounds of this invention may be used to induce labor in an individual, while other compounds of this invention may be used to stop or delay labor.

“Binding agent” refers to a substance such as a polypeptide or antibody that specifically binds to one or more PSF Polypeptide. A substance “specifically binds” to one or more PSF Polypeptide if is reacts at a detectable level with one or more PSF Polypeptide, and does not react detectably with peptides containing an unrelated or different sequence. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises one or more PSF Polypeptide sequence, a peptide variant thereof, or a non-peptide mimetic of such a sequence.

An aptamer includes a DNA or RNA molecule that binds to nucleic acids and proteins. An aptamer that binds to a protein (or binding domain) or a PSF Polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies for use in the present invention include but are not limited to monoclonal antibodies, polyclonal antibodies, antibody fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), recombinantly produced binding partners, chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant PSF Polypeptides may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for a PSF Polypeptide may also be obtained from scientific or commercial sources. In an embodiment of the invention, antibodies are reactive against a PSF Polypeptide if they bind with a Kₐ of greater than or equal to 10⁻⁷ M. Binding partners may be constructed utilizing recombinant DNA techniques to incorporate the variable regions of a gene which encodes a specifically binding antibody (See Bird et al., Science 242:423-426, 1988).

A PSF Polypeptide, PSF Polynucleotide, PSF Complex, and binding agents may be labeled using conventional methods with various detectable substances. A “detectable substance” is a substance that is capable of producing, either directly or indirectly, a detectable signal and allows for detection and optionally isolation of a polypeptide, polynucleotide or complex. In the methods of the invention, a detectable substance is preferably selected that does not interfere with the interaction of a binding agent and its binding partner. Examples of detectable substances include, but are not limited to, the following: radioisotopes (e.g., ³H, ¹⁴C, ³²p, ³⁵S, ¹²⁵I, ¹³¹I), fluorescent labels (e.g., FITC, rhodamine, lanthanide phosphors umbelliferone, fluorescein, fluorescein isothiocyanate, rhodamine, dichlorotriazinylamine fluorescein, dansyl chloride or phycoerythrin), luminescent labels such as luminol; enzymatic labels (e.g., horseradish peroxidase, beta-galactosidase, luciferase, alkaline phosphatase, malate dehydrogenase, ribonuclease, urease, catalase, glucose-6-phosphate, staphylococcal nuclease, delta-5-steroid isomerase, yeast alcohol dehydrogenase, alpha-glycerophosphate, triose phosphate isomerase, asparaginase, glucose oxidaseacetylcholinesterase), bioluminescent labels (e.g. luciferin, luciferase and aequorin), biotinyl groups (which can be detected by marked avidin e.g., streptavidin containing a fluorescent marlcer or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags) including without limitation enzymes, fluorescent materials, luminescent materials and radioactive materials. Suitable enzymes, fluorescent materials, luminescent materials, and radioactive material are well known to the skilled artisan. In some embodiments, detectable substances are attached via spacer arms of various lengths to reduce potential steric hinderance. In some applications binding agents such as antibodies may be coupled to electron dense substances, such as ferritin or colloidal gold, which are readily visualized by electron microscopy.

The terms “administering” or “administration” refer to the process by which an effective amount or therapeutically effective amount of compounds or a composition contemplated herein are delivered to a patient for treatment purposes. Compounds and compositions are administered in accordance with good medical or veterinary practices taking into account the patient's clinical condition, the site and method of administration, dosage, patient age, sex, body weight, and other factors known to physicians.

“Micro-array” and “array,” refer to nucleic acid or nucleotide arrays or protein or peptide arrays that can be used to detect PSF Polynucleotides, PSF Polypeptides, or PSF Complexes, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known [(see for example, Zhu et al., Science 293:2101 (2001)].

Methods for Identifying Modulators

The invention provides methods to screen or identify modulators (i.e. agonists and antagonists) of a PSF Polypeptide, a PSF Complex, or the interaction of a PSF Polypeptide and a steroid receptor, modulators identified by such methods, and, methods and compositions using such modulators.

Therefore, the invention provides a method of selecting a substance that modulates a steroid receptor in particular a PR Polypeptide; a PSF Polypeptide; a PSF Complex, in particular a PSF-PR Complex; a process mediated by a steroid receptor, in particular a PR Polypeptide; PSF Polypeptide mediated degradation of a steroid receptor, in particular a PR Polypeptide; a steroid receptor signaling transduction pathway, in particular a progesterone receptor signal transduction pathway; a condition mediated by a steroid receptor, in particular mediated by a PR Polypeptide; steroid receptor transactivation, in particular PR transactivation, and/or inhibits or potentiates the interaction of a steroid receptor and a PSF Polypeptide, comprising assaying for a substance that inhibits or stimulates (i.e. is an agonist or antagonist of) a PSF Polypeptide, a PSF Polynucleotide, or PSF Complex.

In an aspect, the invention provides a method of selecting a substance that modulates a PSF Polypeptide, a PR Polypeptide a process mediated by a PR Polypeptide, PSF mediated degradation of a PR Polypeptide, a progesterone receptor signal transduction pathway, PR Polypeptide transactivation, and/or a condition mediated by a progesterone receptor; comprising assaying for a substance that inhibits or stimulates (i.e. is an agonist or antagonist of) a PSF Polypeptide, a PSF Polynucleotide, or PSF Complex.

Substances that modulate a steroid receptor, in particular a PR Polyp eptide; a PSF Polypeptide; a PSF Complex, in particular a PSF-PR Complex; a process mediated by a steroid receptor, in particular a PR Polypeptide; PSF Polypeptide mediated degradation of a steroid receptor, in particular a PR Polypeptide; a steroid receptor signaling transduction pathway, in particular a progesterone receptor signal transduction pathway; a condition mediated by a steroid receptor, in particular mediated by a PR Polypeptide; steroid receptor transactivation, in particular PR transactivation, and/or inhibits or potentiates the interaction of a steroid receptor and a PSF Polypeptide, can be selected by assaying for a substance that inhibits or stimulates the activity of a PSF Polypeptide.

In an aspect, the invention relates to a method of selecting a substance that modulates or regulates the onset of labor comprising assaying for a substance that inhibits or stimulates (i.e. is an agonist or antagonist of) a PSF Polypeptide. The substances may be used in the methods of the invention to modulate or regulate the onset of labor.

Methods are contemplated for identifying substances that interact with or bind to a PSF Polypeptide, or PSF complex, or bind to other proteins that interact with the molecules or complex, to compounds that interfere with, or enhance the interaction of molecules through a PSF Polypeptide, or other proteins that interact with the molecules.

Substances that modulate the activity of a PSF Polypeptide or PSF Complex can be identified based on their ability to interact with or bind to a PSF Polypeptide, a molecule derived from a PSF Polypeptide, or a PSF Complex. Therefore, the invention also provides methods for identifying substances which bind a PSF Polypeptide, a molecule derived from a PSF Polypeptide, or a PSF Complex. Substances identified using the methods of the invention may be isolated, cloned and sequenced using conventional techniques.

In an aspect, substances which can bind with a PSF Polypeptide, or a molecule in a PSF Complex, may be identified by reacting a PSF Polypeptide, or molecule in a PSF Complex with at least one test substance which potentially interacts with or binds to a PSF Polypeptide, or molecule under conditions which permit the formation of complexes between the substance and PSF Polypeptide, or molecule, and removing and/or detecting binding. Binding can be detected by assaying for complexes. The detection of complexes indicates the substance binds to the PSF Polypeptide, or molecule. The complexes can be detected by assaying for substance-molecule complexes, for free substance, or for non-complexed PSF Polypeptide or molecules, or activation of the PSF Polypeptides or PSF Complex or a steroid receptor. Conditions which permit the formation of complexes maybe selected having regard to factors such as the nature and amounts of the substance and the PSF Polypeptide, or molecule. The invention also contemplates methods for identifying substances that bind to other proteins that interact with a PSF Polypeptide.

A substance that modulates a steroid receptor, in particular a PR Polyp eptide; a PSF Polypeptide; a PSF Complex, in particular a PSF-PR Complex; a process mediated by a steroid receptor, in particular a PR Polypeptide; PSF Polypeptide mediated degradation of a steroid receptor, in particular a PR Polypeptide; a steroid receptor signaling transduction pathway, in particular a progesterone receptor signal transduction pathway; a condition mediated by a steroid receptor, in particular mediated by a PR Polypeptide; and/or steroid receptor transactivation, in particular PR transactivation, can also be identified based on its ability to specifically interfere or stimulate the interaction of a PSF Polypeptide and steroid receptor, in particular PR, GR or AR.

In an aspect a substance that modulates a PR Polypeptide, a PSF Polypeptide, a process mediated by a PR Polypeptide, degradation of PR Polypeptide, a progesterone receptor signal transduction pathway, PR Polypeptide transactivation, and/or a condition mediated by a PR Polypeptide, in particular regulates the onset of labor, can also be identified based on its ability to specifically interfere or stimulate the interaction of a PSF Polypeptide and progesterone receptor.

The association or interaction between a PSF Polypeptide and a steroid receptor may be promoted or enhanced either by increasing production of a PSF Polypeptide, or by increasing expression of a PSF Polypeptide, or by promoting interaction of a PSF Polypeptide or by prolonging the duration of the association or interaction. The association or interaction between a PSF Polypeptide and a steroid receptor may be disrupted or reduced by preventing production of a PSF Polypeptide, or by preventing expression of a PSF Polypeptide, or by preventing interaction of a PSF Polypeptide, or interfering with the interaction. A method may include measuring or detecting various properties including the level of signal transduction and the level of interaction between a PSF Polypeptide and a steroid receptor. Depending upon the type of interaction present, various methods maybe used to measure the level of interaction. For example, the strengths of covalent bonds are often measured in terms of the energy required to break a certain number of bonds (i.e., kcal/mol). Non-covalent interactions are often described as above, and also in terms of the distance between the interacting molecules. Indirect interactions maybe described in a number of ways, including the number of intermediary agents involved, or the degree of control exercised over the PSF Polypeptide relative to the control exercised over the steroid receptor.

The invention provides a method of testing an agent for its ability to affect the interaction between a PSF Polypeptide and a steroid receptor comprising (a) exposing an agent to a PSF Polypeptide and a steroid receptor for a sufficient time to allow the PSF Polypeptide and steroid receptor to interact; (b) removing non-bound agent; and (c) determining the presence of agent bound to the PSF Polypeptide and/or the steroid receptor thereby identifying an agent that affects the interaction.

In an aspect or the invention, an agent is tested for its ability to affect the interaction between a PSF Polypeptide and a PR Polypeptide comprising (a) exposing an agent to a PSF Polypeptide and a PR Polypeptide for a sufficient time to allow the PSF Polypeptide and PR Polypeptide to interact; (b) removing non-bound agent; and (c) determining the presence of agent bound to the PSF Polypeptide and/or the PR Polypeptide thereby identifying an agent that affects the interaction.

The invention also provides a method for evaluating a compound for its ability to modulate a steroid receptor; a PSF Polypeptide; a PSF Complex; a process mediated by a steroid receptor; PSF Polypeptide mediated degradation of a steroid receptor; a steroid receptor signaling transduction pathway; a condition mediated by a steroid receptor; and/or steroid receptor transactivation comprising:
(a) reacting a PSF Polypeptide or a part thereof that binds to a steroid receptor (e.g. RRMII) with a steroid receptor or a part thereof that binds to a PSF Polypeptide (e.g. AF3 and/or DBD), and a test substance; and
(b) comparing to a control in the absence of the test substance to determine the effect of the substance.

In an aspect, the invention provides a method for evaluating a compound for its ability to modulate or regulate a PSF Polypeptide, a PSF-PR Complex, a PR Polypeptide a process mediated by a PR Polypeptide, PSF mediated degradation of a PR Polypeptide, a progesterone receptor signal transduction pathway, PR Polypeptide transactivation, and/or a condition mediated by a progesterone receptor, in particular modulate or regulate the onset of labor, comprising the steps of:
(a) reacting a PSF Polypeptide or a part thereof that binds to a progesterone receptor (e.g. RRMII) with a progesterone receptor or a part thereof that binds to a PSF Polypeptide (e.g. AF3 and/or DBD), and a test substance; and
(b) comparing to a control in the absence of the test substance to determine the effect of the substance.

In particular, a method is provided for identifying a substance that modulates or regulates a PSF Polypeptide, a PR Complex, a PR Polypeptide a process mediated by a PR Polypeptide, PSF mediated degradation of a PR Polypeptide, a progesterone receptor signal transduction pathway, PR Polypeptide transactivation, and/or a condition mediated by a progesterone receptor, in particular modulates or regulates the onset of labor, comprising the steps of:
(a) reacting a PSF Polypeptide or a part thereof that binds to a progesterone receptor (e.g. RRMII) with a progesterone receptor or a part thereof that binds to a PSF Polypeptide (e.g. AF3 and/or DBD), and a test substance, under conditions which permit the formation of PSF complexes, and
(b) assaying for complexes, for free substance, for non-complexed PSF Polypeptide or PR Polypeptide, or for activation of PR Polypeptide.

The substance may stimulate or inhibit the interaction of a PSF Polypeptide or a part thereof that binds a steroid receptor (in particular a PR Polypeptide), and a steroid receptor (in particular a PR Polypeptide), or part that binds to a PSF Polypeptide.

The invention also provides a method for identifying antagonists and agonists of the interaction of a PSF Polypeptide and a steroid receptor, in particular a PR Polypeptide, comprising:
(a) providing a reaction mixture including a PSF Polypeptide and a steroid receptor, in particular a PR Polypeptide, or at least a portion of each which interact;
(b) contacting the reaction mixture with one or more test compounds;
(c) identifying compounds which inhibit the interaction of the PSF Polypeptide and steroid receptor, in particular a PR Polypeptide.

In an aspect the invention provides a method for evaluating a compound for its ability to modulate PSF mediated degradation of a steroid receptor, in particular degradation of a progesterone receptor through the proteosome pathway. For example, the compound may be a substance which binds to a PSF Polypeptide, or a substance which disrupts or promotes the interaction of molecules in a PSF Complex, in particular a PSF-PR Complex.

In another aspect the invention provides a method for screening an agent to be tested for an ability to modulate a steroid receptor signaling pathway, in particular a progesterone receptor signaling pathway, by testing for the ability of the agent to affect the interaction between a PSF Polypeptide and a steroid receptor, in particular a PR Polypeptide, wherein a complex formed by such interaction is part of the signal transduction pathway. The agent may bind to a PSF Polypeptide, or a substance which disrupts or promotes the interaction of molecules in a PSF Complex.

The invention contemplates a method for evaluating a compound for its ability to modulate the biological activity of a PSF Complex, by assaying for an agonist or antagonist (i.e. enhancer or inhibitor) of the binding of molecules in the complex through a binding domain, in particular a RRMII motif. A basic method for evaluating if a compound is an agonist or antagonist of the binding of molecules in a PSF complex, is to prepare a reaction mixture containing molecules and the substance, under conditions which permit the formation of complexes, in the presence of a test compound. The test compound may be initially added to the mixture, or may be added subsequent to the addition of molecules. Control reaction mixtures without the test compound or with a placebo are also prepared. The formation of complexes is detected and the formation of complexes in the control reaction but not in the reaction mixture indicates that the test compound interferes with the interaction of the molecules. The reactions may be carried out in the liquid phase or the molecules, or test compound may be immobilized as described herein. The ability of a compound to modulate the biological activity of a PSF Polypeptide, or PSF Complex of the invention may be tested by determining the biological effects on cells or organisms using techniques known in the art.

In the methods of the invention, complexes, free substance, or non-complexed molecules may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against the PSF Polypeptide, molecule or the substance, or labeled steroid receptor (e.g. PSF Polypeptide), or molecule, or a labeled substance may be utilized. The antibodies, motifs, binding partners, molecules, or substances may be labeled with a detectable substance as described above.

Activation of a steroid receptor, in particular a PR Polypeptide, may be assayed using conventional hormone response assays or transfection assays, for example two-hybrid systems and transactivation asssays described herein. Commercially available assays maybe used such as the GeneBLAzer® technology (Invitrogen, California, USA). Activation of a PSF Polypeptide maybe assayed using conventional orphosphorylation assays or transcription activation methods as discussed and exemplified herein.

A PSF Polypeptide, PSF Complex, steroid receptor, binding agent, substance, agent or compound used in a method of the invention may be insolubilized. For example, a polypeptide, binding partner, molecule, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling.

The invention also makes it possible to screen for antagonists that inhibit the effects of an agonist of an interaction of molecules in a PSF Complex of the invention. Thus, the invention may be used to assay for a compound that competes for the same binding site of a molecule in a PSF complex.

The invention also contemplates methods for identifying compounds that interact with or bind to proteins that interact with a molecule of a PSF Complex. Protein-protein interactions may be identified using conventional methods such as co-immunoprecipitation, crosslinking and co-purification through gradients or clromatographic columns. Methods may also be employed that result in the simultaneous identification of genes which encode proteins interacting with a molecule. These methods include probing expression libraries with labeled molecules. Additionally, x-ray crystallographic studies maybe used as a means of evaluating interactions with substances and molecules. For example, purified recombinant molecules in a PSF Complex when crystallized in a suitable form are amenable to detection of intra-molecular interactions by x-ray crystallography. Spectroscopy may also be used to detect interactions and in particular, Q-TOF instrumentation may be used. Two-hybrid systems may also be used to detect protein interactions in vivo. Protein clusters or pathways can be generated comprising the identified protein-protein interactions. (See, for example, Breitkreutz B J et al, Genome Biol. 2003;4(3):R22. Epub 2003 Feb. 27; Shannon, P et al, Genome Research 13:2498-2504, 2003; Sirava M et al; Bioinformatics. 2002 October; 18 Suppl 2:S219-S230; and Trost E et al, Bioinformatics. 2003 Apr. 12; 19(6):786-7 regarding software for creating biomolecular interaction networks.) In aspects of the invention, cell based assays are used to identify agonists and antagonists. In particular, a mammalian two-hybrid system or a cell based method for assaying transcriptional transactivation of steroid receptor promoters may be used to identify agonists and antagonists. For example, a cell based transactivation assay may involve introducing into cells (e.g. SHM cells) a steroid receptor, a PSF Polypeptide, a test compound and a steroid responsive promoter operably linked to a gene encoding a detectable substance (enzyme substrate), in the presence of a steroid, and determining the effect of the test substance by detecting the detectable substance. In such an assay, an agonist of a PSF Polypeptide will increase or enhance inhibition of steroid receptor transactivation of the promoter by the PSF Polypeptide, and an antagonist will inhibit or block the inhibitory effects of a PSF Polypeptide resulting in transactivation of the promoter.

Therefore, the invention provides a cell based assay for identifying a substance that modulates steroid receptor transactivation, comprising (a) introducing into cells a steroid receptor, a PSF Polypeptide, a test compound and a steroid responsive promoter operably linked to a gene encoding a detectable substance, in the presence of a steroid, and (b) assaying for an increase in inhibition of steroid receptor transactivation of the promoter by the PSF Polypeptide, or a decrease in inhibitory effects of a PSF Polypeptide resulting in transactivation of the promoter by detecting the detectable substance.

It will be appreciated that fusion polypeptides and recombinant fusion proteins may be used in the above-described methods. For example, a PSF Polypeptide fused to a glutathione-S-transferase may be used in the methods.

It will also be appreciated that the PSF Complexes may be reconstituted in vitro using recombinant molecules and the effect of a test substance may be evaluated in the reconstituted system.

Peptides derived from a PSF Polypeptide or steroid receptor, in particular a PR Polypeptide, may be used to identify lead compounds for drug development. The structure of the peptides can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison maybe used to design compounds expected to possess desired activities.

The invention features a method using a PSF Polypeptide, to design small molecule mimetics, agonists, or antagonists comprising determining the three dimensional structure of a PSF Polypeptide and providing a small molecule or peptide capable of binding to the PSF Polypeptide. Those skilled in the art will be able to produce small molecules or peptides that mimic the effect of the PSF Polypeptide and that are capable of easily entering the cell. Once a molecule is identified, the molecule can be assayed for its ability to bind a PSF Polypeptide, and the strength of the interaction may be optimized by making amino acid deletions, additions, or substitutions or by adding, deleting, or substituting a functional group. The additions, deletions, or modifications can be made at random or may be based on knowledge of the size, shape, and three-dimensional structure of the PSF Polypeptide.

Computer modeling techniques known in the art may also be used to observe the interaction of a PSF Polypeptide or peptide mimetic of the invention, and truncations and analogs thereof with an interacting molecule e.g. PR Polypeptide (for example, Homology Insight 11 and Discovery available from BioSym/Molecular Simulations, San Diego, Calif., U.S.A.). If computer modelling indicates a strong interaction, a PSF Polypeptide or peptide mimetic can be synthesized and tested for its ability to interfere with the binding of a motif, peptide, or mimetic with an interacting molecule.

Substances, agents, compounds, agonists and antagonists identified using a method of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The substance may be an endogenous physiological compound or it may be a natural or synthetic compound. The substance may be a PSF complex, in particular a PSF-PR Complex, which competitively inhibits the binding of PSF to its natural receptor.

The invention contemplates isolated PSF Complexes and their use in modulating a steroid receptor, a process mediated by a steroid receptor, degradation of a steroid receptor, a steroid receptor signal transduction pathway, steroid receptor transactivation, and/or a condition mediated by a steroid receptor.

In an aspect, the invention contemplates isolated PSF-PR Complexes and their use in modulating a PR Polypeptide, a process mediated by a PR Polypeptide, degradation of a PR Polypeptide, a steroid receptor signal transduction pathway, steroid receptor transactivation, and/or a condition mediated by a PR Polypeptide. In particular, the invention contemplates their use in regulating the onset of labor. In particular, the invention contemplates their use in regulating the onset of labor.

It will be understood that agonists and antagonists identified or screened using a method of the invention may act on one or more of the binding sites on interacting molecules in a PSF Complex, including agonist binding sites, competitive antagonist binding sites, non-competitive antagonist binding sites, or allosteric sites.

The substances, agents or compounds may be peptides derived from the binding sites, motifs, or domains of a PSF Polypeptide (e.g. RRMII domain), or a steroid receptor, in particular a PR Polypeptide (e.g. DBD domain and/or AF3 domain), or a PSF Complex. A peptide derived from a specific binding site, motif, or domain may encompass the amino acid sequence of a naturally occurring binding site, any portion of that binding site, motif, or domain, or other molecular entity that functions to bind or interact with an associated molecule or motif. A peptide derived from such a binding site, motif or domain will interact directly or indirectly with an associated molecule in such a way as to mimic the native binding domain. Such peptides may include competitive inhibitors, enhancers, peptide mimetics, and the like. All of these peptides as well as molecules substantially homologous, complementary or otherwise functionally or structurally equivalent to these peptides may be used for purposes of the present invention, in particular they may serve as agonists or antagonists.

Peptides may be synthesized by conventional techniques. For example, the peptides may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, supra, Vol 1, for classical solution synthesis.)

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

A substance, agent or compound that modulates or regulates a steroid receptor, in particular a PR Polypeptide; a process mediated by a steroid receptor, in particular a PR Polypeptide; degradation of a steroid receptor, in particular a PR Polypeptide; a steroid receptor signaling pathway, in particular a progesterone receptor signaling pathway; steroid receptor transactivation, in particular PR transactivation; and/or a condition mediated by a steroid receptor, in particular a PR Polypeptide, including without limitation the onset of labor, may be a molecule which interferes with the transcription and/or translation of a PSF Polynucleotide. For example, a PSF Polynucleotide may be inverted relative to its normal presentation for transcription to produce an antisense nucleic acid molecule. An antisense nucleic acid molecule may be constructed using chemical synthesis and enzymatic ligation reactions using procedures known in the art.

A substance, agent or compound that modulates a PR Polypeptide; a process mediated by a steroid receptor, in particular a PR Polypeptide; degradation of a steroid receptor, in particular a PR Polypeptide; a steroid receptor signaling pathway, in particular a progesterone receptor signaling pathway; steroid receptor transactivation, in particular PR transactivation; and/or a condition mediated by a steroid receptor, in particular a PR Polypeptide, including without limitation the onset of labor may be an aptamer.

A substance, agent, or compound may be tested in vivo and in vitro assays to ascertain if the agent, substance or compound modulates a PSF Polypeptide. The utility of a selected inhibitor or stimulator may be confirmed in cellular assays or experimental model systems.

A substance, agent or compound (e.g. motifs, peptides comprising the motifs, and peptide mimetics thereof) may be used to modulate steroid receptors, in particular PR Polypeptides, and they may be used to modulate cellular processes (such as transcription) in which the agents are introduced. The agents maybe used in a method of the invention to modulate a process mediated by a steroid receptor, in particular a PR Polypeptide; degradation of a steroid receptor, in particular a PR Polypeptide; a steroid receptor signaling pathway, in particular a progesterone receptor signaling pathway; and/or steroid receptor transactivation, in particular PR transactivation. Thus, the agents may be used in the treatment or prevention of a condition mediated by a steroid receptor, in particular a progesterone receptor.

Therefore, the present invention provides a method of conducting a drug discovery business comprising:
(a) providing one or more methods or assay systems for identifying agents by their ability to modulate a steroid receptor, in particular a PR Polypeptide; degradation of a steroid receptor, in particular a PR Polypeptide; a steroid receptor signaling pathway, in particular a progesterone receptor signaling pathway; steroid receptor transactivation, in particular PR transactivation; and/or a condition mediated by a steroid receptor, in particular a PR Polypeptide; and/or inhibit or potentiate the interaction of a PSF Polypeptide and steroir receptor in particular a PR Polypeptide;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:
(a) providing one or more assay systems for identifying agents by their ability to modulate a steroid receptor, in particular a PR Polypeptide; degradation of a steroid receptor, in particular a PR Polypeptide; a steroid receptor signaling pathway, in particular a progesterone receptor signaling pathway; steroid receptor transactivation, in particular PR transactivation; and/or a condition mediated by a steroid receptor, in particular a PR Polypeptide; and/or inhibit or potentiate the interaction of a PSF Polypeptide and steroir receptor in particular a PR Polypeptide;
(b) (optionally) conducting therapeutic profiling of agents identified in step (a) for efficacy and toxicity in animals; and
(c) licensing, to a third party, the rights for further drug development and/or sales for agents identified in step (a), or analogs thereof.

The method may further comprise the steps of preparing a quantity of the agent and/or preparing a pharmaceutical composition comprising the agent.

Diagnostic Methods

A variety of methods can be employed for the detection, diagnosis, monitoring, and prognosis of conditions described herein, or status of conditions described herein involving a PSF Polypeptide, a PSF Complex, and/or a PSF Polynucleotide, and for the identification of subjects with a predisposition to such conditions. Such methods may, for example, utilize PSF Polynucleotides, and fragments thereof, and Binding Agents (e.g. antibodies) against one or more PSF Polypeptides, including peptide fragments. In particular, the polynucleotides and antibodies may be used, for example, for (1) the detection of the presence of PSF Polynucleotide mutations, or the detection of either an over- or under-expression of PSF Polynucleotide mRNA relative to a normal state, or the qualitative or quantitative detection of alternatively spliced forms of PSF Polynucleotide transcripts which may correlate with certain conditions or susceptibility toward a condition; and (2) the detection of either an over- or an under-abundance of one or more PSF Polypeptides relative to a normal state or a different stage of a condition, or the presence of a modified (e.g. less than full length) PSF Polypeptide which correlates with a condition or state, or a progression toward a condition, or a particular type or stage of a condition.

The methods described herein can be adapted for diagnosing and monitoring a condition mediated by a progesterone receptor, in particular labor or pre-term labor, by detecting one or more PSF Polypeptides, PSF Complex, or PSF Polynucleotides in biological samples from a subject. These applications require that the amount of PSF Polypeptides, PSF Complexes, or PSF Polynucleotides quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different stages or types of condition, may be established by prospective and/or retrospective statistical studies. Healthy subjects who have no clinical evidence of a condition or abnormalities may be selected for statistical studies. Diagnosis may be made by a finding of statistically different levels of detected PSF Polypeptides, PSF Complexes, or PSF Polynucleotides associated with a condition such as pre-term labor, compared to a control sample or previous levels quantitated for the same subject.

In an aspect of the invention, a method is provided for diagnosing or monitoring in a subject a condition mediated by a PR Polypeptide, in particular a condition requiring regulation of labor, comprising detecting a PSF Polypeptide, PSF Complex, and/or PSF Polynucleotide in a sample from the subject. In an embodiment of a diagnostic method of the invention, a method is provided for diagnosing increased risk of pre-term labor in a subject comprising detecting a PSF Polypeptide or PSF Polynucleotide in a sample from the subject.

The methods described herein may be used to predict or evaluate the probability of pre-term labor or onset of true labor or pre-term labor, for example, in a sample freshly removed from a host. Such methods can be used to detect labor or pre-term labor and help in the diagnosis and prognosis of labor or pre-term labor. The methods can be used to detect the potential for labor or pre-term labor and to monitor labor or pre-term labor or a therapy.

The invention also contemplates a method for detecting pre-term labor, or onset of labor or pre-term labor, comprising producing a profile of levels of one or more PSF Polypeptides and/or PSF Polynucleotides, and other markers associated with labor or pre-term labor in a sample (e.g. cells) from a patient, and comparing the profile with a reference to identify a profile for the patient indicative of labor or pre-term labor.

The invention contemplates a method for determining the likelihood of occurrence of pre-term labor in a pregnant mammal comprising detecting a PSF Polypeptide, PSF Complex, and/or PSF Polynucleotide in a sample from the subject.

The invention also contemplates a method for distinguishing pre-term (false) labor and true labor in a pregnant mammal comprising detecting a PSF Polypeptide, PSF Complex, and/or PSF Polynucleotide in a sample from the subject.

The invention provides a method for assaying whether a pregnant mammal is in imminent delivery of its fetus in pre-term labor comprising contacting a maternal sample of the mammal with a reagent that detects a PSF Polypeptide, PSF Complex, and/or PSF Polynucleotde, and measuring the level of PSF Polypeptide, PSF Complex, and/or PSF Polynucleotide in the sample.

The methods described herein may also use multiple markers for a condition described herein, in particular labor or pre-term labor. Therefore, the invention contemplates a method for analyzing a biological sample for the presence of one or more PSF Polypeptides, PSF Complexes, and PSF Polynucleotides, and other markers that are specific indicators of the condition. The methods described herein may be modified by including reagents to detect the additional markers.

PSF Polypeptide Diagnostic Methods

A PSF Polypeptide or complex thereof (e.g. PSF Complex), may be detected in a variety of samples from a patient. Examples of suitable samples include cells (e.g. fetal or maternal); and, fluids (fetal or maternal), including for example, serum, plasma, amniotic fluid, vaginal fluid, saliva, and conditioned medium from fetal or maternal cells.

A PSF Polypeptide or complex thereof may be detected using a binding agent. Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988).

Binding agents may be used as diagnostic or prognostic reagents and they may be used to detect abnormalities in the level of expression of a PSF Polypeptide, or abnormalities in the structure, and/or temporal, tissue, cellular, or subcellular location of a PSF Polypeptide. They may also be used to screen potentially therapeutic compounds in vitro to determine their effects on conditions involving one or more PSF Polypeptides, and other conditions described herein, or to assess or monitor the efficacy of particular therapies.

In general, the presence or absence of a PSF Polypeptide or PSF Complex in a subject may be determined by (a) contacting a sample from the subject with a binding agent that interacts with a PSF Polypeptide and/or PSF Complex; (b) detecting in the sample a level of polypeptide or complex that binds to the binding agent; and (c) comparing the level of polypeptide with a predetermined standard or cut-off value.

In the context of certain methods of the invention, a sample, binding agents (e.g. antibodies specific for one or more PSF Polypeptides), PSF Polypeptides, or PSF Complexes may be immobilized on a carrier or support as described herein. Immobilization typically entails separating the binding agent from any free analytes (e.g. free PSF Polypeptide or free PSF Complex) in the reaction mixture.

A binding agent can directly or indirectly interact with a PSF Polypeptide or PSF Complex.

Binding agents may be labeled using conventional methods with a detectable substance. Binding agents, including antibodies to a PSF Polypeptide or PSF Complex, or peptides that interact with a PSF Polypeptide or PSF Complex, may also be indirectly labeled with a ligand binding partner. For example, the antibodies, or peptides may be conjugated to one partner of a ligand binding pair, and the PSF Polypeptide may be coupled to the other partner of the ligand binding pair. Representative examples include avidin-biotin, and riboflavin-riboflavin binding protein. In an embodiment the binding agent (e.g. antibodies) are biotinylated. Methods for conjugating binding agents such as antibodies with a ligand binding partner may be readily accomplished by one of ordinary skill in the art (see Wilchek and Bayer, “The Avidin-Biotin Complex in Bioanalytical Applications,” Anal. Biochem. 171:1-32, 1988).

Indirect methods may also be employed in which a primary binding agent-binding partner interaction is amplified by introducing a second agent. In particular, a primary PSF Polylpeptide-antibody reaction may be amplified by the introduction of a second antibody, having specificity for the antibody reactive against PSF Polypeptides. By way of example, if the antibody having specificity against PSF Polypeptides is a rabbit IgG antibody, the second antibody may be goat anti-rabbit gamma-globulin labeled with a detectable substance as described herein.

The presence of a PSF Polypeptide may be determined by measuring the binding of the PSF Polypeptide to molecules (or parts thereof) which are known to interact with a PSF Polypeptide including but not limited to a PR Polypeptide. In aspects of the invention, peptides derived from sites on the progesterone receptor which bind to a PSF Polypeptide may be used (e.g. AF3 and DNA binding domain). A peptide derived from a specific site on the receptor may encompass the amino acid sequence of a naturally occurring binding site, any portion of that binding site, or other molecular entity that functions to bind an associated molecule. A peptide derived from such a site will interact directly or indirectly with an associated molecule in such a way as to mimic the native binding site. Such peptides may include competitive inhibitors, enhancers, peptide mimetics, and the like as discussed herein.

In other aspects of the invention, the binding agent is an antibody. Antibodies specifically reactive with one or more PSF Polypeptides or PSF Complexes, or derivatives, such as enzyme conjugates or labeled derivatives, may be used to detect one or more PSF Polypeptide or PSF Complex in various samples (e.g. biological materials).

In particular the invention provides a diagnostic method for monitoring or diagnosing a condition mediated by a progesterone receptor, in particular pre-term labor, in a subject by quantitating PSF Polypeptides or PSF Complexes in a biological sample from the subject comprising reacting the sample with antibodies specific for PSF Polypeptides, which are directly or indirectly labeled with detectable substances and detecting the detectable substances. In a particular embodiment of the invention, PSF Polypeptides are quantitated or measured.

In an aspect of the invention, a method for detecting a condition mediated by a progesterone receptor, in particular pre-term labor, is provided comprising:
(a) obtaining a sample suspected of containing PSF Polypeptides or PSF Complexes associated with pre-term labor;
(b) contacting the sample with antibodies that specifically bind to the PSF Polypeptides or PSF Complexes under conditions effective to bind the antibodies and form complexes;
(c) measuring the amount of PSF Polypeptides or PSF Complexes present in the sample by quantitating the amount of the antibody-PSF Polypeptide or antibody-PSF Complex complexes; and
(d) comparing the amount of PSF Polypeptides or PSF Complexes present in the samples with the amount of PSF Polypeptides or PSF Complexes in a control, wherein a change or significant difference in the amount of PSF Polypeptides or PSF Complexes in the sample compared with the amount in the control is indicative of the condition, in particular pre-term labor.

The amount of antibody complexes may also be compared to a value representative of the amount of antibody complexes from an individual not at risk of, or afflicted with, a condition or having a condition at different stages. A significant difference in antibody complex formation may be indicative of an advanced condition, or an unfavourable prognosis.

In embodiments of the methods of the invention, PSF Polypeptides or PSF Complexes are detected in samples and higher levels, in particular significantly higher levels compared to a control (normal or benign) is indicative of onset or initiation of labor.

In an embodiment, the invention contemplates a method for monitoring the progression of a condition mediated by a progesterone receptor, in particular pre-term labor, in an individual, comprising:
(a) contacting antibodies which bind to PSF Polypeptides or PSF Complexes with a sample from the individual so as to form complexes comprising the antibodies and PSF Polypeptides or PSF Complexes in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of a condition, condition stage, and/or progression of the condition in the individual.

In methods of the invention the step of contacting a sample with a binding agent (e.g. antibodies) may be accomplished by any suitable technique so that detection can occur. In particular, antibodies may be used in any known immunoassays that rely on the binding interaction between antigenic determinants of one or more PSF Polypeptide or PSF Complex and the antibodies. Immunoassay procedures for in vitro detection of antigens in fluid samples are well known in the art, as well as widely establilshed and used in the commercial diagnostic industry. [See for example, Paterson et al., Int. J. Can. 37:659 (1986) and Burchell et al., Int. J. Can. 34:763 (1984) for a general description of immunoassay procedures]. Qualitative and/or quantitative determinations of PSF Polypeptides or PSF Complexes in a sample may be accomplished by competitive or non-competitive immunoassay procedures in either a direct or indirect format. Detection of PSF Polypeptides or PSF Complexes using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

Thus, the present invention provides means for determining one or more PSF Polypeptides in a sample by measuring one or more PSF Polypeptides by immunoassay. According to an embodiment of the invention, an immunoassay for detecting PSF Polypeptides in a biological sample comprises contacting antibodies that specifically bind to PSF Polypeptides or PSF Complexes in the sample under conditions that allow the formation of first complexes comprising antibodies and PSF Polypeptides or PSF Complexes and determining the presence or amount of the first complexes as a measure of the amount of PSF Polypeptides or PSF Complexes contained in the sample.

Antibodies may be used to detect and quantify one or more PSF Polypeptides in a sample in order to diagnose and treat pathological states. In particular, the antibodies may be used in immunohistochemical analyses, for example, at the cellular and sub-subcellular level, to detect one or more PSF Polypeptides, to localize them to particular cells and tissues and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a condition described herein is contacted with antibodies, preferably monoclonal antibodies recognizing PSF Polypeptides. The site at which the antibodies are bound is determined by selective staining of the sample by standard immunohistochemical procedures. The tissue sample may be normal tissue or abnormal/disease tissue.

Antibodies specific for one or more PSF Polypeptide may be labelled with a detectable substance and localised in tissues and cells based upon the presence of the detectable substance. Cytochemical techniques known in the art for localizing antigens using light and electron microscopy may be used to detect PSF Polypeptides or PSF Complexes.

It will be evident to a skilled artisan that a variety of immunoassay methods can be used to measure one or more PSF Polypeptides. In general, an immunoassay method may be competitive or noncompetitive.

In an aspect of the invention a competitive method is provided employing an immobilized or immobilizable antibody to a PSF Polypeptide and a labeled form of a PSF Polypeptide. Sample PSF Polypeptides and labeled PSF Polypeptides compete for binding to antibodies to PSF Polypeptides. After separation of the resulting labeled PSF Polypeptides that have become bound to antibodies (bound fraction) from that which has remained unbound (unbound fraction), the amount of the label in either bound or unbound fraction is measured and may be correlated with the amount of PSF Polypeptides in the test sample in any conventional manner, e.g., by comparison to a standard curve.

In another aspect, a non-competitive method is used for the determination of PSF Polypeptides, with the most common method being the “sandwich” method. In this assay, two antibodies to PSF Polypeptides are employed. One of the antibodies to PSF Polypeptides is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises PSF Polypeptides bound by (“sandwiched” between) the capture and detection antibodies. In an embodiment, the label may be measured without separating the capture antibodies and liquid test mixture.

The above-described immunoassay methods and formats are intended to be exemplary and are not limiting. Other methods now or hereafter developed for the determination of a PSF Polypeptide or PSF Complex are included within the scope hereof.

PSF Polynucleotide Diagnostic Methods

A condition mediated by a steroid receptor, in particular a condition mediated by a progesterone receptor, more particularly pre-term labor, or stage or type of same, may be detected based on the level of PSF Polynucleotides in a sample. Techniques for detecting polynucleotides such as polymerase chain reaction (PCR) and hybridization assays are well known in the art.

Probes may be used in hybridization techniques to detect polynucleotide markers. The technique generally involves contacting and incubating polynucleotides (e.g. recombinant DNA molecules, cloned genes) obtained from a sample from a patient or other cellular source with a probe under conditions favourable for the specific annealing of the probes to complementary sequences in the polynucleotides. After incubation, the non-annealed nucleic acids are removed, and the presence of polynucleotides that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a PSF Polynucleotide, preferably they comprise 10-30, 10-40, 15-40, 20-50, 40-80, 50-150, or 80-120 nucleotides.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, supra. The nucleic acid probes may be used to detect PSF Polynucleotides in human samples, e.g. serum or plasma. In aspects of the invention the nucleotide probes are useful in the diagnosis, prediction, management and control of pre-term labor or labor involving one or more PSF Polynucleotides, in monitoring the progression of pre-term labor or labor; or monitoring a therapeutic treatment.

The levels of mRNA or polynucleotides derived therefrom can be determined using hybridization methods known in the art. For example, RNA can be isolated from a sample and separated on a gel. The separated RNA can then be transferred to a solid support and nucleic acid probes representing one or more markers can be hybridized to the solid support and the amount of marker-derived RNA can be determined. Such determination can be visual, or machine-aided (e.g. use of a densitometer). Dot-blot or slot-blot may also be used to determine RNA. RNA or nucleic acids derived therefrom from a sample are labeled, and then hybridized to a solid support containing oligonucleotides derived from one or more marker genes that are placed on the solid support at discrete, easily-identifiable locations. Hybridization, or the lack thereof, of the labeled RNA to the solid support oligonucleotides is determined visually or by densitometer.

The detection of PSF Polynucleotides may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a PSF Polynucleotide(s) derived from a sample, wherein at least one of the oligonucleotide primers is specific for (i.e. hybridizes to) a PSF Polynucleotide. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a PSF Polynucleotide; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of PSF Polynucleotide expression. For example, RNA may be isolated from a cell type or tissue known to express a PSF Polynucleotide and tested utilizing the hybridization (e.g. standard Northern analyses) or PCR techniques referred to herein. The primers and probes may be used in the above-described methods in situ i.e. directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a PSF Polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected condition described herein (e.g. suspected pre-term labor) and an individual who is not predisposed to such condition. The reaction may be performed on several dilutions of cDNA spanning at least two orders of magnitude. A significant difference in expression in several dilutions of the subject sample as compared to the same dilutions of the normal sample may be considered positive for the presence of the condition (e.g. pre-term labor).

In an aspect, the invention provides methods for determining the presence or absence of a condition described herein, in particular pre-term labor, in a subject comprising (a) contacting a sample obtained from the subject with oligonucleotides that hybridize to PSF Polynucleotides; and (b) detecting in the sample levels of polynucleotides that hybridize to the PSF Polynucleotides relative to a predetermined cut-off value, and therefrom determining the presence or absence of pre-term labor in the subject.

In another aspect, the invention provides a method wherein PSF Polynucleotides that are mRNA are detected by (a) isolating mRNA from a sample and combining the mRNA with reagents to convert it to cDNA; (b) treating the converted cDNA with amplification reaction reagents and nucleic acid primers that hybridize to a PSF Polynucleotide, to produce amplification products; (d) analyzing the amplification products to detect an amount of PSF Polynucleotide mRNA; and (e) comparing the amount of mRNA to an amount detected against a panel of expected values for normal subjects derived using similar nucleic acid primers.

PSF Polynucleotide-positive samples or alternatively higher levels in patients compared to a control (e.g. normal sample) may be indicative of a condition, in particular pre-term labor or advanced pre-term labor, and/or that the patient is not responsive to or tolerant of a therapy. Alternatively, negative samples or lower levels compared to a control (e.g. normal samples or negative samples) may also be indicative of a condition, and/or that a patient is not responsive to or tolerant of a therapy.

In particular aspects, the methods are used to determine the presence or absence of pre-term labor, determine the likelihood of occurrence of pre-tem labor in a subject, or distinguish pre-term labor from true labor. In a method for diagnosing or identifying onset of labor and particularly pre-term labor, higher levels of the markers, in particular significantly higher levels of PSF Polynucleotides in patients compared to a control (e.g. normal) are indicative of pre-term labor or onset of labor, or the likelihood of occurrence of pre-term labor.

Oligonucleotides or longer fragments derived from PSF Polynucleotides may be used as targets in a micro-array. The micro-array can be used to simultaneously monitor the expression levels of PSF genes. The micro-array can also be used to identify genetic variants, mutations, and polymorphisms. The information from the micro-array may be used to determine gene function, to understand the genetic basis of a condition (e.g. pre-term labor), to diagnose a condition (e.g. pre-term labor), and to develop and monitor the activities of therapeutic agents. Thus, the invention also includes an array comprising one or more PSF Polynucleotides, and optionally other markers. The array can be used to assay expression of PSF Polynucleotides in the array. The invention allows the quantitation of expression of one or more PSF Polynucleotides. Arrays are also useful for ascertaining differential expression patterns of PSF Polynucleotides as described herein, and optionally other markers, in normal and abnormal samples. This may provide a battery of nucleic acids that could serve as molecular targets for diagnosis or therapeutic intervention.

Kits

The invention also relates to kits for carrying out the methods of the invention. The kits comprise instructions, negative and positive controls, and means for direct or indirect measurement of PSF Polypeptides, PSF Complexes, or PSF Polynucleotides. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific PSF Polypeptide, PSF Polynucleotide, or binding agent (e.g. antibody) described herein, which may be conveniently used, e.g., in clinical settings to screen and diagnose patients and to screen and identify those individuals exhibiting a predisposition to developing a condition mediated by a steroid receptor, in particular a condition mediated by a progesterone receptor, more particularly pre-term labor.

In an embodiment, a container with a kit comprises a binding agent as described herein. By way of example, the kit may contain antibodies or antibody fragments which bind specifically to epitopes of PSF Polypeptides, and optionally other markers, antibodies against the antibodies labelled with an enzyme, and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more PSF polypeptide comprising a sequence of SEQ ID NOs. 1, 2, 3, 4, 5, and 21, and means for detecting binding of the antibodies to their epitope associated with a condition mediated by a progesterone receptor, in particular pre-term labor, either as concentrates (including lyophilized compositions), which may be further diluted prior to use or at the concentration of use, where the vials may include one or more dosages.

A kit may be designed to detect the level of polynucleotides encoding one or more PSF Polynucleotides in a sample. In an embodiment, the polynucleotides encode one or more polynucleotides comprising a sequence of SEQ ID Nos. 7, 8, or 9. Such kits generally comprise at least one oligonucleotide probe or primer, as described herein, that hybridizes to a PSF Polynucleotide. Such an oligonucleotide may be used, for example, within a PCR or hybridization procedure.

The invention provides a kit containing a micoarray described herein ready for hybridization to target PSF Polynucleotides, plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

In an aspect, the invention provides a kit comprising a reagent that detects a PSF Polypeptide, PSF Polynucleotide, or PSF-PR Complex, and instructions or package insert or label for assaying whether a pregnant mammal is in imminent delivery of its fetus in preterm labor. The kit may further comprise a detection means and/or microtiter plates, a PSF Polypeptide, PSF Complex or PSF Polynucleotide standard or tracer, which is typically labeled, and an immobilized reagent that detects PSF Polypeptide, PSF Complex, or PSF Polynucleotide, which is used to capture the PSF Polypeptide, PSF Complex, or PSF Polynucleotide.

The invention contemplates a kit for assessing the presence of cells and tissues associated with a condition mediated by a progesterone receptor, in particular pre-term labor or onset of pre-term labor, wherein the kit comprises antibodies specific for one or more PSF Polypeptides or PSF Complexes, or primers or probes for PSF Polynucleotides, and optionally probes, primers or antibodies specific for other markers associated with the condition (e.g. fibronectin associated with pre-term labor).

The reagents suitable for applying the screening methods of the invention to evaluate compounds may be packaged into convenient kits described herein providing the necessary materials packaged into suitable containers.

The invention relates to a kit for assessing the suitability of each of a plurality of test compounds for inhibiting a condition mediated by a progesterone receptor in particular pre-term labor or onset of pre-term labor in a patient. The kit comprises reagents for assessing one or more PSF Polypeptides, PSF Complexes, or PSF Polynucleotides, and optionally a plurality of test agents or compounds.

Additionally the invention provides a lit for assessing the potential of a test compound to contribute to a condition mediated by a steroid receptor, in particular a condition mediated by a progesterone receptor, more particularly pre-term labor. The kit comprises cells and tissues associated with the condition and reagents for assessing one or more PSF Polypeptides, PSF Complexes, PSF Polynucleotides, and optionally other markers associated with the condition.

Applications

The invention relates to methods of modulating a PSF Polypeptide, a steroid receptor, a PSF Complex, a process mediated by a steroid receptor, a steroid receptor signal transduction pathway in a cell, degradation of a steroid receptor, steroid receptor transactivation, and/or modulating a condition mediated by a steroid receptor, and/or inhibiting or potentiating the interaction of a steroid receptor and a PSF Polypeptide, in a subject comprising administering an effective amount of a PSF Polypeptide, a PSF Polynucleotide, PSF Complex, or an agonist or antagonist thereof In an aspect, the invention relates to methods of modulating a PSF Polypeptide, a PSF Complex comprising an androgen receptor, a process mediated by an androgen receptor, an androgen receptor signal transduction pathway in a cell involving a PSF Polypeptide and an androgen receptor, degradation of an androgen receptor, androgen receptor transactivation, and/or modulating a condition mediated by an androgen receptor, and/or inhibiting or potentiating the interaction of an androgen receptor and a PSF Polypeptide in a subject comprising administering an effective amount of a PSF Polypeptide, a PSF Polynucleotide, PSF Complex, or an agonist or antagonist thereof. In some aspects of the invention for treating or preventing cancer, in particular a cancer discussed herein, the method comprises administering an effective amount of a PSF Polypeptide, a PSF Polynucleotide, PSF Complex, or an agonist thereof.

In another aspect, the invention relates to methods of modulating a PSF Polypeptide, a PSF-PR Complex, a process mediated by a PR Polypeptide, a progesterone receptor signal transduction pathway in a cell involving a PSF Polypeptide and a PR Polypeptide, degradation of a PR Polypeptide, progesterone receptor transactivation, and/or modulating a condition mediated by a progesterone receptor, and/or inhibiting or potentiating the interaction of a PR Polypeptide and a PSF Polypeptide in a subject comprising administering an effective amount of a PSF Polypeptide, a PSF Polynucleotide, PSF Complex, or an agonist or antagonist thereof.

Therefore, the invention provides methods for regulating, controlling, managing, inhibiting, treating or preventing a condition mediated by a steroid receptor, in particular a condition mediated by a progesterone receptor comprising directly or indirectly modulating (e.g. inhibiting or stimulating) a PSF Polypeptide, PSF Polynucleotide and/or PSF Complex.

In particular applications of the invention, methods are provided for regulating, controlling, managing, and/or inhibiting the onset of labor comprising directly or indirectly modulating (e.g. inhibiting or stimulating) a PSF Polypeptide, PSF Polynucleotide and/or PSF Complex.

The invention contemplates methods for controlling and managing spontaneous or surgically induced pre-term labor. In an aspect a method is provided for controlling and managing pre-term labor by administering to a pregnant female an antagonist of a PSF Polypeptide, PSF Polynucleotide or PSF Complex, in order to prevent or inhibit and control preterm labor. Another aspect is a method for control, management and inhibition of preterm labor by manipulating levels of a PSF Polypeptide, PSF Polynucleotide or PSF Complex. Still another aspect of the invention is a method for controlling and managing pre-term labor by administering to a pregnant female compounds which alter a PSF Polypeptide, a PSF Polynucleotide and/or a PSF Complex.

In an aspect of the invention, a method is provided for control, management, and inhibition of onset of labor in a subject comprising administering an effective amount of a substance which is an antagonist or inhibitor of a PSF Polypeptide. In particular, methods are provided for treating a women suffering from or who may be susceptible to pre-term labor comprising administering a safe and non-toxic concentration of an antagonist of a PSF Polypeptide, PSF Complex, and/or PSF Polynucleotide during the preterm labor.

The invention also provides a method for avoiding pre-term or premature labor in a pregnant mammal comprising administering to said mammal during labor, but before an infant is to be delivered, an effective amount of an antagonist of a PSF Polypeptide, a PSF Complex, and/or a PSF Polynucleotide.

In a further aspect, the invention provides a method for avoiding premature labor in a pregnant mammal comprising contacting a maternal serum sample or amniotic fluid sample of the mammal with a reagent that detects a PSF Polypeptide, PSF Complex, and/or PSF Polynucleotide; measuring the level of PSF Polypeptide, PSF Complex, and/or PSF Polynucleotide in the serum or amniotic fluid; and if the measurement of PSF Polypeptide, PSF Complex, and/or PSF Polynucleotide levels indicates that preterm labor is imminent or is occurring, administering to the mammal during labor, but before an infant is to be delivered, an effective amount of an antagonist of a PSF Polypeptide, PSF Complex, and/or PSF Polynucleotide to avoid premature labor in the mammal.

An aspect of the invention is a method for for inducing labor in over term pregnancy. Thus, the invention provides a method for inducing labor in a subject comprising administering an effective amount of a PSF Polypeptide, PSF Polynucleotide, or PSF Complex.

In an embodiment of the invention a method is provided for treating a woman suffering from, or who may be susceptible to pre-term labor comprising administering therapeutically effective dosages of an inhibitor of PSF, or a substance identified in accordance with the methods of the invention. Treatment with the inhibitor may commence prior to or after onset of labor, and may continue until measured PSF levels are within the normal range. For the purposes of the present invention normal PSF levels are defined as those levels typical for pregnant women who do not suffer from pre-term labor. Treatment with the inhibitor is discontinued after PSF levels are within normal range, and before any adverse effects of administration of the inhibitor are observed. Inhibition may be reversed for example by treatment with a proteosomal inhibitor.

A PSF Polypeptide, PSF Complex, or PSF Polynucleotide, or agonists thereof, and agents, substances and compounds identified using a method of the invention, have particular application in contraception, and they may be administered alone, in combination with a PR Polypeptide agonist, or in combination or sequentially with a partial ER antagonist such as tamoxifen or an estrogen agonist (e.g. ethinyl estradial). In addition, a PSF Polypeptide, PSF Complex, or PSF Polynucleotides or agonists thereof, and agents, substances and compounds identified using a method of the invention, may have particular application in the treatment of hormone dependent breast cancers, uterine and ovarian cancers, and hormone dependent prostate cancer in men. They may also be useful for the treatment of non-malignant chronic conditions such as fibroids, endometriosis, and hormone replacement therapy for post menopausal patients in combination with a partial ER antagonist such as tamoxifen. Additional uses of a PSF Polypeptide, PSF Complex, or PSF Polynucleotide or agonists thereof, and agents, substances and compounds identified using a method of the invention, include the synchronization of the estrus in domestic animals.

PSF Polypeptides, PSF Polynucleotides, PSF Complexes, and agonists and antagonists thereof (e.g. binding agents), and agents, compounds, and substances identified using a method of the invention may be formulated into compositions for administration to subjects. Therefore the present invention also relates to a pharmaceutical composition comprising an effective amount of a PSF Polypeptide, PSF Complex, and/or PSF Polynucleotide, or an agonist or antagonist thereof, or an agent, compound or substance identified using a method of the invention. The pharmaceutical compositions can be used in the methods of the invention.

In particular a pharmaceutical composition of the invention can be adapted for administration to a subject for the prevention or treatment of a condition mediated by a progesterone receptor, in particular for prevention or treatment of pre-term labor, or induction of labor. Therefore, one or more inhibitors (i.e. antagonists) or one or more stimulators (i.e. agonists) of PSF, or substances selected in accordance with the methods of the invention including binding agents, may be incorporated into a composition adapted for regulating the onset of labor. In an embodiment of the invention, a composition is provided for treating a woman suffering from, or who may be susceptible to pre-term labor, comprising a therapeutically effective amount of an antagonist or inhibitor of a PSF Polypeptide, or substance, agent or compound selected in accordance with the methods of the invention, and a carrier, diluent, or excipient.

An aspect of the invention provides pharmaceutical compositions comprising agents which produce, control or alter PSF Polypeptide, PSF Complex or PSF Polynucleotide availability or levels, which compositions are useful for control of pre-term labor or for induction of labor in over term pregnancy and which compositions are administered to a pregnant woman.

A composition of the invention can be intended for administration to subjects such as humans or animals, and will be formulated, dosed and administered in a fashion consistent with good medical or veterinary practices.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

Compositions of the present invention can be administered by any means that produce contact of the active agent(s) with the agent's sites of action in the body of a subject or patient to produce the desired therapeutic or preventive effects. Suitable means of administration include oral, intranasal, inhalation, intraperitoneal, subcutaneous, intramuscular, transdermal, sublingual, intrapulmonary, intraarterial, or intravenous administration. The active ingredients can be administered simultaneously or sequentially and in any order at different points in time, to provide the desired effects. A composition of the invention can be formulated for sustained release, for delivery locally or systemically. It lies within the capability of a skilled physician or veterinarian to select a form and route of administration that optimizes the effects of the compositions and treatments of the present invention to provide desired therapeutic or preventive effects.

The methods of the invention for use on subjects/individuals/patients contemplate prophylactic as well as therapeutic or curative use. Typical subjects for treatment include persons susceptible to, suffering from or that have suffered a condition mediated by a steroid receptor, in particular a condition mediated by a progesterone receptor.

An “effective amount” or “therapeutically effective amount” of an active ingredient (e.g. a PSF Polypeptide antagonist or inhibitor) or composition of the invention is an amount effective to elicit the desired therapeutic or preventive response but insufficient to cause a toxic reaction. The dosage for the compositions is determined by the attending physician or veterinarian taking into account factors such as the condition, body weight, diet of the subject, and the time of administration. In an aspect of the invention for treating or preventing pre-term labor, the effective amount is the minimum amount necessary to prevent premature delivery of an infant. Such amount is preferably below the amount that is toxic to the patient or renders the patient significantly more susceptible to infections.

In some aspects of the invention, an effective amount or therapeutically effective amount or dosage refers to an amount of an antagonist or inhibitor of a PSF Polypeptide, PSF-PR Complex, and/or PSF Polynucleotide effective to maintain steroid receptor levels or functional activity of steroid receptors. For example, a therapeutically effective amount of an antagonist, e.g. an amount sufficient to lower levels of a PSF Polypeptide to normal levels, may be about 1 to 1000 μg/kg/day, in particular 1 to 200 μg/kg/day. In methods for the treatment of pre-term labor a therapeutically effective dosage may be an amount of an antagonist or inhibitor of a PSF Polypeptide and/or PSF Polynucleotide effective to maintain progesterone receptor levels or functional activity thus inhibiting the onset of labor. A method of the invention may involve a series of administrations of the composition. Such a series may take place over a period of 7 to about 21 days and one or more series may be administered. The composition may be administered initially at the low end of the dosage range and the dose will be increased incrementally over a preselected time course.

A PSF Polypeptide, PSF Complex, PSF Polynucleotide, or agonist or antagonist including agents, substances, or compounds identified in accordance with the methods of the invention may be administered by gene therapy techniques using genetically modified cells or by directly introducing genes encoding the inhibitors or stimulators (e.g. agonists or antagonists) of a PSF Polypeptide, or substances into cells in vivo. Cells may be transformed or transfected with a recombinant vector (e.g. retroviral vectors, adenoviral vectors and DNA virus vectors). Genes encoding inhibitors or stimulators, or agents, substances or compounds, maybe introduced into cells of a subject in vivo using physical techniques such as microinjection and electroporation or chemical methods such as coprecipitation and incorporation of DNA into liposomes. Antisense molecules may also be introduced in vivo using these conventional methods.

A PSF Polypeptide or PSF Complex may be targets for immunotherapy. Immunotherapeutic methods include the use of antibody therapy. In one aspect, the invention provides PSF Polypeptide or PSF Complex antibodies that may be used to treat or prevent a condition mediated by a steroir receptor, in particular a condition mediated by a progesterone receptor, more particularly, pre-term labor. In a particular aspect, the invention provides a method of preventing, inhibiting or reducing pre-term labor or the onset of pre-term labor, comprising administering to a patient an antibody which binds specifically to a PSF Polypeptide and/or PSF Complex in an amount effective to prevent, inhibit, or reduce pre-term labor or the onset of pre-term labor.

The methods of the invention contemplate the administration of single PSF Polypeptide and/or PSF Complex antibodies as well as combinations, or “cocktails”, of different individual antibodies such as those recognizing different epitopes of other markers. Such cocktails may have certain advantages inasmuch as they contain antibodies that bind to different epitopes of a PSF Polypeptide and/or a PSF Complex and/or exploit different effector mechanisms. Such antibodies in combination may exhibit synergistic therapeutic effects. In addition, the administration of PSF Polypeptide or PSF Complex specific antibodies maybe combined with other therapeutic agents. PSF Polypeptide or PSF Complex specific antibodies maybe administered in their “naked” or unconjugated form, or may have therapeutic agents conjugated to them.

Treatment will generally involve the repeated administration of the antibody preparation via an acceptable route of administration at an effective dose. Dosages will depend upon various factors generally appreciated by those of skill in the art, including the etiology of the condition, stage of the condition, the binding affinity and half life of the antibodies used, the degree of PSF Polypeptide or PSF Complex expression in the patient, the desired steady-state antibody concentration level, frequency of treatment, and the influence of any therapeutic agents used in combination with a treatment method of the invention. A determining factor in defining the appropriate dose is the amount of a particular antibody necessary to be therapeutically effective in a particular context. Repeated administrations may be required to achieve a desired effect. Direct administration of PSF Polypeptide or PSF Complex antibodies is also possible and may have advantages in certain situations.

Patients may be evaluated for PSF Polypeptides or PSF Complexes in order to assist in the determination of the most effective dosing regimen and related factors. The assay methods described herein, or similar assays, may be used for quantitating a PSF Polypeptide or PSF Complex levels in patients prior to treatment. Such assays may also be used for monitoring throughout therapy, and may be useful to gauge therapeutic success in combination with evaluating other parameters such as levels of PSF Polypeptides or PSF Complexes.

PSF Polynucleotides associated with a condition mediated by a steroid receptor, in particular a condition mediated by a progesterone receptor, more particularly pre-term labor, can be turned off by transfecting a cell or tissue with vectors that express high levels of a desired PSF Polynucleotide. Such constructs can inundate cells with untranslatable sense or antisense sequences. Even in the absence of integration into the DNA, such vectors may continue to transcribe RNA molecules until all copies are disabled by endogenous nucleases.

Vectors derived from retroviruses, adenovirus, herpes or vaccinia viruses, or from various bacterial plasmids, may be used to deliver PSF Polynucleotides to a targeted organ, tissue, or cell population. Methods well known to those skilled in the art may be used to construct recombinant vectors that will express PSF Polynucleotides such as antisense. (See, for example, the techniques described in Sambrook et al (supra) and Ausubel et al (supra).) Methods for introducing vectors into cells or tissues include those methods discussed herein and which are suitable for in vivo, in vitro and ex vivo therapy. For example, delivery by transfection and by liposome are well known in the art.

Modifications of gene expression can be obtained by designing antisense molecules, DNA, RNA or PNA, to the regulatory regions of a PSF Polynucleotide, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polyinerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

The invention contemplates engineered hammerhead motif ribozyme molecules that can specifically and efficiently catalyze endonucleolytic cleavage of PSF Polynucleotides. Ribozymes are enzymatic RNA molecules that catalyze the specific cleavage of RNA. Ribozymes act by sequence-specific hybridization of the ribozyme molecule to complementary target RNA, followed by endonucleolytic cleavage. Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

The compositions and methods described herein are indicated as therapeutic agents or methods either alone or in conjunction with other therapeutic agents or other forms of treatment. They may be combined or formulated with one or more therapies or agents used to treat a condition described herein. Compositions of the invention may be administered concurrently, separately, or sequentially with other therapeutic agents or therapies. In a method of the invention for preventing or treating pre-term labor a composition of the invention is combined or formulated with one or more tocolytic agent or 5 alpha-reductase inhibitor (U.S. Pat. No. 5,872,126 to Cukierski et al), including without limitation beta-adrenergic agonists, magnesium sulfate, ethanol, oxytocin antagonists, calcium transport blockers, prostaglandin synthesis inhibitors, nitric oxide donors, phosphodiesterase inhibitors, and/or progestins.

The following non-limiting examples are illustrative of the present invention:

### EXAMPLE 1

Progesterone is an essential regulator of the reproductive events associated with the establishment and maintenance of pregnancy through its ligand-activated progesterone receptor (PR). Progesterone actions include the suppression of genes encoding contraction-associated proteins (CAPs, e.g. oxytocin receptor, prostaglandin receptor, connexin43) that are required for myometrial activation and the onset of labor. In the human, progesterone levels remain elevated through labour and even in species where progesterone levels fall at term, concentrations are likely sufficiently high to inhibit CAP gene expression. This suggests there must be an active mechanism for inducing a functional withdrawal of progesterone at term. The objective of this study was to identify novel PR-interacting proteins that might block the PR signaling pathway at term in human pregnancy. GST-PR fusion proteins were used to “pulldown” interacting proteins in myometrial cell homogenates and the identity of these proteins was determined by MALDI-TOP Mass Spectrometry. One of the PR-interacting proteins was identified as PSF, a previously recognized RNA splicing factor. The interaction between PR and PSF was confirmed by iii vivo (mammalian two-hybrid system) and in vitro (GST-pull down assay using purified proteins) protein interaction assays. PSF was found to interact with both the PRA and PRB isoforms. The interaction domains were found to be located in the AF3 and DNA binding domain of PR and the RRM (RNA recognizing motif) of PSF. Co-transfection of PSF into myometrial cells resulted in decreased transcriptional activity of PRB, but not of ERα or ERβ. Over-expression of PSF in 293T cells reduced PR protein levels, an effect that could be rescued by the proteosomal inhibitor, MG132. Of significance, we demonstrated a very low level of expression of PSF in the rat myometrium during pregnancy but a dramatic increase near term with maximal levels at the onset of labour. Thus, we have defined novel functions of PSF beyond its actions as a pre-mRNA splicing factor. PSF's interaction with the DNA binding domain of PR blocks PR-mediated transcriptional activity. PSF targets PR for degradation through the 26S proteosome pathway, possibly by interacting with ubiquitin ligases. Together with the increased myometrial expression of PSF at term, these data suggest that PSF may act to induce of a functional withdrawal of progesterone and initiate labour.

### EXAMPLE 2

Summary

Applicant has identified novel progesterone receptor (PR)-interacting proteins that block the progesterone receptor signaling pathway at term in human pregnancy. One of the PR-interacting proteins was identified as polypyridimine tract-binding protein-associated splicing factor (“PSF”), a RNA splicing factor. The interaction between PR and PSF was confirmed by in vivo and in vitro protein interaction assays. PSF was found to interact with both the PR-A and PR-B isoforms. The interaction domains were found to be located in the AF3 and DNA binding domain of PR and the RRM (RNA recognizing motif) of PSF. Co-transfection of PSF into myometrial cells resulted in decreased transcriptional activity of PRB, but not of ERα or ERβ. Over-expression of PSF in 293T cells reduced PR protein levels, an effect that could be rescued by the proteosomal inhibitor, MG 132. PSF expression increased dramatically in the rat myometrium at term pregnancy in association with reduced levels of myometrial PRA very low level of expression of PSF was found in the rat myometrium during pregnancy but a dramatic increase was found near term with maximal levels at the onset of labour. PSF's interaction with the DNA binding domain of PR blocks PR-mediated transcriptional activity. PSF targets PR for degradation through the 26S proteosome pathway. Together with the increased myometrial expression of PSF at term, these data indicate that PSF acts to induce a functional withdrawal of progesterone and initiate labour.

Experimental Procedures

In an effort to identify cofactors within the myometrium that interact with PR and modulate PR function, glutathion-S-transferase (GST)-PR fusion proteins were used to pulldown protein extracts from myometrium smooth muscle cell lysate. One of the associated proteins identified (PSF) was shown to inhibit the transcriptional activity of PR by mechanisms that involved interference of PR binding to PRE, and, the degradation of the PR protein through the proteasome pathway. Furthermore, the finding that PSF expression increased dramatically in the rat myometrium at term pregnancy in association with reduced levels of myometrial PR, has led to the conclusion that this novel PR co-repressor contributes to the functional withdrawal of progesterone and the initiation of labor.

Materials: DNA restriction and modification enzymes were obtained from Fementas (Burlington, ON), Promega (Nepean, ON), Boehringer Mannheim, (Laval, QC). PCR reagents were obtained from Invitrogen (Burlington, ON). Progesterone, 17-β estradiol and the proteasome inhibitor MG132 were from Sigma (Oakville (ON). PR (C-20 and AB52) primary antibody, anti-His tag antibody (H-15), and anti-Gal4 DBD antibody (sc-4050) were purchased from Santa Cruz, Calif., and the anti-PSF antibody (B92) was obtained from Sigma. Protease inhibitor cocktail was purchased from Boehringer Mannheim. Glutathione-Sepharose 4B affinity matrix was from Pharmacia (Oakville, Canada).

Plasmid construction: The PR expression vector pSG5 PRA and PRB were provided by Dr. P Chambon (Strasbourg, France). Using pSG5 PRB as template, a series of deletion mutations (amino acids 1-164, 164-456, 456-556, 456-650,556-650,556-933 and 650-933 of PRB) were generated by polymerase chain reaction (PCR) with 5′ primer containing ERI site and ATG start codon, and 3′ primer containing TGA stop codon and Sallsite, using the Platinum Taq DNA Polymerase High Fidelity (Invitrogen). PCR fragments were then inserted into expression vectors (pM, VP16- Clontech and pGEX 5X-2—Amersham Phamacia). PR 556-650 was also inserted downstream of T7 promoter of pcDNA3 at the site of ERI and XhoI. Flag-PRB was constructed using pFlag-CMV2 (Sigma) as backbone. Full length PR cDNA was amplified by PCR and cloned into CMV-flag at EcoRI and BamHI in frame. Human PSF complementary DNA was from Dr J G Patton (Nashville, Tenn.). Full length and deletion mutants of PSF (amino acid 1-707, 1-662, 1-150, 1-290, 150-290, 290-370, 370-450, 290-707, 370-707, 450-707, 662-707) were also generated by PCR with 5′ primer containing ERI site and ATG start codon, and 3′ primer containing TGA stop codon and SalI site. PCR fragments were then inserted into vectors of pM, pGEX 5X-2 respectively at the site of ERI and SalI, and pcDNA3.1 6×His at the site of ERI and XhoI. Veracity of all PCR generated fragments was confirmed by DNA sequencing. Expressed proteins were also detected by Western Blotting using specific antibodies. ERα and ERβ expression vectors were obtained from Dr. Paul Walfish (Toronto, Canada). The construction of mouse mammary tumor virus—Luc (MMTV-Luc), and 3×ERE-Luc reporter vectors was described previously (20)
Identification of interacting proteins with mass spectrometry: GST fusion proteins were prepared as previous described (20). Briefly, GST fusion proteins produced in E. coli strain BL21(DE3)pLysS cells by incubating with isopropyl-β-D-thiogalactoside to a final concentration of 0.2 mM. Bacteria were pelleted and resuspended in NETN buffer (0.5% NP-40, 1 mM EDTA, 100 mM NaCl, 20 mM Tris pH 8.0) plus protease inhibitor cocktails (Boehringer Mannheim) and lysed by mild sonication. Centrifugation cleared lysate were incubated with 200111 of a 50% slurry glutathione-Sepharose 4B affinity matrix.

Cytoplasm and nuclear fractions of Syrian hamster myocyte (SHM) cells were prepared by NE-PER Nuclear and Cytoplasmic Extraction Kit (Pierce, Rockford, Ill.). Protein extracts (about 1.5 mg) were first pre-cleared by passing through GST bound glutathione-Sepharose 4B matrix and then incubated with GST-fusion proteins bound to sepharose beads for 2 hour at 4° C. The beads were then washed three times with NETN buffer and once with NETN buffer containing 100, 150 or 200 mM NaCl. The associated proteins were eluted by adding 20 mM glutathion and separated on 10% SDS-PAGE. Gels were stained with Coomassie Blue.

Bands were excised, reduced, allcylated and digested in-gel with trypsin as described (21). Tryptic peptides were extracted from the gel, desalted using ZipTip desalting columns (Millipore, Bedford, Mass.), equilibrated in 5% formic acid, washed with equilibration buffer and eluted in a solution of 5% formic acid and 60% methanol. Tandem mass spectrometry analysis was performed by using a nano-electrospray ionization (ESI) source (Protana A/S) coupled to a high-performance hybrid quadrapole time-of-flight API QSTARTM Pulsar mass spectrometer (MDS-Sciex, Concord, ON, Canada). After tryptic ion candidates were identified, product ion spectra were generated by collision-induced dissociation. For product ion scans, the collision energy was determined experimentally. Sequence and mass information of the peptides was used to screen the NRDB and dbEST databases. Databases were searched using the Mascot MS/MS search engine (Matrix Science, London, UK).
Immunoprecipitation and Western Blot: Coimmunoprecipitation of transfected PR and His-PSF was performed in 293T cells. Cells were plated at 150 mm diameter dish and grown to 60% confluent before transfection. Total of 15 μg plasmid was transfected by Exgen 500 (Fermantas). Eighteen hours after transfection, cells were washed two times ice-cold phosphate-buffered saline (PBS) then lysed in NETN buffer plus protease inhibitor cocktail. Protein concentration of the whole cell lysate (WCL) was determined by the Bradford assay and the WCL was diluted to 1 mg/ml in NETN. A 900 μl aliquot of WCL was incubated for overnight at 4° C. in suspension with either anti-His tag or anti-PR antibody, followed by the addition of 30 μl of protein A/G PLUS-agarose beads (Santa Cruz) for another two hours at 4° C. Resins were washed with NETN and eluted with 1×Laemmli buffer, boiled, and centrifuged. The supernatant was separated by SDS- 8% PAGE, electrophoresed to PVDF membrane, and visualized by ECL.

For immunoprecipitation of the endogenous PR and PSF, T47D cells cultured in 150 mm dishes were lysed in 800 μl NETN buffer containing 150 mM NaCl plus protease inhibitor cocktail. Cell lysate were then incubated with 5 μg of PR or PSF, or control mouse IgG at 4° C. overnight, followed by the addition of 30 μl of protein A/G PLUS-agarose beads (Santa Cruz) for another two hours at 4° C. Resins were washed with NETN containing 250 mM NaCl and eluted with 1×Laemmli buffer. The eluted proteins along with the whole cell extract were western blotted by PR or PSF antibody.
GST pulldown assay: GST pulldown assay was performed as previously described (20). GST and its fusion proteins were first immobilized to glutathione-Sepharose 4B affinity matrix. The matrix was then incubated at 4° C. overnight with rabbit reticulocyte lysate (Promega) containing PR or His-PSF transcribed and translated in presence of ³⁵S methionine. The matrix was washed three times with cold NETN buffer before adding 1 ×Laemmli buffer to elute associated proteins. The eluted proteins were separated on 10% SDS-PAGE gel. Gels were treated with Enhancer (NEN, Boston, US) dried and analyzed by autoradiography.
Cell culture and transient transfection: SHM cells and 293T cells were maintained in DMEM plus 5% FCS (Sigma) as described before (22). For experiments involving steroid exposure, the medium was substituted with phenol red free DMEM containing 5% charcoal-treated fetal bovine serum (Hyclone, Utah, USA). Transfection was performed according to manufacturers protocol (Fementas). Cells were seeded at a density to achieve 60-80% confluency the following day. The DNA and transfection reagent were mixed and added to the medium. Cell lysates were collected at least 30 hours after transfection. For luciferase assay, cells were collected in 200 μl of lysis buffer (Promega) of which 10μl was used for the luciferase and β-Gal activity assays respectively. Luciferase activity was determined by using the luciferin reagent (Promega) according to manufacture's protocol. Transfection efficiency was normalized to β-galactosidase activity. For western blots, cell lysates were collected in NETN buffer plus protease inhibitor cocktail. About 30 μg of protein extract was separated on SDS gel electrophoresis followed by western blotting with antibodies of interest.
Electrophoretic Mobility Shift Assay: PR_{DBD} was synthesized in rabbit reticulocyte by using the TNT coupled in vitro transcription-translation system (Promega) with the vector pcDNA3 PR_{DBD}. Full-length PR protein extract was obtained by transiently transfecting 293T cells with Flag-PRB vector. Cells were treated with 10⁻⁸ M progesterone and nuclear fraction was extracted by the NE-PER Nuclear and Cytoplasmic Extraction Reagents Kit (Pierce). The nuclear fraction containing PRs was confirmed by western blot with the PR antibody AB-52 and used in gel shift assay. Double strand synthetic oligonucleotide probe containing a 27 bp perfect palindromic consensus PRE was labeled with [³²P] dATP, and purified by passing through Quick Spin Oligo Columns from Roche (Indianapolis, USA). Binding reactions were performed in a total volume of 20 μl in 1×reaction buffer (5% glycerol, 5 mM dithiothreitol, 5 mM EDTA, 250 mM KCl, 100 mM HEPES (pH 7.5), 1 μg of poly(dI-dC), 25 mM MgCl₂, 1 mg of bovine serum albumin per ml, 1 μg of salmon sperm DNA, 0.05% Triton X-100), 0.5 ng of labeled probe, and in vitro translated receptor protein. In some cases, bacteria expressed GST or GST PSF was added as indicated. The binding reaction was allowed to proceed for 20 minutes at room temperature. The supershift was performed by adding 0.5 μg PR antibody for an additional 45 mins. The reaction mixtures were loaded onto a 5% (60:1) nondenaturing polyacrylamide gel. After 2 h of electrophoresis in 0.5×Tris-borate-EDTA (TBE) buffer at 4° C., the gels were dried and autoradiographed.
   Tissue collection and Northern Blot: Wistar rats (Charles River Co., St. Constance, Canada) were housed individually under standard environmental conditions (12 hr light, 12 hr dark cycle) and fed Purina Rat Chow (Ralston Purina, St. Louis, Mo.) and water ad libitum. Female virgin rats were mated with male Wistar rats. Day one of gestation was designated as the day a vaginal plug was observed. The average time of delivery under these conditions was during the morning of day 23. The criteria for labour were based on delivery of at least one pup.

Rats were killed by carbon dioxide inhalation and myometrial samples were collected on gestational days of 6, 12, 15, 17, 19, 21, 22, 23, or 1 and 4 days postpartum (1 PP and 4 PP). Tissue was collected at 10 a.m. on all days with the following exceptions: the labour (d23L) sample was collected once the animals had delivered at least one pup (n=5). Rat myometrial tissues were placed into ice-cold phosphate buffered saline (PBS), bisected longitudinally and dissected away from both pups and placentas. The endometrium was carefully removed from the myometrial tissue by mechanical scraping on ice. This was previously shown that this removes the entire luminal epithelium and the majority of the uterine stroma (23). The myometrial tissue was flash-frozen in liquid nitrogen. All other tissues from female and male animals (ovary, placenta, heart, liver, lung, small intestine, brain, kidney, skeletal muscles and testicles) were collected at the same time and flash-frozen in liquid nitrogen. All tissues were stored at −70° C.

Total RNA was extracted from the tissues using TRIZOL (Gibco BRL, Burlington, ON). Northern blot and hybridization were carried out as described (24). The probe used to detect PSF mRNA was a 770 fragment generated by PCR encompassing sequence from 1436 to 2209 (Genbank No. X70944). The 18S probe provided by Dr. David T. Denhardt (Rutgers University NJ) was used as a control probe. A total of five sets of gestational profiles were subjected to a one-way ANOVA followed by pairwise multiple comparison procedures (Student-Newman-Keuls method) to determine differences between groups, with the level of significance for comparison set at P<0.05.

The expression of PR protein was determined by western blotting. Frozen tissue was crushed under liquid nitrogen using a mortar and pestle. Crushed tissue was homogenized for 1 minute in RIPA lysis buffer [50 mM Tris-HCl pH 7.5, 150 mM NaCl, 1% (vol/vol) Triton X-100, 1% (vol/vol) sodium deoxycholate, 0.1% (wt/vol) SDS, supplemented with 100 μM sodium orthovanadate and protease inhibitor cocktail tablets (Complete™ Mini; Roche, Quebec, Canada)]. Samples were spun at 12000 g for 15 min at 4° C. and the supernatant was transferred to a fresh tube to obtain a crude protein lysate. Protein concentrations were determined using the BioRad protein assay buffer (BioRad, Hercules, Calif.). Protein samples (40-50 μg) were resolved by electrophoresis on an 8% SDS-polyacrylamide gel. Proteins were transferred onto polyvinylidene difluoride (PVDF) membrane (Millipore, Bedford, Mass.) in 25 mM Tris-HCl, 250 mM glycine, 0.1% (wt/vol) SDS, pH 8.3 for 18 hr at 30 mV at 4 C, blotted with anti-PR antibody C-20, exposed to x-ray film (Kodak XAR, Eastman Kodak, Rochester, N.Y.) and analyzed by densitometry. The membrane was then stripped and blotted with anti-calponin antibody as a loading control. Four complete sets of gestational profiles were analyzed by western blotting and the data were subjected to a one-way ANOVA followed by pairwise multiple comparison procedures (Student-Newman-Keuls method) to determine differences between groups, with the level of significance for comparison set at P<0.05.

Results

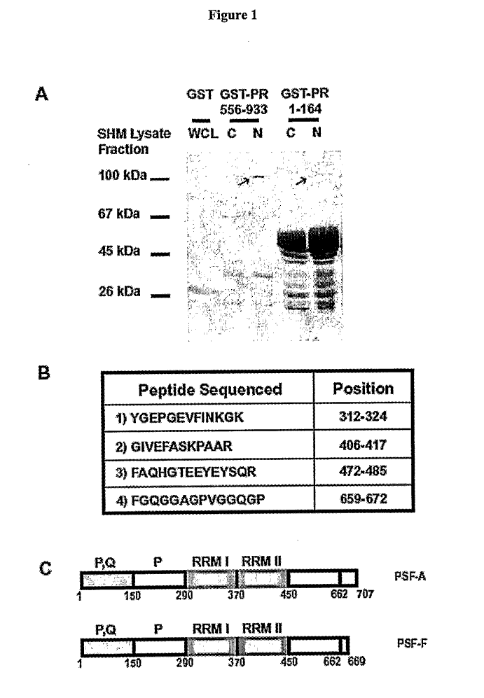

Identification of PSF as a PR interacting protein. In order to identify PR interacting proteins, GST-PR fusion proteins were bound to glutathione-Sepharose 4B matrix and incubated with either cytosol or nuclear extracts from SHM cells pre-cleared by passing through GST bound glutathione-Sepharose 4B matrix. Associated proteins were resolved on an SDS gel, and visualized with Coomassie blue stain. Two protein bands, present only in nuclear fractions of SHM cells, were identified that migrated at the same molecular weight of 100 kDa and bound to PR₁₋₁₆₄ and PR₅₅₆₋₉₃₃ respectively (FIG. 1A). GST-PR₅₅₆₋₉₃₃ was found to bind p100 at a wide range (100 mM, 150 mM and 200 mM) of NaCl concentrations, whereas, GST-PR₁₋₁₆₄ could only bind p100 at a concentration of 150 mM NaCl. This suggests that p100 may form a more stable complex with GST-PR₅₅₆₋₉₃₃

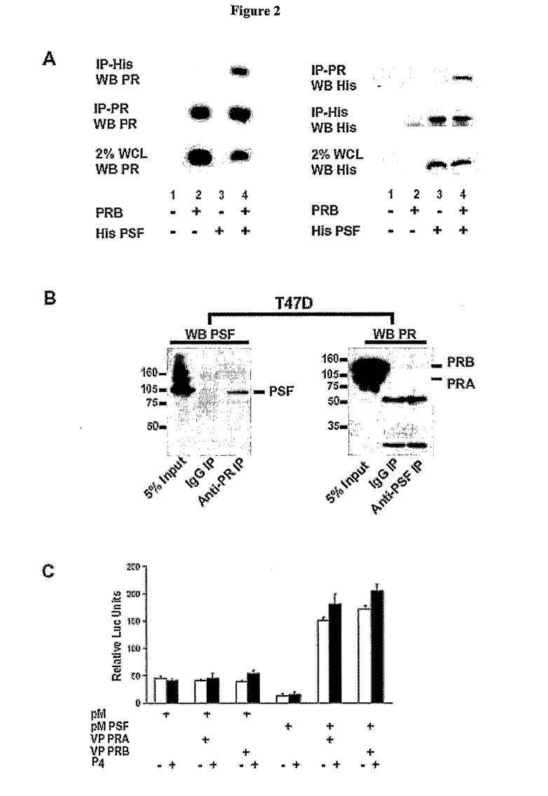

These two p100 bands were excised and processed for MALDI mass spectrometry. Four peptide sequences within the proteins were identified; these data indicated that these two p100 proteins are identical (FIG. 1B). The sequences matched perfectly within the BLAST database to a known protein termed PSF (polypyrimidine tract binding protein-associated splicing factor), previously identified as a RNA splicing factor. Two PSF protein isoforms have been reported, designated as PSF-A and PSF-F, respectively (FIG. 1C). These two isoforms are identical through amino acids 1-662 but thereafter diverge, with PSF-F containing 669 amino acids and PSF-A containing 707 amino acids (25). PSF contains two RNA recognition motifs (RRM I and II, within amino acids 290 to 450) and an unusual N-terminal region rich in proline and glutamine residues and appears to migrate anomalously as a 100-kDa protein in SDS-gels. The MALDI mass spectrometry analysis did not detect any sequences specific to PSF-F, but the peptide “FGQGGAGPVGGQGP” [SEQ ID NO. 16 ] did match specifically to PSF-A.
Confirmation of PSF interaction with PR in vivo. Immunoprecipitation was used to confirm the interaction between PR and PSF in vivo. His-PSF was constructed by insertion of the PSF open reading frame (from PSF cDNA provided by Dr. J Pattern) into the C-terminus of 6×His tag. 293T cells were transient transfected with expressing plasmid of PR and/or His-PSF as indicated (FIG. 2A).

Whole cell lysates (WCL) were first western blotted with PR and His-PSF antibody to ensure these two proteins were appropriately expressed in the cells (bottom of FIG. 2A). Inmmunoprecipitation with either PR or His-PSF antibody was performed followed by western blotting with the same antibody to ensure that target proteins could be precipitated by the protein A/G PLUS-agarose beads (middle of FIG. 2A). Finally, whole cell extracts expressing PRB and/or His tagged PSF were incubated with PR antibody and then incubated with protein A/G PLUS-agarose beads. The associated proteins were washed and analyzed for the presence of His-PSF. Overexpressed His-PSF was specifically co-immunoprecipitated only in the presence of PR (top of FIG. 2A). Similarly, when His-tag antibody was used to immunoprecipitate whole cell lysate, association of PR could only be detected in the presence of His-PSF. Endogenous PSF was also coimmunoprecipitated with endogenous PR from T47D cell extract (FIG. 2B). No immunoprecipitation of PR or PSF was observed when anti-PSF or anti-PR antibody was replaced by control mouse IgG. These in vivo data confirm the interaction between PR and PSF found with the GST pulldown experiment.

Further evidence to support an in vivo interaction between PSF and PR was gained by using the mammalian two-hybrid system (FIG. 2C). PSF was fused to C-terminus of Gal4-DBD in pM vector, while PRB or PRA was fused downstream of Gal4 activation domain in VP16 vector. When co-transfected with G5-Luc, pM-PSF resulted in a 70% reduction in luciferase activity compared to pM empty vector. However, co-transfection of both activation domain tagged PRs (VP16-PRA and VP16-PRB) with pM-PSF induced a dramatic increase of luciferase activity as a result of the interaction between PSF and PRs. This interaction is ligand-independent, since addition of progesterone did not cause a significant difference in luciferase activity. These data provide further evidence that PR and PSF interact in vivo.

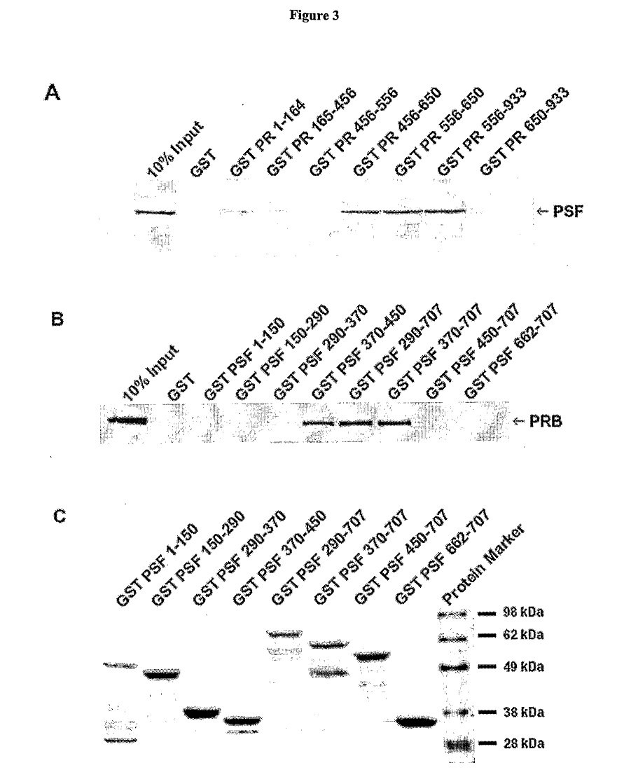

Mapping interacting sites within PR and PSF. To determine whether the interaction between PSF and PR is direct, and if so, to define the physical location of the interaction sites, a GST in vitro pulldown assay was performed. PSF was first ³⁵S-labeled by coupled in vitro transcription/translation, and its binding to a series of GST-PR fusion proteins was assessed (FIG. 3A). PSF bound strongly to GST-PR fusion proteins containing the DBD domain and to a lesser extent to the AF3 domain of PRB, but did not bind other segments of PR. Further experiments were also carried out to assess direct binding of full length PR to GST-PSF fusion proteins containing proline/glutamine rich domain, RRM I, RRM II and the C-terminus respectively. Specific binding of ³⁵S-labeled PRB was only detected with GST-PSF fusion proteins containing the RRM II domain (FIG. 3B). These differences in PRB binding to GST-PSF fusion proteins were not due to different inputs of GST fusion proteins since electrophoresis of the same mass of GST-PSF fusion proteins produced similar densities of protein bands (FIG. 3C). These data indicate that interaction between PR and PSF is direct and mediated through the AF3 and DNA binding domains of PR and the RRM II domain of PSF.

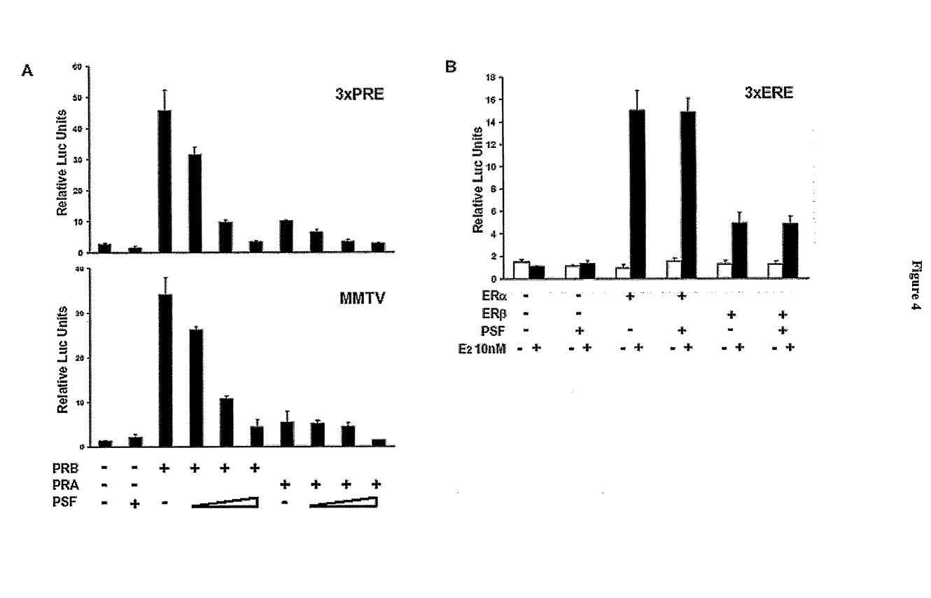

Functional consequence of the interaction between PSF and PR. The impact of the interaction between PSF and PR on the transcriptional transactivation of progesterone responsive promoters was next investigated in SHM cells (FIG. 4A). Two promoter contexts were compared: the MMTV promoter and artificial 3×PRE linked upstream of the luciferase reporter gene. Transient transfection of PSF alone had no effect on either of these promoters. However, PSF potently inhibited both PRA and, even more dramatically PRB, transactivation of both promoters in a dose dependent manner. Similar inhibitory effects were also observed in the experiments performed in 293T cells (data not shown), indicating that this effect is not dependent upon the cell or promoter context. The PSF expression vector was also co-transfected with ERα and ERβ in SHM cells (FIG. 4B) in a 3×ERE promoter context. Inhibitory effects of PSF on either ERα or ERβ were not observed, indicating that PSF selectively represses to PRs rather ERs.

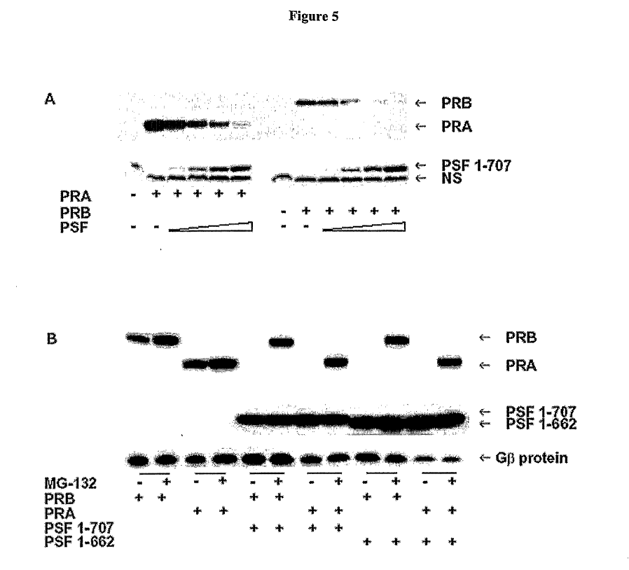

PSF enhances the degradation of PR protein through proteasome pathway. To exclude the possibility that the inhibitory effects of PSF on PR were not due to reduced expression of PR proteins, western blots of whole cell lysates were conducted following co-transfection of PSF and PR expression vectors as described above in FIG. 4. Increasing the dose of PSF expression vector input (in the presence of constant dose of PR expressing vector) resulted in decrease in the level of both PRA and PRB protein (FIG. 5A). This interesting observation led to investigation of the possibility that PSF enhances the degradation of PR through the 26S proteasome pathway. Co-transfection of PSF or PSF-₁₋₆₆₂ (a truncated form of PSF lacking the PSF-F specific C-terminal domain) and PRB/PBA resulted in a total loss of PR proteins after 24 hours (FIG. 5B). However, addition of the proteasomal inhibitor, MG-132 (Sigma), at a final concentration of 60 μM for an additional 6 hours block the loss of PR protein. PSF₁₋₆₆₂ also enhanced degradation of PR proteins implying that this region of PSF is sufficient to degrade PR and that the PSF-F isoform would also have this capability.

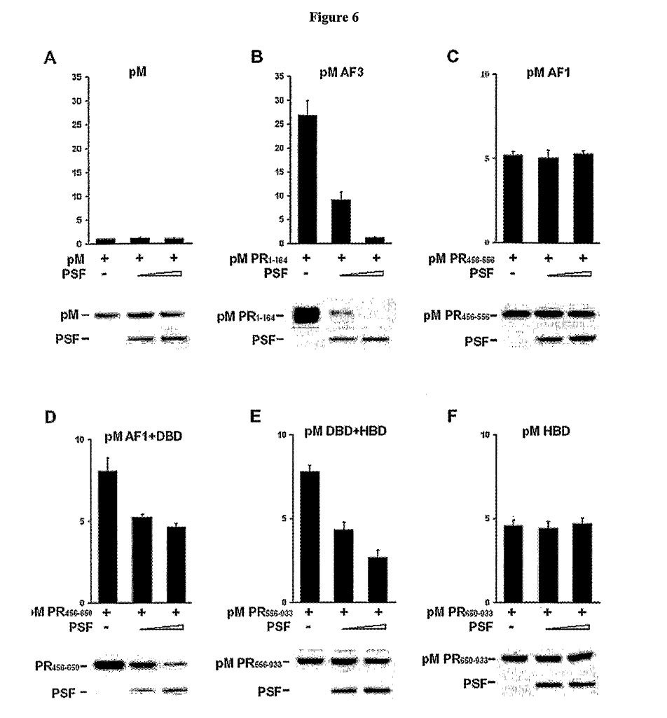

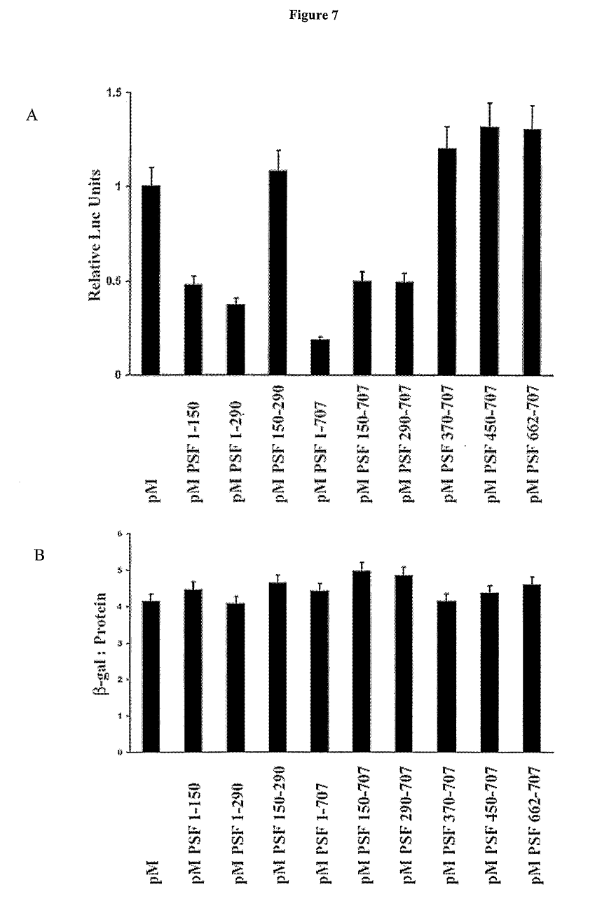

PSF represses PR transactivation domains through two different mechanisms. To investigate the repression of PSF on individual activation domains of PR, a one-hybrid system was used, in which each of the activation domains of PR were fused to the C-terminus of Gal4 DBD in the pM vector and co-transfected with G5-Luc reporter gene. PR₁₋₁₆₄ induced a 25-fold increase in luciferase activity compared to the empty pM vector (FIG. 6B). Co-transfection of increasing amounts of PSF resulted in a significant dose-dependent reduction in luciferase activity coupled with a loss of pM PR₁₋₁₆₄ protein. PSF also inhibited transactivation and enhanced degradation of PR₄₅₆₋₆₅₀ (a region containing both AF1 domain and DBD). However, PSF had no effect on PR₄₅₆₋₅₅₆ (FIGS. 6C and 6D), possibly because PR₄₅₆₋₅₅₆ (AF1 domain of PR) lacks a binding site for PSF, nor on PR₆₅₀₋₉₃₃ (containing only the AF2 domain) (FIG. 6F). Importantly, PSF did inhibit transactivation of PR₅₅₆₋₉₃₃ (containing the DBD and AF2 domain), without any reduction in the protein level of this domain (FIG. 6E). These data suggest that the ability of PSF to inhibit transactivation and induce degradation of the AF3 and AF1 is dependent upon the physical interaction of PSF with AF3 and DBD. On the other hand, although PSF also directly interacts with PR₅₅₆₋₉₃₃, inhibition of the transactivation function of this domain involves a separate mechanism distinct from degradation. Potential mechanisms might include repressor domains within PSF or the recruitment of other transcriptional inhibitory protein complexes. To explore this possibility, a one-hybrid system was set up in which various segments of PSF cDNA were inserted into pM vector in frame and cotransfected with the G5-luciferase and pCH110 reporter genes. Two regions (aa 1-150, 290-370) within PSF that were responsible for the inhibitory effects were identified (FIG. 7A). The β-galactosidase activities normalized by protein concentrations confirmed that the inhibitory effects were not due to generalized effect of PSF overquenching gene expression (FIG. 7B).

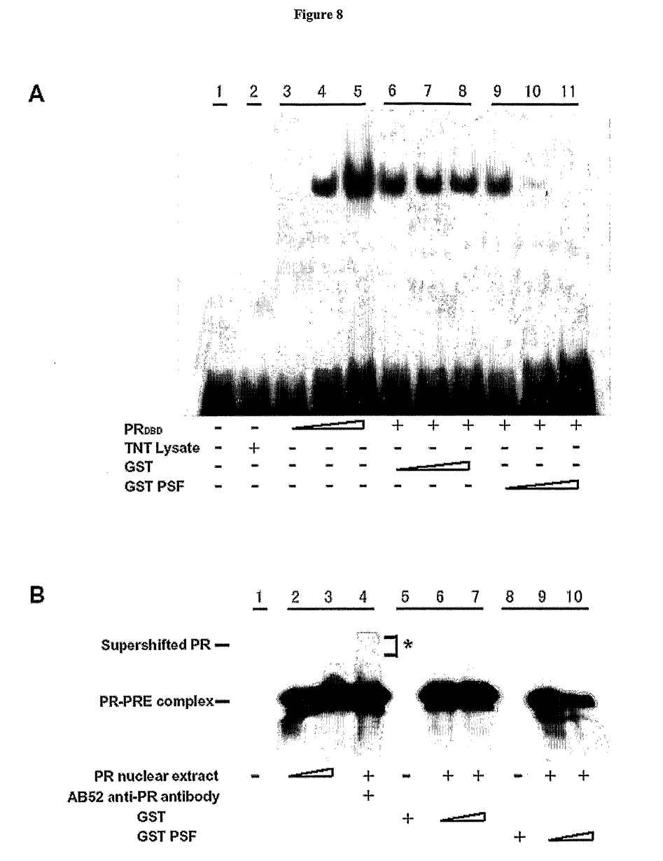

PSF disrupts interaction between PR DBD and PRE. The finding that the inhibition of PR₅₅₆₋₉₃₃ transactivation function by PSF is not due to degradation of this PR domain suggests that multiple mechanisms are involved in the co-repression of PR signaling by PSF. The direct interaction between PSF and PR_{DBD} raises the possibility that PSF could block PR_{DBD} binding to its response element. To address this question, Electrophoretic Mobility Shift Assay was performed to study the interaction between PR_{DBD} and PRE. As shown in FIG. 8A, TNT coupled in vitro transcribed-translated PR_{DBD} formed a complex with ³²P-labeled double strand oligonucleotide of PRE (lane 3 to 5) in a dose dependent manner. This interaction is specific since TNT plain lysate did not form complexes with the PRE oligo (lane 2). Addition of GST-PSF fusion protein into the reaction resulted in decreased association of PR_{DBD} to PRE (Lane 9 to 11), whereas GST protein alone had no effect on this interaction (lane 6 to 8). To further valid the above observation, a gel shift assay was performed with full-length PR obtained from nuclear extraction of 293T cells transient transfected with Flag-PRB (FIG. 8). PR forms complex with PRE oligo in a dose dependent manner (lane 2 and 3). To clarify the molecular composition of the shifted band, anti-PR antiserum was added to the incubation mixture. The band was supershifted by the addition of anti-PR antibody (lane 4). Note that the supershift band is always diffuse and multiple supershifted bands were observed. Adding increasing doses of GST-PSF but not GST resulted in a decrease of association of PR to PRE (lane 6, 7 and 9, 10). In the control experiments (lane 5 and 8), GST or GST-PSF incubated with PRE did not change the migration pattern of the PRE. Thus, blockade of PR_{DBD} binding to PRE represents an additional mechanism by which PSF can repress PR signaling.

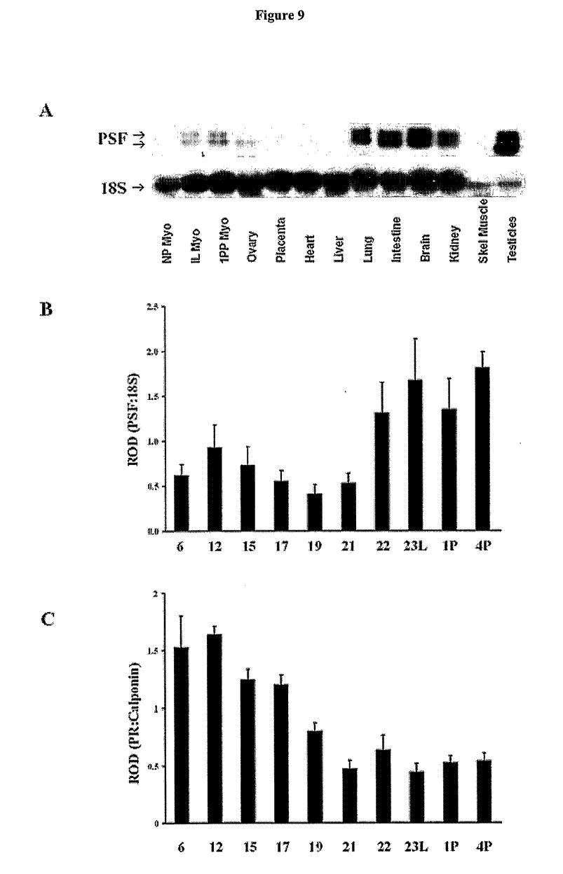

PSF mRNA is widely expressed and upregulated in the myometrium prior to the onset of labor. The tissue distribution of PSF was assessed in rat tissues by Northern Blot analysis. PSF expression was detected in the myometrium and other tissues as two major transcripts of approximately 2.5 and 3.0 kb in length; the relative expression of these two bands was tissue dependent (FIG. 9A). PSF was highly expressed in brain, testis and intestine, at intermediate levels in lung, kidney, ovary and placenta and at low but still detectable levels in liver, skeleton muscle and non-laboring myometrium. Interestingly, expression of PSF was higher in the myometrium from laboring and postpartum samples compared to the myometrial samples from non-pregnant animals.

To more fully define the gestational control of PSF expression, myometrial tissue was collected from pregnant rats at gestational days 6, 12, 15, 17, 19, 21, 22, 23L (labour) and 1 and4 days postpartum (n=5 at each time point) for Northern blot analysis. Densitometric analysis revealed that myometrial PSF mRNA was relatively low throughout early and mid pregnancy, but increased markedly on day 22 (prior to labor) and remained elevated during labor (day 23) and the immediate postpartum period (FIG. 9B). Analysis of variance revealed significant change in PSF expression during pregnancy (p=0.03), with levels after day 22 being significantly higher than early in gestation (day 6) (P<0.05).

Temporal association between increased expression of PSF and reduced PR levels in term myometrium. Based on the gestational expression profile of PSF and its ability to degrade PR in vitro, the increase PSF near term should correlate with reduced myometrial PR levels. To test this myometrial tissues were collected from pregnant rats at gestational days 6, 12, 15, 17, 19, 21, 22, 23L (labour) and 1 and4 day postpartum (n=4 at each time point) for Western blot analysis of PR expression. As predicted densitometric analysis (FIG. 9C) demonstrated an inverse relationship between PSF and PR expression with relatively high levels of PR throughout pregnancy (when PSF is low) with a significant decrease with the approach of term (as PSF expression increases) (P<0.01). These in vivo data are thus consistent with a model in which PSF induces a functional withdrawal of progesterone at term and that at least one mechanisms involves targeting PR for degradation through the proteasomal pathway as well as a possibly action through blockade of PR binding to DNA.

Discussion

The study identified PSF as a novel progesterone-receptor interacting protein that is able to block progesterone signaling. Importantly, the increased myometrial expression of PSF near term coupled with reduced levels of PR suggests that PSF may be a critical contributor to a functional withdrawal of progesterone and initiation of onset labour.

PSF was first cloned and characterized by Dr. James Patton (25) and has been shown to be an essential component of the RNA splicing machinery within the cell (26). PSF forms complexes with high-molecular mass assembly of small nuclear ribonucleoproteins (snRNP) particles and other splicing factors of the SR and hnRNP families (27). Studies have demonstrated that PSF controls the expression of genes involved in cellular differentiation in higher eukaryotes, through regulation of mRNA maturation (28, 29). Evidence was provided that during pregnancy myometrial cells undergo a program of cellular differentiation culminating in a contractile phenotype that transforms (“activates”) the relatively quiescent myometrium to a tissue capable of generating the intense, synchronous contractions of labour (1). This switch in myometrial phenotype is usually triggered by a fall in plasma levels of progesterone, which is able to suppress the expression of genes within the myometrium that are required for labour. While progesterone withdrawal, as a mechanism for labour initiation, has been a widely accepted paradigm for many decades, there are several aspects that suggest that it may not fully account for the dramatic changes seen during labour. Firstly, while progesterone levels do indeed fall in virtually all species at term, the concentration at the initiation of labour remains at a sufficiently high level (e.g. 15-40 nM in rodents) that it would be expected to saturate the myometrial progesterone receptors (Kd=1 nM) (30). Secondly, and more importantly, progesterone levels do not fall prior to labour in the human but are maintained at pregnancy levels. Nevertheless, as in other species, in the human progesterone is required for maintenance of pregnancy and blockade of progesterone signaling leads to the initiation of labour. Thus, administration of the progesterone receptor antagonist, RU486, to pregnant women results (as it does in animals) in the induction of labour (31). These observations have led to the suggestion that a functional withdrawal of progesterone is a pre-requisite for the initiation of human labour. The data presented here indicate that PSF, in addition to regulating gene expression at the level of pre-mRNA splicing, can also function as a co-repressor of the progesterone receptor, thereby removing the suppressive action of this nuclear transcription factor on the expression of myometrial genes required for labour.

Accumulating evidence has suggested links between pre-mRNA splicing and gene transcription. Thus, p54(nrb) had been found to interact and co-activate the androgen receptor (AR) AF-1 within a complex including PSF, paraspeckle protein 1 (PSP 1), and PSP2, which modulate pre-mRNA processing (32). In addition, PGC- 1, which was originally identified as a transcriptional co-activator of the nuclear receptor PPARγ and of several nuclear receptors (33 and 34), has been shown to interact with components of the splicing machinery, therefore allowing coordinated regulation of both transcription and splicing (35). Also, ANT-1 (containing elements of sequence identity to a protein that binds to the U5 small nuclear ribonucleoprotein particle involved in the spliceosome) enhances the ligand-independent autonomous AF-1 transactivation function of AR or glucocorticoid receptor (GR) but does not enhance that of estrogen receptor alpha (36). Other RNA binding proteins (such as RTA, SHARP, p72/p68, TLS and GRIP120) have been shown to either co-activate or co-repress nuclear receptors (37, 38, 39, 40 and 41). Although the detailed molecular mechanisms remain under investigation, evidence indicates an involvement in the recruitment of histone acetylase or histone deacetylase, which in turn regulates chromatin condensation. Samuels and colleagues have reported that PSF can interact with Sin3A and mediates transcriptional silencing through the recruitment of histone deacetylase to the thyroid receptor DBD (42). The data herein indicate additional mechanisms by which PSF might co-repress PR transactivation, namely enhancement of degradation of PR protein through the proteasome pathway as well as the interference of PR binding to PRE.

Cotransfection of PSF and PR resulted in decreased PR protein and this effect could be reversed by the adding proteasome inhibitor MG132, suggesting that PSF can mediate PR protein degradation through proteasome pathway. PSF itself may possess E3 ubiquitin ligase activity or can bridge PR with protein complexes containing this similar function. The observation that the truncated form of PSF (PSFI-662) can degrade PR indicates that both PSF-A and PSF-F possess PR degradation capabilities. The different localization of PSF-F and PSF-A to cytoplasmic and nuclear compartments respectively indicates that PSF-F could target PR for degradation as early as protein synthesis in Golgi Apparatus and endoplasmic reticulum, while PSF-A would target the nuclear receptor. This in vitro function of PSF in targeting PR for degradation is given increased functional significance by the in vivo data showing a correlation between increased expression of PSF in the rat myometrium at term and reduced level of PR protein.

A second mechanism by which PSF may block PR signaling is through interference of PR DBD binding to PRE as evidenced by the EMSA assay. The data indicate that at least for the AF2 domain of PR, this interference with DNA binding is independent of an effect of PR degradation.

The data also provides evidence that two regions within the N-terminus of PSF possess transcriptional inhibitory effects. This result is consistent with recent published data that the C-terminal truncated form of PSF (not containing RNA recognition motifs) inhibits gene transcription of the P450 cholesterol side chain cleavage enzyme by binding to a DNA sequence ‘CTGAGTC’ [SEQ ID NO.17] within the its promoter (43). Although evidence has been provided that PSF can recruit other transcriptional inhibitory protein complexes through Sin3A (42), the possibility that the N-terminus of PSF possesses intrinsic inhibitory functions cannot be excluded.

The observation that PSF co-represses the transactive function of PR [as well as glucocorticoid receptor (GR), androgen receptor (AR) but not ER (ERα or ERβ)] suggests that this action is relatively selective. Given that the DBD of nuclear receptors has a high degree of similarity (as opposed to that of the N-terminus) it may be that PSF binding to the DBD of PR represents a primary site of interaction. This is supported by the observation that while PSF can interact with PR at both the AF3 domain and the DBD, the affinity of these interactions is rather different with a much stronger binding occurring between PSF and the DBD of PR. This difference may be due to the AF3 domain adopting a more versatile and flexible conformation. Nevertheless, given that PRB (in contrast to PRA) contains an AF3 domain it is possible that PSF may form a relatively stronger interaction with PRB and thus more efficiently co-repress transactivation by PRB compared to PRA.

Thus the data indicate three potential mechanisms by which PSF may co-repress steroid receptors, namely induction of receptor degradation, interference with receptor:DNA binding and recruitment of histone deacetylase (HDAC) protein complexes. The data in the rat suggest an in vivo link between increased PSF expression and PR degradation.

Reports have suggested other potential mechanisms by which a functional progesterone withdrawal might occur in human pregnancy. Several groups have proposed that PR-A can act as a negative repressor of PR-B within the myometrium and that an increase in the relative expression of PR-A versus PR-B might induce a functional withdrawal of progesterone (44 and 3). Conlon et al have reported decreased expression of steroid receptor co-activators in human fundal myometrium at term that might impair PR function (4). Given the critical need for progesterone withdrawal at term, multiple mechanisms may exist to ensure a functional progesterone withdrawal and the initiation of labor.

In summary, a novel function for PSF as a PR co-repressor has been identified. PSF appears to act by blocking the ability of PR to bind to its DNA response element and to target the PR for degradation through the proteasomal pathway. There is a decrease in the binding affinity of PR from term myometrial protein extract to PRE, suggesting that factor(s) present in term myometrium contribute to an inhibition of the interaction between PR to PRE (45). The results indicate that PSF contributes to this blockade. Thus, PSF is a critical component of the mechanism(s) by which a functional withdrawal of progesterone occurs in human pregnancy and it represents a target for therapeutics aimed at controlling the process of labour both at term and preterm.

### EXAMPLE 3

Modulating Androgen Receptor (AR) and Glucocorticoid Receptor

Effect of PSF-A on DHT-induced AR Transactivation in PC-3(AR)₂ Cells

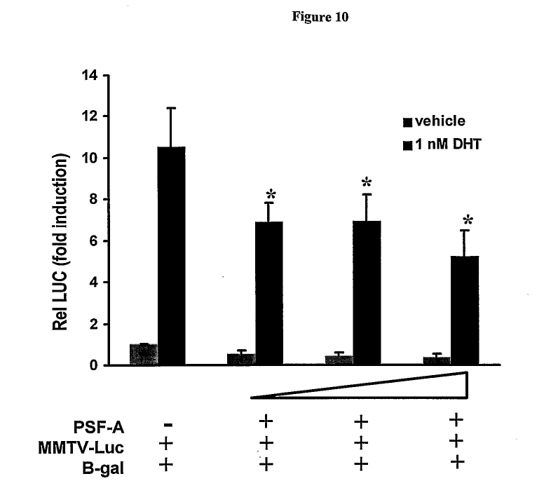

Prostate cancer cells (stable transfected with androgen receptor) were transiently transfected with PSF-A expression vector together with a MMTV-Luc reporter vector. Cells were treated with either vehicle or 1 nM DHT for at least 24 hours. Luciferase activities were measured and normalized by β-galactosidase acitvity. Data shown in FIG. 10 are the mean of three separate experiments performed in triplicate ±S.E. Note: the empty vector pcDNA3 was added to the DNA mixture to ensure that the amounts of CMV promoter in all the transfection assays are equal.

Effect of PSF-F on DHT-induced AR Transactivation in PC-3(AR)₂ Cells.

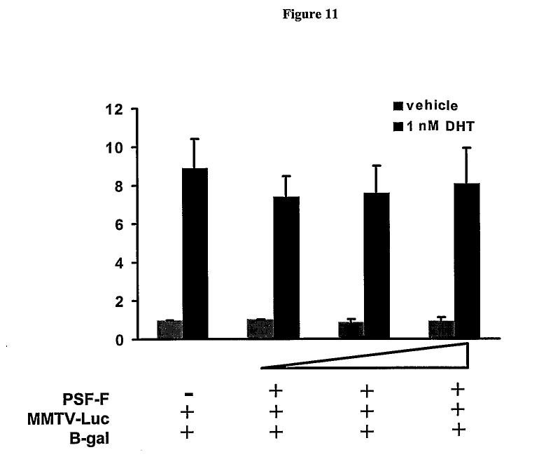

Prostate cancer cells (stably transfected with androgen receptor) were transiently transfected with PSF-F expression vector together with a MMTV-Luc reporter vector. Cells were treated with either vehicle or 1 nM DHT for at least 24 hours. Luciferase activities were measured and normalized by β-galactosidase acitvity. Data shown in FIG. 11 are the mean of three separate experiments performed in triplicate ±S.E. Note: the empty vector pcDNA3 was added to the DNA mixture to ensure that the amounts of CMV promoter in all the transfection assays are equal.

PSF-A & AR Stability in PC-3(AR)₂ Cells

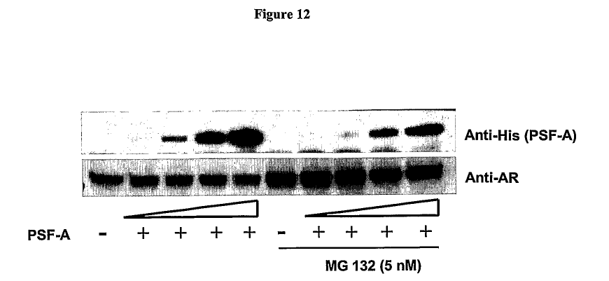

PC-3(AR)₂ cells are PC-3 cells rendered androgen sensitive by stable transfection of a human androgen receptor cDNA. PC-3(AR)₂ cells were transiently transfected with a constant AR expression vector together without or with increasing doses of PSF-A vectors. Cells treated with the proteosomal inhibitor MG132 are shown in FIG. 12. Whole cell lysates were collected and Western Blot was performed.

PSF Inhibits Glucocorticoid Receptor Ttransactivation in SHM Cells

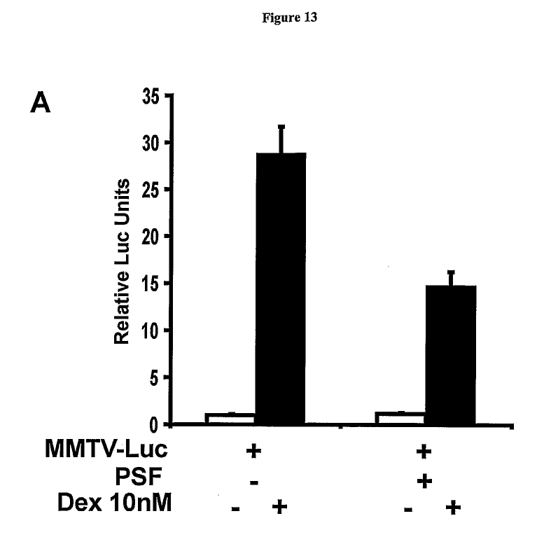

Hamster smooth muscle cells (SHM) were transiently transfected with PSF-A expression vector together with a MMTV-Luc reporter vector. Cells were treated with either vehicle or 1 nM Dex for at least 24 hours. Luciferase activities were measured and normalized by β-galactosidase acitvity. Data shown in FIG. 13 are the mean of three separate experiments performed in triplicate ±S.E. Note: the empty vector pcDNA3 was added to the DNA mixture to ensure that the amounts of CMV promoter in all the transfection assays are equal.

The results showed that PSF interacts with the androgen receptor and blocks both its activation of the MMTV promoter and production/secretion of PSA. The results also showed that PSF interacts with the glucocorticoid receptor and blocks its activation of the MMTV promoter.

The results indicated that PSF functions as a modulator of androgen function acting to decrease receptor transcriptional activity. PSF may also increase androgen receptor degradation. Decreased expression of PSF in prostate represents a mechanism by which prostate cancer cells may acquire enhanced sensitivity to residual androgens or growth factor/cytokine activation of the androgen receptor in the presence of androgen-ablation therapy. Thus, PSF can be used to target the androgen receptor in prostate cancer to decrease or block transactivational activity of the receptor.

The results also indicated that PSF functions as a modulator of glucocorticoid receptor decreasing receptor transcriptional activity.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a polypeptide” includes a plurality of such polypeptides, reference to the “antibody” is a reference to one or more antibodies and equivalents thereof known to those skilled in the art, and so forth.

### FULL CITATION OF REFERENCES

1. Challis J. R. G, Matthews S G, Gibb W, Lye S J. (2000) Endocr Rev. (5): 514-50.
2. Challis J. R. G, Lye S. J (2004) In: Maternal-Fetal Medicine Principles and Practice fifth edition, Eds Creasy R K and Resnik R. pp 79-87 Saunders, Philadelphia 2004
3. Mesiano S, Chan E C, Fifter J T, Kwek K, Yeo G, Smith R (2002) J Clin Endocrinol Metab. 6:2924-30.
4. Condon J C, Jeyasuria P, Faust J M, Wilson J W, Mendelson C R (2003) Proc Natl Acad Sci U S A. 100:9518-23.
5. Kastner P, Krust A, Turcofte B, Stropp U, Tora L, Gronemeyer H, Chambon P. (1990) EMBO J. (5): 1603-14.
6. Tora L, Gronemeyer H, Turcotte B, Gaub M P, Chambon P. (1988) Nature. 333(6169): 185-8.
7. Meyer M E, Quirin-Stricker C, Lerouge T, Bocquel M T, Gronemeyer H. (1992) J Biol Chem. 267(15): 10882-7.
8. Giangrande P H, McDonnell D P. (1999) Recent Prog Horm Res. 54:291-313; discussion 313-4.
9. Vegeto E, Shahbaz M M, Wen D X, Goldman M E, O'Malley B W, McDonnell D P. (1993) Mol Endocrinol. (10): 1244-55.
10. Iraus W L, Weis K E, Katzenellenbogen B S. (1995) Mol Cell Biol. (4): 1847-57.
11. Ing N H, Beekman J M, Tsai S Y, Tsai M J, O'Malley B W. (1992) J Biol Chem. 267(25): 17617-23.
12. Onate S A, Tsai S Y, Tsai M J, O'Malley B W. (1995) Science. 270(5240): 1354-7.
13. Heinzel T, Lavinsky R M, Mullen T M, Soderstrom M, Laherty C D, Torchia J, Yang W M, Brard G, Ngo S D, Davie J R, Seto E, Eisenman R N, Rose D W, Glass C K, Rosenfeld M G. (1997) Nature. 387(6628):43-8.
14. Tsai M J, O'Malley B W (1994) Annu Rev Biochem. 63:451-86. Review.
15. Sartorius C A, Melville M Y, Hovland A R, Tung L, Takimoto G S, Horwitz K B (1994) Mol Endocrinol. 8(10): 1347-60.
16. Tetel M J, Giangrande P H, Leonhardt S A, McDonnell D P, Edwards D P. (1999) Mol Endocrinol. (6): 910-24
17. Glass C K, Rosenfeld M G. (2000) Genes Dev. 14(2): 121-41. Review
18. Nawaz Z, Lonard D M, Smith C L, Lev-Lehman E, Tsai S Y, Tsai M J, O'Malley B W. (1999) Mol Cell Biol. (2): 1182-9.
19. McKenna N J, Xu J, Nawaz Z, Tsai S Y, Tsai M J, O'Malley B W. (1999) Steroid Biochem Mol Miol. 69(1-6): 3-12. Review
20. Dong X, Challis J R G and Lye S J (2004) J Mol Endocrinol. 32(3):843-57.
21. Plant P J, Fawcett J P, Lin D C, Holdorf A D, Binns K, Kulkarni S, Pawson T. (2003) Nat Cell Biol. (4): 301-8.
22. Chen Z Q, Lefebvre D, Bai X H, Reaume A, Rossant J, Lye S J. (1995) J Biol Chem. 270(8): 3863-8.
23. Piersanti M, Lye S J. (1995) Endocrinology. 136(8):3571-8.
24. Shynlova O, Mitchell J A, Tsampalieros A, Langille B L, Lye S J. (2004) Biol Reprod. 70(4): 986-92.
25. Patton J G, Porro E B, Galceran J, Tempst P, Nadal-Ginard B (1993) Genes Dev. 7(3): 393-406.
26. Iramer A (1996) Annu. Rev. Biochem. (65); 331-409
27. Will C L, Luhrmann R. (1997) Curr. Opin Cell Biol (9); 320-328
28. Dreyfuss G, Matunis M J, Pinol-Roma S, Burd C G. (1993) Annu Rev Biochem. 62:289-321. Review.
29. Manley J L, Tacke R. (1996) Genes Dev. 10(13): 1569-79. Review.
30. Pepe G. J., Rothchild I. (1974) Endocrinology 95: 275-279
31. Neilson J P. (2000) Cochrane Database Syst Rev. 4:CD002865
32. Ishitani K, Yoshida T, Kitagawa H, Ohta H, Nozawa S, Kato S. (2003) Biochem Biophys Res Commun. 306(3): 660-5.
33. Puigserver P, Adelmant G, Wu Z, Fan M, Xu J, O'Malley B, Spiegelman B M (1999) Science. 286(5443): 1368-71.
34. Tcherepanova I, Puigserver P, Norris J D, Spiegelman B M, McDonnell D P (2000) J Biol Chem. 275(21): 16302-8.
35. Knutti D, Kralli A (2001) Trends Endocrinol Metab. 12(8): 360-5. Review.
36. Zhao Y, Goto K, Saitoh M, Yanase T, Nomura M, Okabe T, Takayanagi R, Nawata H (2002) J Biol Chem. 277(33): 30031-9.
37. Norris J D, Fan D, Sherk A, McDonnell D P (2002) Mol Endocrinol. 16(3):459-68.
38. Shi Y, Downes M, Xie W, Kao H Y, Ordentlich P, Tsai C C, Hon M, Evans R M (2001) Genes Dev. 15(9): 1140-51.
39. Watanabe M, Yanagisawa J, Kitagawa H, Takeyama K, Ogawa S, Arao Y, Suzawa M, Kobayashi Y, Yano T, Yoshikawa H, Masuhiro Y, Kato S. (2001) EMBO J; 20(6): 1341-52.
40. Powers C A, Mathur M, Raaka B M, Ron D, Samuels H H. (1998) Mol Endocrinol. 12(1):4-18.
41. Eggert M, Michel J, Schneider S, Bornfleth H, Baniahmad A, Fackelmayer F O, Schmidt S, Renkawitz R. (1997) J Biol Chem. 272(45): 28471-8.
42. Mathur M, Tucker P W, Samuels H H (2001) Mol Cell Biol. 21(7): 2298-311.
43. Urban R J, Bodenburg Y H, Wood T G (2002) Am J Physiol Endocrinol Metab. 2002 Sep;283(3):E423-7.
44. Pieber D, Allport V C, Hills F, Johnson M, Bennett P R (2001) Mol Hum Reprod. 9:875-9
45. Henderson D, Wilson T (2001) Am J Obstet Gynecol. 2001 Sep;185(3):579-85.

1-13. (canceled)

14. A method for preventing or treating a condition mediated by a steroid receptor in a subject comprising administering a PSF Polypeptide, a PSF Polynucleotide, a PSF Complex, and/or an agonist or antagonist thereof

15. A method of claim 14 wherein the condition is labor.

16. A method of claim 14 wherein the condition is cancer.

17. A method for identifying a substance that modulates a steroid receptor, a PSF Polypeptide, a PSF Complex, a process mediated by a steroid receptor, degradation of a steroid receptor, a steroid receptor signaling transduction pathway, a condition mediated by a steroid receptor, steroid receptor transactivation, and/or inhibits or potentiates the interaction of a steroid receptor and a PSF Polypeptide, comprising assaying for a substance that inhibits or stimulates a PSF Polypeptide, a PSF Polynucleotide, or PSF Complex.

18. A method of claim 17 for evaluating a substance for its ability to regulate the onset of labor comprising the steps of:
(a) reacting a PSF Polypeptide and a progesterone receptor and a test substance, wherein the PSF Polypeptide and receptor bind to form a complex; and
comparing to a control in the absence of the substance to determine if the substance stimulates or inhibits the binding of the PSF Polypeptide to the receptor and thereby regulates the onset of labor.

19. (canceled)

20. A method of conducting a drug discovery business comprising:
(a) providing a method for identifying a substance as claimed in claim 18;
(b) conducting therapeutic profiling of substances identified in step (a), or further analogs thereof, for efficacy and toxicity in animals; and
(c) formulating a pharmaceutical preparation including one or more substances identified in step (b) as having an acceptable therapeutic profile.

21. A method of claim 17 wherein the steroid receptor is a progesterone receptor, a glucocorticoid receptor, or an androgen receptor.

22. A method of claim 17 wherein the PSF polypeptide comprises a sequence of SEQ ID NOs. 1, 2, 3, 4, 5, 6, or 21, or part thereof.

23. A method of claim 22 wherein a part of a PSF Polypeptide consists of a binding domain of the polypeptide that interacts with a steroid receptor, preferably the part is a RRMII domain, a polypeptide consisting of amino acids 1-150, amino acids 290-370, or amino acids 1-662 of SEQ ID NO. 1.

24. A method of claim 17 wherein the steroid receptor is progesterone receptor comprising SEQ ID NOs. 10, 11, 12, 13, 14, or 15, or SEQ ID NO. 10 with amino acids 1 to 164 missing, or parts thereof.

25. A method of claim 24 wherein a part of a progesterone receptor consists of a binding domain of the polypeptide that interacts with a PSF Polypeptide, preferably a DBD domain or a AF3 domain, a polypeptide consisting of amino acids 1-164 of SEQ ID NO. 10, amino acids 456-650 of SEQ ID NO. 10, amino acids 567-587 of SEQ ID NO. 10, or amino acids 556 to 933 of SEQ ID NO. 10.

26. A method of claim 14 for regulating the onset of labor in a subject comprising inhibiting or stimulating a PSF Polypeptide, PSF Polynucleotide, a complex of a PSF Polypeptide and a progesterone receptor (PSF-PR Complex), or interaction between a PSF Polypeptide and a PR Polypeptide.

27-38. (canceled)

39. A method for identifying pre-term labor or the onset of labor in a subject comprising detecting a PSF Polypeptide, PSF Polynucleotide, and/or PSF Complex in a sample from the subject.

40. A method of claim 39 for diagnosing in a subject a condition requiring regulation of the onset of labor comprising detecting a PSF Polypeptide in a sample from the subject.

41. A method of claim 39 for diagnosing increased risk of pre-term labor in a subject comprising detecting a PSF Polypeptide in a sample from the subject.

42. A method of claim 39, which comprises (a) collecting a sample from the subject; (b) measuring the levels of PSF Polypeptide in the sample; and (c) comparing the levels of PSF Polypeptide in the sample to the levels in subjects with normal pregnancies.

43. A method of claim 42 wherein significantly increased levels in the sample compared to levels in samples from subjects who do not suffer from pre-term labor is indicative of an increased risk of pre-term labor.

44. A pharmaceutical composition adapted for administration to a subject for the prevention or treatment of a condition mediated by a steroid receptor comprising an effective amount of a PSF Polypeptide, PSF Complex, and/or PSF Polynucleotide, or an agonist or antagonist thereof, or an agent, compound or substance identified using a method of claim 17, and a pharmaceutically acceptable carrier, diluent or excipient.

45. (canceled)

46. A pharmaceutical composition of claim 44 wherein the steroid receptor is progesterone receptor.

47-52. (canceled)

53. A kit for carrying out a method of claim 17.

54. (canceled)


SEQUENCE LISTING (22 sequences)

SEQ ID NO: 1 - protein, 707 aa, Homo sapiens
  ref <308>: DATABASE ACCESSION NUMBER: P23246
  ref <309>: DATABASE ENTRY DATE: 2004-06-15
  ref <313>: RELEVANT RESIDUES: (1)..(707)
  ref <308>: DATABASE ACCESSION NUMBER: CAA50283
  ref <309>: DATABASE ENTRY DATE: 1993-11-22
  ref <313>: RELEVANT RESIDUES: (1)..(707)
  ref <308>: DATABASE ACCESSION NUMBER: NP_005057
  ref <309>: DATABASE ENTRY DATE: 2004-10-28
  ref <313>: RELEVANT RESIDUES: (1)..(707)
MSRDRFRSRGGGGGGFHRRGGGGGRGGLHDFRSPPPGMGLNQNRGPMGPGPGQSGPKPPIPPPPPHQQQQQPPPQQPPPQQPPPHQPPPHPQPHQQQQPPPPPQDSSKPVVAQGPGPAPGVGSAPPASSSAPPATPPTSGAPPGSGPGPTPTPPPAVTSAPPGAPPPTPPSSGVPTTPPQAGGPPPPPAAVPGPGPGPKQGPGPGGPKGGKMPGGPKPGGGPGLSTPGGHPKPPHRGGGEPRGGRQHHPPYHQQHHQGPPPGGPGGRSEEKISDSEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLLRQDLMRRQEELRRMEELHNQEMQKRKEMQLRQEEERRRREEEMMIRQREMEEQMRRQREESYSRMGYMDPRERDMRMGGGGAMNMGDPYGSGGQKFPPLGGGGGIGYEANPGVPPATMSGSMMGSDMRTERFGQGGAGPVGGQGPRGMGPGTPAGYGRGREEYEGPNKKPRF

SEQ ID NO: 2 - protein, 707 aa, Homo sapiens
  ref <308>: DATABASE ACCESSION NUMBER: AAH51192
  ref <309>: DATABASE ENTRY DATE: 2004-06-30
  ref <313>: RELEVANT RESIDUES: (1)..(707)
MSRDRFRSRGGGGGGFHRRGGGGGRGGLHDFRSPPPGMGLNQNRGPMGPGPGQSGPKPPIPPPPPHQQQQQPPPQQPPPQQPPPHQPPPHPQPHQQQQPPPPPQDSSKPVVAQGPGPAPGVGSTPPASSSAPPATPPTSGAPPGSGPGPTPTPPPAVTSAPPGAPPPTPPSSGVPTTPPQAGGPPPPPAAVPGPGPGPKQGPGPGGPKGGKMPGGPKPGGGPGLSTPGGHPKPPRRGGGEPRGGRQHHPPYHQQHHQGPPPGGPGGRSEEKISDSEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPTRFAQHGTFEYEYSQRWKSLDEMEKQQREQVEKNMKDAKDKLESEMEDAYHEHQANLLRQDLMRRQEELRRMEELHNQEMQKRKEMQLRQEEERRRREEEMMIRQREMEDQMRRQREESYSRMGYMDPRERDMRMGGGGAMNMGDPYGSGGQKFPPLGGGGGIGYEANPGVPPATMSGSMMGSDMRTERFGQGGAGPVGGQGPRGMGPGTPAGYGRGREEYEGPNKKPRF

SEQ ID NO: 3 - protein, 669 aa, Homo sapiens
MSRDRFRSRGGGGGGFHRRGGGGGRGGLHDFRSPPPGMGLNQNRGPMGPGPGQSGPKPPIPPPPPHQQQQQPPPQQPPPQQPPPHQPPPHPQPHQQQQPPPPPQDSSKPVVAQGPGPAPGVGSAPPASSSAPPATPPTSGAPPGSGPGPTPTPPPAVTSAPPGAPPPTPPSSGVPTTPPQAGGPPPPPAAVPGPGPGPKQGPGPGGPKGGKMPGGPKPGGGPGLSTPGGHPKPPHRGGGEPRGGRQHHPPYHQQHHQGPPPGGPGGRSEEKISDSEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLLRQDLMRRQEELRRMEELHNQEMQKRKEMQLRQEEERRRREEEMMIRQREMEEQMRRQREESYSRMGYMDPRERDMRMGGGGAMNMGDPYGSGGQKFPPLGGGGGIGYEANPGVPPATMSGSMMGSDMVRMIDVG

SEQ ID NO: 4 - protein, 634 aa, Homo sapiens
  ref <308>: DATABASE ACCESSION NUMBER: AAH04534
  ref <309>: DATABASE ENTRY DATE: 2003-11-04
  ref <313>: RELEVANT RESIDUES: (1)..(634)
PQQPPPQQPPPHQPPPHPQPHQQQQPPPPPQDSSKPVVAQGPGPAPGVGSAPPASSSAPPATPPTSGAPPGSGPGPTPTPPPAVTSAPPGAPPPTPPSSGVPTTPPQAGGPPPPPAAVPGPGPGPKQGPGPGGPKGGKMPGGPKPGGGPGLSTPGGHPKPPHRGGGEPRGGRQHHPPYHQQHHQGPPPGGPGGRSEEKISDSEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLLRQDLMRRQEELRRMEELHNQEMQKRKEMQLRQEEERRRREEEMMIRQREMEEQMRRQREESYSRMGYMDPRERDMRMGGGGAMNMGDPYGSGGQKFPPLGGGGGIGYEANPGVPPATMSGSMMGSDMRTERFGQGGAGPVGGQGPRGMGPGTPAGYGRGREEYEGPNKKPRF

SEQ ID NO: 5 - protein, 525 aa, Homo sapiens
  ref <308>: DATABASE ACCESSION NUMBER: AAH27708
  ref <309>: DATABASE ENTRY DATE: 2003-09-30
  ref <313>: RELEVANT RESIDUES: (1)..(525)
MSRDRFRSRGGGGGGFHRRGGGGGRGGLHDFRSPPPGMGLNQNRGPMGPGPGQSGPKPPIPPPPPHQQQQQPPPQQPPPQQPPPHQPPPHPQPHQQQQPPPPPQDSSKPVVAQGPGPAPGVGSAPPASSSAPPATPPTSGAPPGSGPGPTPTPPPAVTSAPPGAPPPTPPSSGVPTTPPQAGGPPPPPAAVPGPGPGPKQGPGPGGPKGGKMPGGPKPGGGPGLSTPGGHPKPPHRGGGEPRGGRQHHPPYHQQHHQGPPPGGPGGRSEEKISDSEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLKKKKKK

SEQ ID NO: 6 - protein, 396 aa, Homo sapiens
  ref <308>: DATABASE ACCESSION NUMBER: CAA34747
  ref <309>: DATABASE ENTRY DATE: 1995-07-19
  ref <313>: RELEVANT RESIDUES: (1)..(396)
EFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLLRQDLMRRQEELRRMEELHNQEMQKRKEMQLRQEEERRRREEEMMIRQREMEEQMRRQREESYSRMGYMDPRERDMRMGGGGAMNMGDPYGSGGQKFPPLGGGGGIGYEANPGVPPATMSGSMMGSDMRTERFGQGGAGPVGGQGPRGMGPGTPAGYGRGREEYEGPNKKPRF

SEQ ID NO: 7 - DNA, 3071 bp, Homo sapiens
  ref <308>: DATABASE ACCESSION NUMBER: NM_005066
  ref <309>: DATABASE ENTRY DATE: 2004-10-28
  ref <313>: RELEVANT RESIDUES: (1)..(3071)
  ref <308>: DATABASE ACCESSION NUMBER: S56626
  ref <309>: DATABASE ENTRY DATE: 1993-07-26
  ref <313>: RELEVANT RESIDUES: (1)..(3071)
  ref <308>: DATABASE ACCESSION NUMBER: X70944
  ref <309>: DATABASE ENTRY DATE: 1993-11-22
  ref <313>: RELEVANT RESIDUES: (1)..(3071)
ccgccattttgtgagaagcaaggtggcctccacgtttcctgagcgtcttcttcgcttttgcctcgaccgccccttgaccacagacatgtctcgggatcggttccggagtcgtggcggtggcggtggtggcttccacaggcgtggaggaggcggcggccgcggcggcctccacgacttccgttctccgccgcccggcatgggcctcaatcagaatcgcggccccatgggtcctggcccgggccagagcggccctaagcctccgatcccgccaccgcctccacaccaacagcagcaacagccaccaccgcagcagccaccgccgcagcagccgccaccgcatcagccgccgccgcatccacagccgcatcagcagcagcagccgccgccaccgccgcaggactcttccaagcccgtcgttgctcagggacccggccccgctcccggagtaggcagcgcaccaccagcctccagctcggccccgcccgccactccaccaacctcgggggccccgccagggtccgggccaggcccgactccgaccccgccgcctgcagtcacctcggcccctcccggggcgccgccacccaccccgccaagcagcggggtccctaccacacctcctcaggccggaggcccgccgcctccgcccgcggcagtcccgggcccgggtccagggcctaagcagggcccaggtccgggtggtcccaaaggcggcaaaatgcctggcgggccgaagccaggtggcggcccgggcctaagtacgcctggcggccaccccaagccgccgcatcgaggcggcggggagccccgcgggggccgccagcaccacccgccctaccaccagcagcatcaccaggggcccccgcccggcgggcccggcggccgcagcgaggagaagatctcggactcggaggggtttaaagccaatttgtctctcttgaggaggcctggagagaaaacttacacacagcgatgtcggttgtttgttgggaatctacctgctgatatcacggaggatgaattcaaaagactatttgctaaatatggagaaccaggagaagtttttatcaacaaaggcaaaggattcggatttattaagcttgaatctagagctttggctgaaattgccaaagccgaactggatgatacacccatgagaggtagacagcttcgagttcgctttgccacacatgctgctgccctttctgttcgtaatctttcaccttatgtttccaatgaactgttggaagaagcctttagccaatttggtcctattgaaagggctgttgtaatagtggatgatcgtggaagatctacagggaaaggcattgttgaatttgcttctaagccagcagcaagaaaggcatttgaacgatgcagtgaaggtgttttcttactgacgacaactcctcgtccagtcattgtggaaccacttgaacaactagatgatgaagatggtcttcctgaaaaacttgcccagaagaatccaatgtatcaaaaggagagagaaacccctcctcgttttgcccagcatggcacgtttgagtacgaatattctcagcgatggaagtctttggatgaaatggaaaaacagcaaagggaacaagttgaaaaaaacatgaaagatgcaaaagacaaattggaaagtgaaatggaagatgcctatcatgaacatcaggcaaatcttttgcgccaagatctgatgagacgacaggaagaattaagacgcatggaagaacttcacaatcaagaaatgcagaaacgtaaagaaatgcaattgaggcaagaggaggaacgacgtagaagagaggaagagatgatgattcgtcaacgtgagatggaagaacaaatgaggcgccaaagagaggaaagttacagccgaatgggctacatggatccacgggaaagagacatgcgaatgggtggcggaggagcaatgaacatgggagatccctatggttcaggaggccagaaatttccacctctaggaggtggtggtggcataggttatgaagctaatcctggcgttccaccagcaaccatgagtggttccatgatgggaagtgacatgcgtactgagcgctttgggcagggaggtgcggggcctgtgggtggacagggtcctagaggaatggggcctggaactccagcaggatatggtagagggagagaagagtacgaaggcccaaacaaaaaaccccgattttagatgtgatatttaggctttcattccagtttgttttgtttttttgtttagataccaatcttttaaattcttgcattttagtaagaaagctatctttttatggatgttagcagtttattgacctaatatttgtaaatggtctgtttgggcaggtaaaattatgtaatgcagtgtttggaacaggagaatttttttttcctttttatttctttattttttcttttttactgtataatgtccctcaagtttatggcagtgtaccttgtgccactgaatttccaaagtgtaccaatttttttttttttactgtgcttcaaataaatagaaaaatagttataatattggatcttcaactttgccattcatgcttctatgcatattaggctacgtattccacattgaaagcatgagagtgtctaggcctttgaatggcatatgccatttctgggaaatgcatctggaggctaagtattgctttctacaaataattgccccctttgttttaaaaagaagaaatgcatattgaagtagtttgatgatttgtttggcatataggaagcacgctggtgctaagtattttttaaatggttatgtaagcaaagctgaactgtaaatcttcaggaatatgtattaagattgtggaatgggtgtaagacaattggtagggggtgaaagtgggtttgattaaatggatcttttatggccctatgatctatcctttacttgaaagcttttgaaaagtggaaaggtcattttgttgcatttccccatttcttgtttttaaaagaccaacaaatctcaagccctataaatggcttgtattgaacttttacatttgaattaaagatgttaaacatgaaaaaaa

SEQ ID NO: 8 - DNA, 2622 bp, Homo sapiens
  ref <308>: DATABASE ACCESSION NUMBER: BC051192
  ref <309>: DATABASE ENTRY DATE: 2004-06-30
  ref <313>: RELEVANT RESIDUES: (1)..(2622)
tctgtgtcatccgccattttgtgagaagcaaggtggcctccacgtttcctgagcgtcttcttcgcttttgcctcgaccgccccttgaccacagacatgtctcgggatcggttccggagtcgtggcggtggcggtggtggcttccacaggcgtggaggaggcggcggccgcggcggcctccacgacttccgttctccgccgcccggcatgggcctcaatcagaatcgcggccccatgggtcctggcccgggccagagcggccctaagcctccgatcccgccaccgcctccacaccaacagcagcaacagccaccaccgcagcagccaccgccgcagcagccgccaccgcatcagccgccgccgcatccacagccgcatcagcagcagcagccgccgccaccgccgcaggactcttccaagcccgtcgttgctcagggacccggccccgctcccggagtaggcagcacaccaccagcctccagctcggccccgcccgccactccaccaacctcgggggccccgccagggtccgggccaggcccgactccgaccccgccgcctgcagtcacctcggcccctcccggggcgccgccacccaccccgccaagcagcggggtccctaccacacctcctcaggccggaggcccgccgcctccgcccgcggcagtcccgggcccgggtccagggcctaagcagggcccaggtccgggtggtcccaaaggcggcaaaatgcctggcgggccgaagccaggtggcggcccgggcctaagtacgcctggcggccaccccaagccgccgcgtcgaggcggcggggagccccgcgggggccgccagcaccacccgccctaccaccagcagcatcaccaggggcccccgcccggcgggcccggcggccgcagcgaggagaagatctcggactcggaggggtttaaagccaatttgtctctcttgaggaggcctggagagaaaacttacacacagcgatgtcggttgtttgttgggaatctacctgctgatatcacggaggatgaattcaaaagactatttgctaaatatggagaaccaggagaagtttttatcaacaaaggcaaaggattcggatttattaagcttgaatctagagctttggctgaaattgccaaagccgaactggatgatacacccatgagaggtagacagcttcgagttcgctttgccacacatgctgctgccctttctgttcgtaatctttcaccttatgtttccaatgaactgttggaagaagcctttagccaatttggtcctattgaaagggctgttgtaatagtggatgatcgtggaagatctacagggaaaggcattgttgaatttgcttctaagccagcagcaagaaaggcatttgaacgatgcagtgaaggtgttttcttactgacgacaactcctcgtccagtcattgtggaaccacttgaacaactagatgatgaagatggtcttcctgaaaaacttgcccagaagaatccaatgtatcaaaaggagagagaaacccctactcgttttgcccagcatggcacgtttgagtacgaatattctcagcgatggaagtctttggatgaaatggaaaaacagcaaagggaacaagttgaaaaaaacatgaaagatgcaaaagacaaattggaaagtgaaatggaagatgcctatcatgaacatcaggcaaatcttttgcgccaagatctgatgagacgacaggaagaattaagacgcatggaagaacttcacaatcaagaaatgcagaaacgtaaagaaatgcaattgaggcaagaggaggaacgacgtagaagagaggaagagatgatgattcgtcaacgtgagatggaagaccaaatgaggcgccaaagagaggaaagttacagccgaatgggctacatggatccacgggaaagagacatgcgaatgggtggcggaggagcaatgaacatgggagatccctatggttcaggaggccagaaatttccacctctaggaggtggtggtggcataggttatgaagctaatcctggcgttccaccagcaaccatgagtggttccatgatgggaagtgacatgcgtactgagcgctttgggcagggaggtgcggggcctgtgggtggacagggtcctagaggaatggggcctggaactccagcaggatatggtagagggagagaagagtacgaaggcccaaacaaaaaaccccgattttagatgtgatatttaggctttcattccagtttgttttgtttttttgtttagataccaatcttttaaattcttgcattttagtaagaaagctatctttttatggatgttagcagtttattgacctaatatttgtaaatggtctgtttgggcaggtaaaattatgtaatgcagtgtttggaacaggagaatttttttttcctttttatttctttattttttcttttttactgtataatgtccctcaagtttatggcagtgtaccttgtgccactgaatttccaaagtgtaccaatttttttttttttactgtgcttcaaataaatagaaaaatagttataaaaaaaaaaaaaaaaaaaaaaaaaaaaaaaaaaaaaaaaaaaaaaaaaaaaaaaaaaaaaaaaaaaaa

SEQ ID NO: 9 - DNA, 2021 bp, Homo sapiens
  ref <308>: DATABASE ACCESSION NUMBER: X16850
  ref <309>: DATABASE ENTRY DATE: 1995-07-19
  ref <313>: RELEVANT RESIDUES: (1)..(2021)
gaattcaaaagactatttgctaaatatggagaaccaggagaagtttttatcaacaaaggcaaaggattcggatttattaagcttgaatctagagctttggctgaaattgccaaagccgaactggatgatacacccatgagaggtagacagcttcgagttcgctttgccacacatgctgctgccctttctgttcgtaatctttcaccttatgtttccaatgaactgttggaagaagcctttagccaatttggtcctattgaaagggctgttgtaatagtggatgatcgtggaagatctacagggaaaggcattgttgaatttgcttctaagccagcagcaagaaaggcatttgaacgatgcagtgaaggtgttttcttactgacgacaactcctcgtccagtcattgtggaaccacttgaacaactagatgatgaagatggtcttcctgaaaaacttgcccagaagaatccaatgtatcaaaaggagagagaaacccctcctcgttttgcccagcatggcacgtttgagtacgaatattctcagcgatggaagtctttggatgaaatggaaaaacagcaaagggaacaagttgaaaaaaacatgaaagatgcaaaagacaaattggaaagtgaaatggaagatgcctatcatgaacatcaggcaaatcttttgcgccaagatctgatgagacgacaggaagaattaagacgcatggaagaacttcacaatcaagaaatgcagaaacgtaaagaaatgcaattgaggcaagaggaggaacgacgtagaagagaggaagagatgatgattcgtcaacgtgagatggaagaacaaatgaggcgccaaagagaggaaagttacagccgaatgggctacatggatccacgggaaagagacatgcgaatgggtggcggaggagcaatgaacatgggagatccctatggttcaggaggccagaaatttccacctctaggaggtggtggtggcataggttatgaagctaatcctggcgttccaccagcaaccatgagtggttccatgatgggaagtgacatgcgtactgagcgctttgggcagggaggtgcggggcctgtgggtggacagggtcctagaggaatggggcctggaactccagcaggatatggtagagggagagaagagtacgaaggcccaaacaaaaaaccccgattttagatgtgatatttaggctttcattccagtttgttttgtttttttgtttagataccaatcttttaaattcttgcattttagtaagaaagctatctttttatggatgttagcagtttattgacctaatatttgtaaatggtctgtttgggcaggtaaaattatgtaatgcagtgtttggaacaggagaattttttttcctttttatttctttattttttcttttttactgtataatgtccctcaagtttatggcagtgtaccttgtgccactgaatttccaaagtgtaccaatttttttttttttactgtgcttcaaataaatagaaaaatagttataatattgatcttcaactttgccattcatgcttctatgcatattaggctacgtattccacattgaaagcatgagagtgtctaggcctttgaatggcatatgccatttctgggaaatgcatctggaggctaagtattgctttctacaaataattgccccctttgttttaaaaagaagaaatgcatattgaagtagtttgatgatttgtttggcatataggaagcacgctggtgctaagtattttttaaatggttatgtaagcaaagctgaactgtaaatcttcaggaatatgtattaagattgtggaatgggtgtaagacaattggtagggggtgaaagtgggtttgattaaatggatcttttatggccctatgatctatcctttacttgaaagcttttgaaaagtggaaaggtcattttgttgcatttccccatttcttgtttttaaaagaccaacaaatctcaagccctataaatggcttgtattgaacccgaattc

SEQ ID NO: 10 - protein, 933 aa, Homo sapiens
  ref <308>: DATABASE ACCESSION NUMBER: NP_000917
  ref <309>: DATABASE ENTRY DATE: 2004-10-27
  ref <313>: RELEVANT RESIDUES: (1)..(933)
  ref <308>: DATABASE ACCESSION NUMBER: AAS00096
  ref <309>: DATABASE ENTRY DATE: 2004-01-31
  ref <313>: RELEVANT RESIDUES: (1)..(933)
  ref <308>: DATABASE ACCESSION NUMBER: AAD01587
  ref <309>: DATABASE ENTRY DATE: 1999-01-05
  ref <313>: RELEVANT RESIDUES: (1)..(933)
  ref <308>: DATABASE ACCESSION NUMBER: AAA60081
  ref <309>: DATABASE ENTRY DATE: 1995-01-07
  ref <313>: RELEVANT RESIDUES: (1)..(933)
  ref <308>: DATABASE ACCESSION NUMBER: P06401
  ref <309>: DATABASE ENTRY DATE: 2005-01-25
  ref <313>: RELEVANT RESIDUES: (1)..(933)
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDSSESEESAGPLLKGKPRALGGAAAGGGAAACPPGAAAGGVALVPKEDSRFSAPRVALVEQDAPMAPGRSPLATTVMDFIHVPILPLNHALLAARTRQLLEDESYDGGAGAASAFAPPRTSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPLGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 11 - protein, 831 aa, Homo sapiens
  ref <308>: DATABASE ACCESSION NUMBER: BAB91074
  ref <309>: DATABASE ENTRY DATE: 2002-04-24
  ref <313>: RELEVANT RESIDUES: (1)..(831)
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 12 - protein, 695 aa, Homo sapiens
  ref <308>: DATABASE ACCESSION NUMBER: BAC06585
  ref <309>: DATABASE ENTRY DATE: 2004-09-03
  ref <313>: RELEVANT RESIDUES: (1)..(695)
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGFRNLHIDDQITLIQYSWMSLMVFGLGWRSYKHVSGQMLYFAPDLILNDSFGRATKSNPV

SEQ ID NO: 13 - protein, 764 aa, Homo sapiens
  ref <308>: DATABASE ACCESSION NUMBER: BAC11011
  ref <309>: DATABASE ENTRY DATE: 2004-09-03
  ref <313>: RELEVANT RESIDUES: (1)..(764)
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNESHRSLSSFKLAKKSSSV

SEQ ID NO: 14 - protein, 690 aa, Homo sapiens [sequence omitted]

SEQ ID NO: 15 - protein, 803 aa, Homo sapiens
  ref <308>: DATABASE ACCESSION NUMBER: BAC11013
  ref <309>: DATABASE ENTRY DATE: 2004-09-03
  ref <313>: RELEVANT RESIDUES: (1)..(803)
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNESHRSLSSFKLAKKSSSV

SEQ ID NO: 16 - protein, 14 aa, Homo sapiens
FGQGGAGPVGGQGP

SEQ ID NO: 17 - DNA, 7 bp, Homo sapiens
ctgagtc

SEQ ID NO: 18 - protein, 13 aa, Homo sapiens
YGEPGEVFINKGK

SEQ ID NO: 19 - protein, 12 aa, Homo sapiens
GIVEFASKPAAR

SEQ ID NO: 20 - protein, 14 aa, Homo sapiens
FAQHGTEEYEYSQR

SEQ ID NO: 21 - protein, 699 aa, Mus musculus
  ref <308>: DATABASE ACCESSION NUMBER: NP_076092
  ref <309>: DATABASE ENTRY DATE: 2004-08-24
  ref <313>: RELEVANT RESIDUES: (1)..(699)
MSRDRFRSRGGGGGGFHRRGGGGGRGGLHDFRSPPPGMGLNQNRGPMGPGPGGPKPPLPPPPPHQQQQQPPPQQPPPQQPPPHQQPPPHQPPHQQPPPPPQESKPVVPQGPGSAPGVSSAPPPAVSAPPANPPTTGAPPGPGPTPTPPPAVPSTAPGPPPPSTPSSGVSTTPPQTGGPPPPPAGGAGPGPKPGPGPGGPKGGKMPGGPKPGGGPGMGAPGGHPKPPHRGGGEPRGGRQHHAPYHQQHHQGPPPGGPGPRTEEKISDSEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLLRQDLMRRQEELRRMEELHSQEMQKRKEMQLRQEEERRRREEEMMIRQREMEEQMRRQREESYSRMGYMDPRERDMRMGGGGTMNMGDPYGSGGQKFPPLGGGGGIGYEANPGVPPATMSGSMMGSDMRTERFGQGGAGPVGGQGPRGMGPGTPAGYGRGREEYEGPNKKPRF

SEQ ID NO: 22 - protein, 7 aa, Homo sapiens
VRMIDVG